(19) United States
(12) Patent Application Publication
(10) Pub. No.: US 2025/0382351 A1
(43) Pub. Date: Dec. 18, 2025

(54) COMPOSITIONS CONTAINING ACTIVATABLE ANTIBODIES

(71) Applicant: CYTOMX THERAPEUTICS, INC., SOUTH SAN FRANCISCO, CA (US)

(72) Inventors: Sarah PATRICK, Menlo Park, CA (US); Claus KREBBER, Palo Alto, CA (US); Sridhar VISWANATHAN, Menlo Park, CA (US); Shanti Gonela DUVUR, Fremont, CA (US); Eric URENO, Daly City, CA (US)

(73) Assignee: CYTOMX THERAPEUTICS, INC., SOUTH SAN FRANCISCO, CA (US)

(21) Appl. No.: 19/316,660

(22) Filed: Sep. 2, 2025

Related U.S. Application Data
(62) Division of application No. 17/227,029, filed on Apr. 9, 2021, now Pat. No. 12,428,470.
(60) Provisional application No. 63/007,776, filed on Apr. 9, 2020.

(51) Int. Cl. C07K 16/00; C07K 1/22; C07K 16/28

(52) U.S. Cl. CPC C07K 16/00; C07K 1/22; C07K 16/2803; C07K 16/2818; C07K 16/2827

(57) ABSTRACT

The invention provides a composition including an intact activatable antibody and a clipped variant thereof, methods of separating clipped variants of intact activatable antibodies from intact activatable antibodies and related methods including methods for determining or monitoring a relative percentage of an activatable antibody and a clipped variant thereof during a composition production process.

### CROSS REFERENCE TO RELATED APPLICATIONS

This application is a divisional of U.S. application Ser. No. 17/227,029 filed Apr. 9, 2021, which claims priority benefit of U.S. provisional application No. 63/007,776, filed Apr. 9, 2020, the entire contents of which are incorporated herein by reference.

### REFERENCE TO SEQUENCE LISTING

The present application includes a Sequence Listing filed in electronic format. The Sequence Listing is entitled “4862-110.xml” created on Aug. 27, 2025, and is 393,720 bytes in size. The information in the electronic format of the Sequence Listing is part of the present application and is incorporated herein by reference in its entirety.

### FIELD OF THE INVENTION

The present disclosure relates to compositions and methods relating to intact activatable antibodies and clipped variants thereof, including compositions and methods for making, purifying, measuring, monitoring, and using the compositions.

### BACKGROUND

Monoclonal antibodies are a growing class of therapeutic compounds each designed to bind to a target antigen implicated in any one of a number of clinical indications. To date, there is an expansive list of monoclonal antibody products, either at an investigational stage or approved as a new drug. While the burgeoning growth of this product class has resulted in significant advances in the processes used to manufacture them, product aggregation remains a significant issue that must be addressed during the development of a manufacturing process for each new monoclonal antibody drug. Varsha, et al. BioPharm International vol. 26, Issue 3. Product aggregation is undesirable as it results in lower yields of drug product, and potential safety issues if not removed from the drug product composition. Id. For example, aggregation may cause the formation of subvisible particles that may expose normally unexposed epitopes, leading to potentially increased immunogenicity if administered to a patient. Id.

The aggregates are typically large, tangled clusters of denatured antibody molecules that form irreversibly either during product expression in the cell culture, during product purification in downstream processing, or during storage. Id. A variety of factors may contribute to the formation of aggregates. Improvements in the safety and/or efficacy of antibody-based therapeutics have been sought, and in certain cases, achieved, by modifying the structure of the molecule. However, in certain instances, these structural changes introduce additional challenges with regards to the manufacture of purified antibody-based therapeutics.

Accordingly, new processes for manufacturing antibody-based therapeutic compounds would be highly desirable.

### SUMMARY OF THE INVENTION

In one aspect, the present disclosure includes a composition including an intact activatable antibody and a clipped variant thereof, wherein the clipped variant is present in a reduced amount. In some aspects, the clipped variant thereof includes an antigen binding domain (AB) and at least a portion of a cleavable moiety (CM). In some aspects, the clipped variant lacks a masking moiety (MM) from at least one prodomain.

In some aspects, the composition comprises at least about 90% intact activatable antibody, as measured by reducing SDS-cGE, less than about 10% clipped variant, as determined by reducing SDS-cGE, less than about 5% high molecular weight species (HMWS), as determined by SE-HPLC, and less than about 150 ppm host cell proteins (HCP), as determined by a corresponding HCP ELISA. In some aspects, the composition includes greater than 95% intact activatable antibody and 0.05 to 5% clipped variant. In some aspects, the composition includes greater than 90% intact activatable antibody, 0.05 to 5% clipped variant, less than 150 ppm host cell proteins (HCP), and/or less than 5% HMWS. In some aspects, the composition includes greater than 96% intact activatable antibody, 0.05 to 4% clipped variant, less than 150 ppm HCP and less than 5% HMWS. In some aspects, the composition includes greater than 97% intact activatable antibody, 0.05 to 3% clipped variant, less than 150 ppm host cell proteins (HCP) and less than 5% HMWS. In some aspects, the composition includes greater than 98% intact activatable antibody, 0.05 to 2% clipped variant, less than 150 ppm host cell proteins (HCP) and less than 5% HMWS. In some aspects, the composition includes greater than 99% intact activatable antibody, 0.05 to 1% clipped variant, less than 150 ppm host cell proteins (HCP) and less than 5% HMWS. In some aspects, the present disclosure includes a container, vial, syringe, or injector device containing the composition.

In one aspect, the present disclosure includes a process for producing a composition including: (A) greater than 95% intact activatable antibody comprising a MM, a CM, and a AB; and (B) 0.05 to 5% clipped variant thereof, the process including loading an aqueous feedstock comprising water, (A), (B), and a first salt onto a chromatography column, wherein the chromatography column comprises a stationary phase that comprises a support matrix and hydrophobic ligands bound thereto, and eluting the chromatography column with an eluent comprising water and a second salt to obtain the composition. In one aspect, the process includes reducing the amount of clipped variant in the process stream by 75 to 90%. In one aspect, the process includes reducing the amount of HCP in the process stream by 75 to 90%. In one aspect, the process includes reducing the amount of HMWS in the process stream by 75 to 90%. In one aspect, the process includes reducing the amount of clipped variant, HCP, and HMWS in the process stream by 70 to 95%.

In one aspect, the present disclosure includes a method of separating an intact activatable antibody from a clipped variant thereof that has an amino acid sequence that is at least 90%, 91%, 92%, 93%, 94%, 95%, 96%, 97%, 98%, or 99% identical to the amino acid sequence of the intact activatable antibody, including (i) loading an aqueous feedstock comprising water, the intact activatable antibody, the clipped variant thereof, and a first salt onto a chromatography column, wherein the chromatography column comprises a stationary phase that comprises a support matrix and hydrophobic ligands bound thereto, and (ii) eluting the chromatography column with an eluent comprising water and a second salt to obtain a composition wherein the amount of the clipped variant in the process stream is reduced by at least 70%. In some embodiments, the amount of the clipped variant in the process stream is reduced by at least 75%, 80%, 85%, or 90%.

In one aspect, the present disclosure includes a composition and process for making a composition having at least 90%, 91%, 92%, 93%, 94%, 95%, 96%, 97%, 98%, or 99% of the total protein by weight in the composition in the form of intact activatable antibody, and 0.1 to 10% of the total protein by weight in the form of aggregated and clipped variants thereof.

In one aspect, the present disclosure includes a method for producing a pharmaceutical composition including an intact activatable antibody and less than 5% clipped variant thereof, less than 5% aggregates thereof, less than 150 ppm HCP, or a combination thereof, the method including loading an aqueous feedstock comprising water, (A), (B), and a first salt onto a chromatography column, wherein the chromatography column comprises a stationary phase that comprises a support matrix and hydrophobic ligands bound thereto, and eluting the chromatography column with an eluent comprising water and a second salt to obtain the composition.

In some aspects, the present disclosure includes administering the composition disclosed herein to a subject in need thereof, e.g., a patient suffering from a cancer, an inflammatory disease, an autoimmune disease, or a combination thereof. In some aspects, the present disclosure includes administering a composition disclosed herein with a subtoxic dose of a clipped variant of an activatable antibody and a dose of an activatable antibody that is activated in a disease microenvironment. In some aspects, the present disclosure includes expanding a therapeutic window of treating a subject by administering a composition of the present disclosure.

In one aspect, the present invention provides a process for producing a purified composition of intact activatable antibody, the process comprising:
(a) loading an aqueous feedstock comprising water, an intact activatable antibody, a clipped impurity, and a first salt onto a chromatography column,
wherein the chromatography column comprises a stationary phase that comprises a support matrix and ligands bound thereto,
   wherein the ligands comprise a hydrophobic substituent, and
wherein the intact activatable antibody comprises (i) at least a first antigen binding domain (AB) that has a specific binding affinity for a first biological target, and (ii) a first prodomain,
   wherein the at least first AB comprises a first antibody light variable domain (VL) and a first antibody heavy variable domain (VH),
   wherein the first prodomain comprises a first masking moiety (MM) and a first cleavable moiety (CM), and
   wherein the first AB is coupled to the first prodomain; and
(b) eluting the chromatography column with an eluent comprising water and a second salt to generate an eluate that comprises a purified composition comprising intact activatable antibody,
wherein the eluate is substantially depleted of the clipped impurity.

In another aspect, the aqueous feedstock further comprises other impurities, such as, for example, host cell proteins (HCP) and/or high molecular weight species (HMWS), wherein quantities of these impurities are substantially reduced in the eluate.

In a further aspect, the present invention provides purified intact activatable antibody compositions having either no or very low residual quantities of clipped impurity.

In a still further aspect, the present invention provides purified intact activatable antibody compositions having either no or very low residual quantities of clipped impurity, host cell protein (HCP), and high molecular weight species (HMWS).

In another aspect, the present disclosure includes a method for determining or monitoring a relative quantity of an activatable antibody and a clipped variant thereof during a composition production process, by subjecting a sample composition comprising a population of activatable antibody and a population of clipped variants thereof to a gel capillary electrophoresis procedure; separating the population of activatable antibody from the population of clipped variants thereof in the gel capillary electrophoresis procedure; and quantifying the relative amounts of the population of activatable antibody and the population of clipped variants thereof by determining the peak area corresponding to intact prodomain-encoding polypeptide and the peak area corresponding to clipped prodomain-encoding polypeptide(s) thereof.

### BRIEF DESCRIPTION OF THE DRAWINGS

FIG. 1A depicts the results of a mass spectrophotometry analysis of a sample obtained from a bioharvest of activatable anti-PDL1 antibody. The results show a single peak corresponding to the expected molecular weight for the corresponding single-arm clipped impurity. No peaks were observed at the expected molecular weights for fully clipped impurity.

FIG. 1B depicts the results of a mass spectrophotometry analysis of a sample obtained from a bioharvest of activatable anti-PD1 antibody. The results show a single peak corresponding to the expected molecular weight for the corresponding single-arm clipped impurity. No peaks were observed at the expected molecular weights for fully clipped impurity.

FIG. 1C depicts the results of a mass spectrophotometry analysis of a sample obtained from a bioharvest of activatable anti-CD166 antibody. The results show a single peak corresponding to the expected molecular weight for the corresponding single-arm clipped impurity. No peaks were observed at the expected molecular weights for fully clipped impurity.

FIG. 2A schematically depicts the structures of an intact activatable antibody and clipped variants thereof.

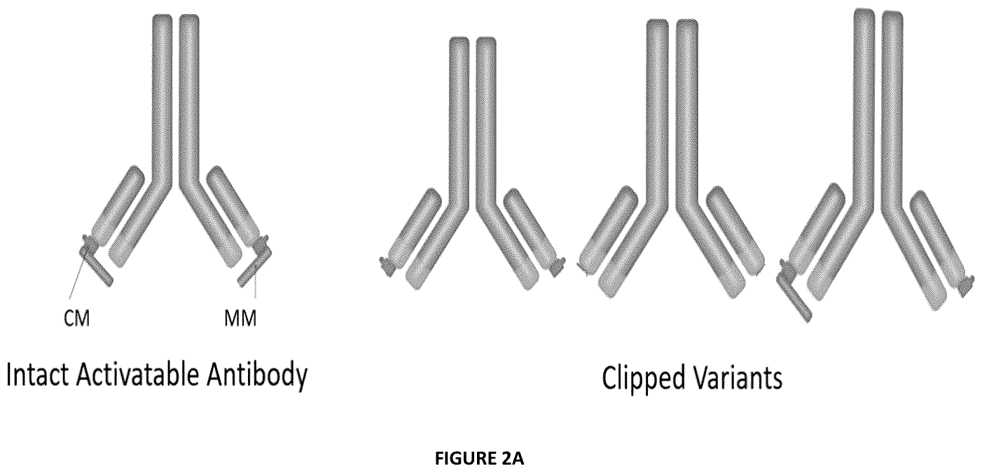

FIG. 2B schematically depicts a mixture of intact activatable antibody and clipped variants thereof as may be present in a composition prior to treatment with the method of the present disclosure.

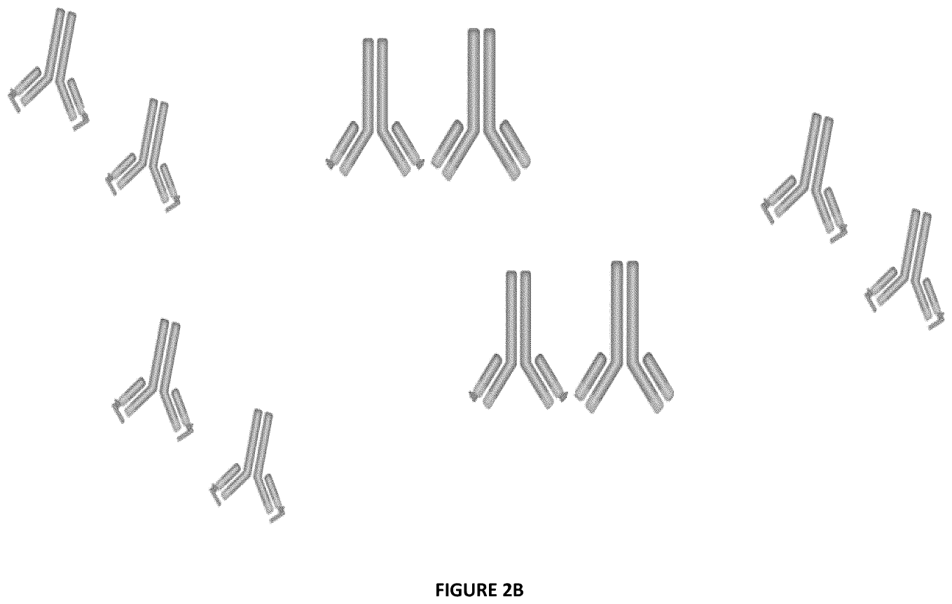

FIG. 3A schematically depicts: (1) administration of the mixture of FIG. 2B to cells in a subject; (2) binding of intact activatable antibody and clipped variants thereof to the subject's cells after administration; (3) binding of clipped variants from the composition to healthy tissues; and (4) activation of intact activatable antibody in the environment of diseased (e.g., tumor) tissues and binding of the activated antibodies thereto.

FIG. 3B schematically depicts: (1) binding of intact activatable antibody present in a composition after treatment with a method of the present disclosure upon administration to a subject; (2) lack of binding to healthy tissues of the administered intact activatable antibodies; and (3) activation of intact activatable antibody in the environment of only diseased (e.g., tumor) tissues and binding of the activated antibodies thereto.

FIG. 4A depicts the results of reducing sodium dodecyl sulfate (SDS) capillary gel electrophoresis (SDS-cGE) performed on a sample comprising an anti-CD-166 activatable antibody following purification by protein A chromatography.

FIG. 4B depicts the results of reducing SDS-cGE performed on a sample of the eluate following anion ion exchange chromatography (anion IEX) for a sample comprising an anti-CD-166 activatable antibody. The aqueous feedstock for the anion IEX step comprised the eluate of the protein A chromatography step, the analysis of which is depicted in FIG. 4A.

FIG. 4C depicts the results of reducing SDS capillary gel electrophoresis (SDS-cGE) performed on a sample comprising an anti-CD-166 activatable antibody following purification by hydrophobic interaction chromatography (HIC). The aqueous feedstock for the HIC step comprised the eluate of the anion IEX step, the analysis of which is depicted in FIG. 4B.

FIG. 5 depicts the evaluation of cation exchange chromatography for purifying an aqueous feedstock comprising intact activatable anti-CD166 antibody and a corresponding clipped impurity as described in Example 2. The column was eluted with a NaCl gradient by mixing a first buffer (25 mM NaOAc, 500 mM NaCl, pH 5.0 (80 CV grad)) into a second buffer (25 mM NaOAc, 25 mM NaCl, pH 5.0). Line “A” represents absorbance at 280 nm, Line “B” represents absorbance at 254 nm, Line “C” represents the conductivity in mS/cm, and Line “D” represents the percent of the first buffer mixed into the second buffer. The left y-axis presents absorbance in mAU, the right y-axis presents conductivity in mS/cm, and the x-axis presents fractions in mL.

### DETAILED DESCRIPTION OF THE INVENTION

Toxicities due to broad target expression have limited the therapeutic effectiveness of monoclonal antibody therapies. To address this, recombinantly produced activatable antibodies that include an antigen binding domain (AB), a cleavable moiety (CM), and a masking moiety (MM) that is capable of inhibiting the specific binding of the AB to its target have been produced. Such activatable antibodies behave like an antibody with respect to binding specificity to a biological target only after activation by exposure to certain proteases, particularly proteases that are upregulated in a localized disease environment (e.g., a tumor microenvironment).

The present inventors have now found that, due to proteases present in host cells during the manufacturing process and the relative lability of protease substrates, compositions containing activatable antibodies are often found to contain a significant proportion of clipped variants that lack the MM and therefore are free to indiscriminately bind to targets on healthy cells, thereby potentially causing the dose-dependent toxicities and side effects associated with monoclonal antibodies. The presence of clipped variants in a therapeutic dose may have the effect of reducing the dose of intact activatable antibody in systemic circulation that is available to reach the target tissues (e.g., cancerous tissue) due to sequestration of the clipped variants in normal tissue. In addition to the presence of clipped variants, the present inventors have also observed that certain compositions containing activatable antibodies also contain significant portions of high molecular weight species due to aggregation, which may reduce efficacy and increase immunogenicity. Particularly, the present inventors have now found that clipped variants are similar in size and identical in amino acid sequence to the activatable antibody parent except for the truncation caused by cleavage. Because of the similarities in size and structure and physicochemical properties of the clipped variant and the intact activatable antibody, it is challenging to separate the clipped variants from the intact activatable antibody parent to obtain a composition having high purity (e.g., a high percentage of intact, non-aggregated activatable antibody), in high yield. As described below, the present inventors have discovered methods for purifying activatable antibody compositions to selectively remove clipped variants, resulting in compositions comprising high levels of intact activatable antibody with low or no detectable levels of clipped variants.

### Definitions

The terms “activatable antibody”, “protease-activatable antibody”, and “intact activatable antibody” are used interchangeably herein to refer to a recombinantly produced “masked” binding compound that is designed to behave like an antibody with respect to binding specificity to a biological target only after its activation by exposure to certain proteases. Structurally, activatable antibodies comprise: (1) an antigen binding domain (AB) that, when not masked, specifically binds a biological target; and (2) a prodomain coupled (via peptide bonding) to the AB that comprises or consists of a masking moiety (MM) and a cleavable moiety (CM).

The term “amino acid anion” refers herein to an anionic form of an amino acid. The anionic moiety may, for example, be a deprotonated alpha-carboxyl or R-group carboxyl (e.g., the R-group carboxyl moiety in aspartic acid, glutamic acid, and the like), and the like.

The term “amino acid cation” refers herein to a cationic form of an amino acid. The cationic moiety may, for example, be a protonated alpha-amine or R-group amine (e.g., the R group amine moiety in arginine (i.e., the guanidino moiety), tryptophan, asparagine, glutamine, lysine, histidine, and the like), and the like.

The term “amino acid salt” refers herein to a salt of an amino acid. Illustrative amino acid salts include, for example, an arginine hydrochloride, a lysine hydrochloride, and the like.

The terms “antigen binding domain” and “AB” are used interchangeably herein to refer to a binding domain having a specific binding affinity for a biological target, that is formed from one or more polypeptides encoding an antibody light variable domain (VL) and an antibody heavy variable domain (VH).

As used herein, the term “biological target” refers to a protein that is native to a mammalian species.

As used herein, the term “prodomain” refers herein to a peptide having an amino acid sequence that encodes at a minimum, a masking moiety (MM) and a cleavable moiety (CM). The prodomain may include other sequence elements, such as, for example, a spacer, one or more linkers (e.g., positioned between an MM and a CM and/or between an MM and a VL and/or between an MM and a VH, and/or between a CM and a VL, and/or between a CM and a VH, and the like), and the like.

The terms “masking moiety” and “MM”, are used interchangeably herein to refer to a peptide that, when positioned proximal to the AB, interferes with binding of the AB to the biological target.

The terms “cleavable moiety” and “CM” are used interchangeably herein to refer to a peptide that comprises a substrate for at least one protease.

The terms “clipped impurity,” and “clipped variant” are used interchangeably herein to the molecule that results after protease-mediated cleavage of an intact activatable antibody. A clipped impurity comprises the AB of the corresponding activatable antibody, but lacks all or a portion of the MM (and thus lacks all, or a portion of the prodomain, e.g., lacks all or a portion of at least one CM and lacks all of the corresponding MM). The terms “clipped impurity” and “clipped variant” may include both “single-arm clipped” and “fully clipped” species.

As used herein, the term “specific binding affinity” refers to a preferential binding of an AB to a particular biological target.

The term “single-arm clipped impurity” or “single-arm clipped species” are used interchangeably herein to refer to a clipped impurity in which all or a portion of only one prodomain of the corresponding intact activatable antibody is missing. The term “single-arm clipped” is used in connection with variants of activatable antibodies, wherein the intact activatable antibody comprises two or more antigen binding domains (AB) and two or more prodomains.

The term “fully clipped impurity” or “fully clipped species” are used interchangeably herein to refer to a clipped impurity in which all or a portion of each prodomain of the corresponding intact activatable antibody is missing.

As used herein, the terms “peptide”, “polypeptide”, and “protein” are used interchangeably to refer to a polymer or oligomer comprising naturally occurring or non-naturally occurring amino acid residues or amino acid analogues.

The terms “percent clipped impurity,” “% clipped impurity,” “percent clipped prodomain-encoding polypeptide,” and “% clipped prodomain-encoding polypeptide” are used interchangeably herein to refer to the relative quantity of clipped prodomain-encoding polypeptide present in a composition as a percentage of the total of clipped and intact prodomain-encoding polypeptide, where quantities are determined by reducing SDS-capillary gel electrophoresis (“reducing SDS-cGE”). A reducing SDS-cGE assay is illustrated in Example 1. Percent clipped impurity is computed on a relative percent peak area basis for clipped prodomain-encoding polypeptide and intact prodomain-encoding polypeptide as follows:

$$\% clipped impurity = \frac{\% Peak Area {,}_{clipped}}{( \% Peak Area {,}_{clipped} + \% Peak Area {,}_{intact} )} \times 100$$

$$where :$$

$$\% Peak Area {,}_{clipped} = \frac{( Peak Area of clipped prodomain - encoding polypeptide )}{( Total Peak Area corresponding to all species detected )} \times 100$$

$$\% Peak Area {,}_{intact} = \frac{( Peak Area of intact prodomain - encoding polypeptide )}{( Total Peak Area corresponding to all species detected )} \times 100$$

The Total Peak Area corresponding to all species detected refers to the sum of all peak areas in the SDS-cGE chromatograph.

The terms “percent intact activatable antibody,” “% intact activatable antibody,” “percent intact prodomain-encoding polypeptide,” and “% intact prodomain-encoding polypeptide” are used interchangeably herein to refer to the relative quantity of intact prodomain-encoding polypeptide present in a composition as a percentage of the total of clipped and intact prodomain-encoding polypeptide, where quantities are determined by reducing SDS-cGE, such as, for example the reducing SDS-cGE assay illustrated in Example 1. Percent intact activatable antibody is computed on the basis of relative percent peak area for clipped prodomain-encoding polypeptide and intact prodomain-encoding polypeptide as follows:

$$\% intact activatable antibody = \frac{\% Peak Area {,}_{intact}}{( \% Peak Area {,}_{clipped} + \% Peak Area {,}_{intact} )} \times 100$$

$$where :$$

$$\% Peak Area {,}_{clipped} = \frac{( Peak Area of clipped prodomain - encoding polypeptide )}{( Total Peak Area corresponding to all species detected )} \times 100$$

$$\% Peak Area {,}_{intact} = \frac{( Peak Area of intact prodomain - encoding polypeptide )}{( Total Peak Area corresponding to all species detected )} \times 100$$

The term “prodomain-encoding polypeptide” refers to a polypeptide in an activatable antibody that contains the amino acid sequence which encodes a prodomain. Prodomain-encoding polypeptides may contain amino acid sequence(s) that encode other elements of an activatable antibody in addition to a prodomain. For example, if the prodomain resides within a polypeptide encoding an antibody light chain, the prodomain-encoding polypeptide also encodes at least a VL. Likewise, in other embodiments, if the prodomain resides within a polypeptide encoding an antibody heavy chain, the prodomain-encoding polypeptide also encodes at least a VH. Similarly, if the prodomain resides within a polypeptide encoding an scFv chain, the prodomain-encoding polypeptide further encodes at least a VL and a VH, as well.

The term “clipped prodomain-encoding polypeptide” refers herein to the truncated prodomain-encoding polypeptide that results after the prodomain has been clipped.

The term “intact prodomain-encoding polypeptide” refers herein to a prodomain-encoding polypeptide that has not been clipped, and which contains an intact prodomain.

The term “substantially depleted” is used herein to refer to an eluate composition having either no detectable clipped impurity present, or a relative level of clipped impurity that has been reduced by at least 20%, as compared to the level of clipped impurity present in the aqueous feedstock, where percent reduction is computed as follows:

$$\frac{\begin{matrix}{( {}^{‶} \% clipped {impurity}^{″} )}_{aqueous feedstock} - \\ {( {}^{‶} \% clipped {impurity}^{″} )}_{eluate}\end{matrix}}{{( {}^{‶} \% clipped {impurity}^{″} )}_{aqueous feedstock}} \times 100 ,$$

where “% clipped impurity” is the same as defined above.

The term “substantially free” is used herein in connection with clipped variant and clipped impurity to refer to a composition having either no detectable clipped impurity present, or having clipped impurity present wherein the % clipped impurity is less than 5% as determined by SDS-cGE, e.g., 4%, 3%, 2%, 1%, 0.5%, 0.1%, 0.05% or any numeric value or range between zero and 5%.

The term “bound corresponding clipped impurity” and “bound clipped impurity” is used interchangeably herein to refer to clipped impurity that is retained in a chromatography column.

The term “spacer” refers herein to an amino acid residue or a peptide incorporated at a free terminus of the prodomain. In some aspects, a spacer (or “header”) may contain glutamine (Q) residues. In some aspects, residues in the spacer minimize aminopeptidase and/or exopeptidase action to prevent cleavage of N-terminal amino acids.

The term “linker” refers herein to an amino acid residue or a peptide that functions to provide further physical separation between the MM, CM, and/or AB elements of the activatable antibody.

As used herein, the terms “high molecular weight species” and “HMWS” are used interchangeably herein to refer to composition impurities (e.g., aggregates) having an effective molecular weight that is greater than that of monomeric intact activatable antibody, as determined by a size exclusion high performance liquid chromatography (SE-HPLC, such as the assay described in Example 1).

The term “effective molecular weight” refers to the molecular weight as determined by an SE-HPLC assay, such as the assay described in Example 1.

As used herein, the term “isocratic” refers to the use of an eluent having a substantially constant, or fixed composition, for the duration of the elution step.

As used herein, the term “hydrophobic interaction chromatography stationary phase” or “HIC stationary phase” refers interchangeably to a type of stationary phase that has ligands which are designed to interact with compounds by exclusively hydrophobic interactions.

The term “multimodal chromatography stationary phase” or “MMC stationary phase” refers interchangeably herein to a type of stationary phase that has ligands which interact with compounds via hydrophobic interactions, as well as one or more additional type of interaction that is not a hydrophobic interaction (such as, for example, electrostatic (via charged ligand substituents), hydrogen bonding, thiophilicity and the like).

As used herein, the term “host cell proteins” and “HCP” are used interchangeably herein to refer to proteins that are native to the host cell from which intact activatable antibody is expressed.

As used herein, the term “total protein yield” refers to the percentage of total protein recovered in a chromatography eluate on the basis of total protein in the aqueous feedstock, and as measured by absorbance at a wavelength of 280 nm in a UV spectrometry assay.

As used herein, the term “intervening unit operation” refers to a process step that occurs between the cell culturing process step and the hydrophobic chromatography process step.

The term “bulk intermediate product composition” refers to the composition of the product of an intervening unit operation.

As used herein, the term “bioharvest composition” refers to a composition derived from a cell culturing step that produces a composition comprising an activatable antibody of interest.

The term “serial”, when used in connection with describing the relationship between unit operations, is used herein to refer to two or more unit operations being performed in sequence with respect to time and order, either batch-wise, semi-continuously, or continuously, as a first, then a second, and so on.

As used herein, the term “conjugation reagent” refers to a reactive form of a conjugation moiety that comprises a conjugation moiety covalently bound to a reactive linker.

As used herein, the term “corresponds substantially” when used in connection with two amino acid sequences refers to a level of identity of at least about 90% when optimally aligned.

Two polypeptide sequences are “optimally aligned” when they are aligned using defined parameters, i.e., a defined amino acid substitution matrix, gap existence penalty (also termed gap open penalty), and a gap extension penalty, so as to arrive at the highest similarity score possible for that pair of sequences. The BLOSUM62 matrix (Henikoff and Henikoff (1992) Proc. Natl. Acad. Sci. USA 89 (22): 10915-10919) is often used as a default scoring substitution matrix in polypeptide sequence alignment algorithms (such as BLASTP). The gap existence penalty is imposed for the introduction of a single amino acid gap in one of the aligned sequences, and the gap extension penalty is imposed for each residue position in the gap. Unless otherwise stated, alignment parameters employed herein are: BLOSSUM62 scoring matrix, gap existence penalty-11, and a gap extension penalty-1. The alignment score is defined by the amino acid positions of each sequence at which the alignment begins and ends (e.g., the alignment window), and optionally by the insertion of a gap or multiple gaps into one or both sequences, so as to arrive at the highest possible similarity score.

With respect to any and all numerical ranges provided herein, the ranges are intended to be inclusive of the numerical limits that define the range.

### Processes and Compositions of the Present Invention

The present invention relates to the production of purified compositions of intact protease-activatable antibody that are substantially free of clipped impurity and processes for producing such compositions. The process for making the purified compositions is also effective at removing other impurities. Structurally, activatable antibodies comprise: (1) an antigen binding domain (AB) that, when not masked, specifically binds a biological target; and (2) a prodomain coupled to the AB that comprises a masking moiety (MM) and a cleavable moiety (CM). The CM is positioned relative to the MM and AB components such that cleavage of the CM results in the untethering of the MM from its position proximal to the AB. Cleavage thus typically results in generation of an activated antibody that is capable of specifically binding the biological target. Often, the activatable antibody is a homodimer that comprises two identical AB, two identical CM, and two identical MM. In such instances, a clipped variant of the activatable antibody may be a single-arm clipped variant in which only one of the two CM is cleaved, so that the clipped variant comprises one complete CM and one complete MM, but is missing a portion of one CM and all of one MM.

Activatable antibodies may be designed to selectively activate in diseased tissue by incorporating within the CM, a substrate for a protease more prevalently found in an active state in the diseased tissue. Activatable antibodies thus have the potential to mitigate target-mediated toxicity that may arise when monoclonal antibodies bind a biological target that is widely distributed beyond the site of disease. See Desnoyers, et al., Science Translational Medicine (16 Oct. 2013)5 (207): 207ra144. Activatable antibodies are described in a number of publications, including, for example, WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740, U.S. Provisional Application Ser. Nos. 62/469,429, 62/572,467, and 62/613,358, WO 2012/025525, WO 2017/025698, WO 2016/046778, WO 2016/179003, WO 2016/182064, WO 2017/156178, WO 2017/143094, WO 2017/162587, WO 2013/128194, each of which is incorporated herein by reference in their entireties. Further illustrative examples of activatable antibodies suitable for use in the processes and compositions of the present invention are described in more detail hereinbelow.

Though a highly desired product, the manufacture of purified compositions of activatable antibodies is made more challenging due to their activatable nature. Analysis of activatable antibody bioharvest revealed the presence of clipped impurity—a compound that is uniquely associated with activatable antibodies. Due to the potential effects of off-target toxicity and decreased efficacy, it is undesirable to have significant amounts of the clipped impurity, i.e., “activated” activatable antibody that can freely bind its biological target, in a composition or pharmaceutical product. The presence of clipped variants in drug product is undesired because of the potential to impact the safety profile of the product.

Removal of clipped impurity from intact activatable antibody compositions is made challenging due to the relatively small differences between desired product and clipped impurity with respect to properties such as molecular size, structure, amino acid composition, and the fact that their amino acid sequences may be 100% identical over a majority of their respective structures. Structurally, the difference between an intact activatable antibody and the corresponding clipped impurity is the absence of all or a portion of at least one prodomain in the clipped impurity. In some embodiments, the masking moiety is a relatively short peptide sequence, for example less than 50 amino acids, less than 40, 30, 20, 15, 14, 13, 12, 11, or 10 amino acids long, and the clipped species and the intact activatable antibody may differ by approximately the number of amino acids in the masking moiety. Further, the inventors have discovered that single-arm clipped variants are the predominant clipped variant, such that the entire difference between an intact activatable antibody and a clipped variant is the absence of a portion of a single prodomain, for example the clipped variant differs from the intact activatable antibody only in that lacks a MM and a portion of a CM. Thus, the size and physicochemical properties of the intact activatable antibody and a clipped variant thereof are similar, making separation of the molecules difficult.

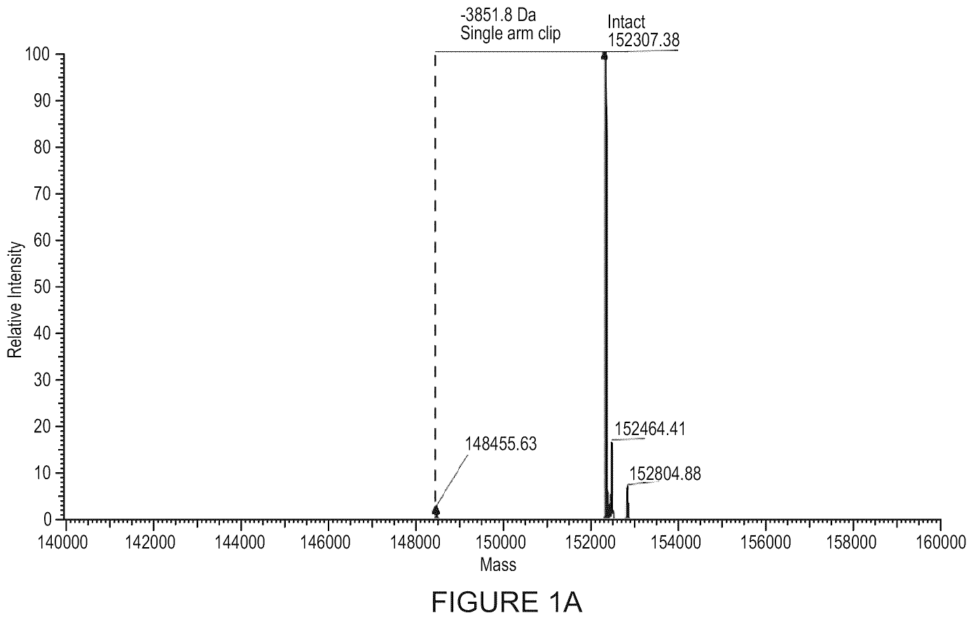

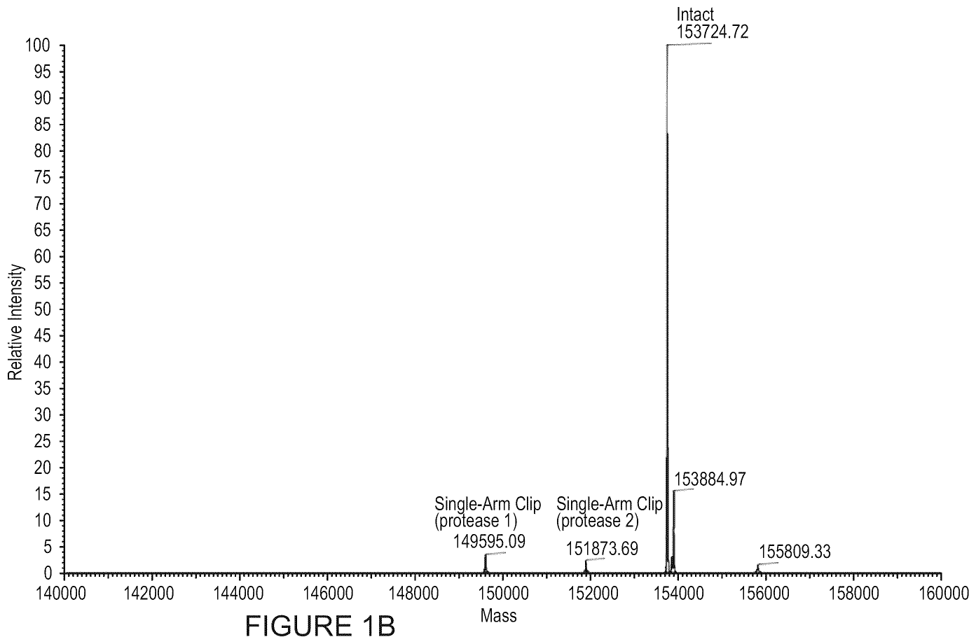

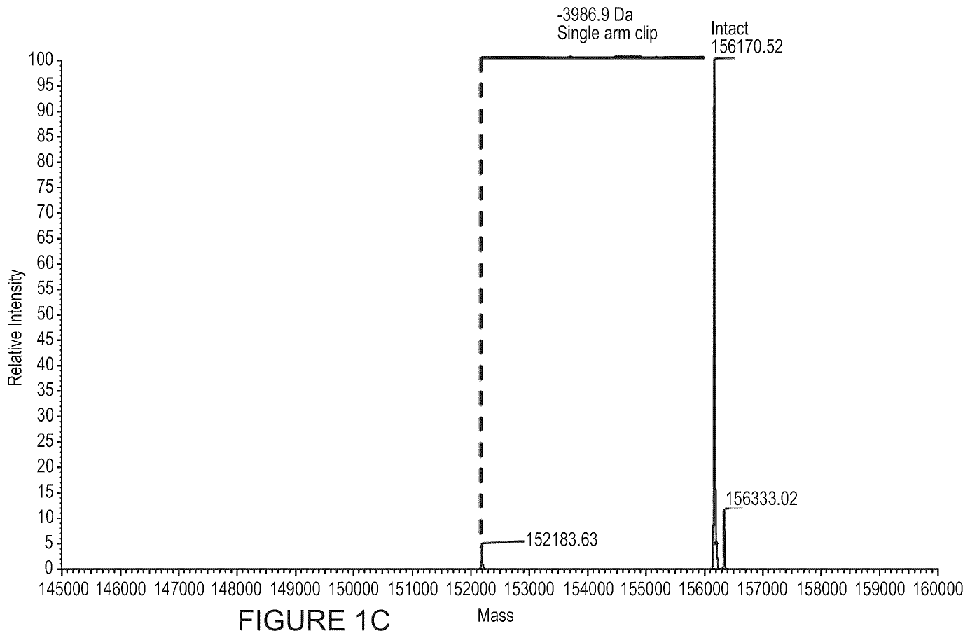

Characterization of bioharvest compositions comprising activatable antibody having two light chains, each encoding a prodomain, and two heavy chains, revealed that all, or nearly all of the clipped impurity appears to be single-arm clipped impurity (i.e., not fully clipped impurity), as shown by the mass spectrums of three different activatable antibody compositions in FIGS. 1A-C. Thus, when an activatable antibody has more than one prodomain-encoding polypeptide, separation of intact activatable antibody and clipped impurity is exacerbated by the fact that the predominant clipped species, single-arm clipped impurity, is structurally very similar to that of the corresponding intact activatable antibody. In some aspects, the molecular weight of the clipped impurity is about 93, 94, 95, 96, 97, 98, or 99% of the molecular weight the intact activatable antibody. In some aspects, the molecular weight of the clipped prodomain-encoding polypeptide is about 93, 94, 95, 96, 97, 98, or 99% of the molecular weight of the intact prodomain-encoding polypeptide. In some aspects, the clipped impurity is about 93, 94, 95, 96, 97, 98, or 99% identical to the amino acid sequence of the intact activatable antibody.

In some aspects, a clipped variant may include 1, 2, 3, 4, or more amino acid residues of the prodomain. In some aspects, a clipped variant may include 1, 2, 3, 4, or more amino acid residues of the CM. In some aspects, a clipped variant may include 1, 2, 3, 4, or more amino acid residues of a linker. In some aspects, a clipped variant may include 1, 2, 3, 4, or more amino acid residues of a linker and the CM.

Despite this challenge, a process has been discovered that is remarkably effective at separating intact activatable antibody from corresponding clipped impurity at relatively high yield. The process for producing purified compositions of intact activatable antibody comprises:
(a) loading an aqueous feedstock comprising water, an intact activatable antibody, a clipped impurity, and a first salt onto a chromatography column,
wherein the chromatography column comprises a stationary phase that comprises a support matrix and ligands bound thereto,
   wherein the ligands comprise a hydrophobic substituent, and
wherein the intact activatable antibody comprises (i) at least a first antigen binding domain (AB) that has a specific binding affinity for a first biological target, and (ii) a first prodomain,
   wherein the at least first AB comprises a first antibody light variable domain (VL) and a first antibody heavy variable domain (VH),
   wherein the first prodomain comprises a first masking moiety (MM) and a first cleavable moiety (CM), and
   wherein the first AB is coupled to the first prodomain; and
(b) eluting the chromatography column with an eluent comprising water and a second salt to generate an eluate that comprises a purified composition comprising intact activatable antibody,
wherein the eluate is substantially depleted of the clipped impurity.

Steps (a) and (b) are collectively referred to herein as the “hydrophobic chromatography process”. The eluate, and thus the purified composition, are both substantially depleted of the clipped impurity. Distinct and separate peaks of intact activatable antibody and corresponding clipped impurity were observed in elution product from the chromatography column. Despite the similarity in structure between intact and clipped impurity, the compounds were readily separable on chromatography columns that relied, at least in part, on hydrophobic interactions. The intact activatable antibody-rich eluate that was generated was substantially depleted of the corresponding clipped impurity. The process is a readily scalable and highly productive process for generating highly pure compositions of intact activatable antibody at high total protein yields.

It was discovered that intact activatable antibody elutes in a distinct peak prior to the corresponding clipped impurity, and also, prior to other impurities, such as, for example, high molecular weight species (“HMWS”) and host cell protein (HCP). This phenomenon was observed across a variety of aqueous feedstock compositions comprising intact activatable antibody and corresponding clipped impurity having different amino acid sequences and specificities to different biological targets, as demonstrated in the Examples. In view of the resolution and peak profiles that were achieved, in some embodiments, elution step (b) is carried out under isocratic conditions.

In many embodiments, after performing step (b), the process further comprises a column cleaning step that comprises washing the chromatography column with a cleaning agent. Often in these embodiments, the process of the present invention does not comprise a step of eluting bound corresponding clipped impurity and/or other impurities if present in the aqueous feedstock (such as, for example, HMWS, HCP, and the like) from the chromatography column with a second eluent prior to the column cleaning step. Because the impurities are substantially retained on the column after elution of the intact activatable antibody, it may be desired to carry out the cleaning step without separately eluting the impurities, thus further enhancing the highly productive nature of the process of the present invention. Suitable cleaning agents include any of a wide variety known in the art, including for example, an aqueous acid solution; an aqueous base solution, an organic solvent, a mixed organic solvent (e.g., comprising two or more different organic solvents), an aqueous mixture of one or more organic solvents, a mixture of any of the foregoing, and the like. Illustrative cleaning agents include an aqueous sodium hydroxide solution, ethanol, isopropanol, ethylene glycol, a guanidine hydrochloride solution, an acidic pepsin solution, a sodium lauroyl sarcosinate solution, and the like, as well as any combination of two or more thereof.

With regards to the stationary phase employed in the process of the present invention, any of a wide variety of known support matrix materials may be used. Illustrative support matrix materials that are suitable include a hydrophilic polymer, such as, for example, a carbohydrate (such as, for example, agarose (e.g., SEPHAROSE brand (GE Healthcare Lifesciences), Capto™ ImpRes (GE Healthcare Lifesciences), a cross-linked cellulose (e.g., Cellufine™ HIC media (Amsbio), and the like), a polymethacrylate-based resin (e.g., Macro-Prep® HIC resin (Bio-Rad, Inc.), and the like), a polystyrene-based resin (e.g., Bio-Beads™ SM-2 Resin (Bio-Rad, Inc.), and the like), silica, a synthetic co-polymer, as well as any other chromatography support matrix materials that are known in the art. The support matrix may be in any of a variety of forms, including, for example, particulate form, bead form, membrane form, and the like.

Any of a variety of known ligand species having a hydrophobic substituent may be employed in the processes of the present invention. Exemplary hydrophobic substituents include, for example, a straight chain alkyl substituent (including, for example, methyl, ethyl, n-propyl, n-butyl, n-pentyl, n-hexyl, n-heptyl, n-octyl, and the like), a branched alkyl substituent (such as, for example, i-propyl, t-butyl, and the like), an aryl substituent (such as, for example, phenyl, an alkyl-substituted phenyl, and the like), and the like, as well as any combination of two or more types of hydrophobic substituents. In some embodiments, the hydrophobic substituent comprises a C₄ to C₁₀ alkyl substituent (i.e., a C₄, C₅, C₆, C₇, C₈, C₉, or a C₁₀ substituent) and/or a phenyl substituent. Often, the C₄ to C₁₀ alkyl substituent is selected from the group consisting of a butyl substituent (i.e., C₄) or an octyl substituent (i.e., C₈). The hydrophobic substituents may be coupled to the support matrix via any of a variety of linkages, including, for example, O-ether, S-ether, and the like. Exemplary ligands (including-O-linker) that are suitable for use in the practice of the present invention include —O-Ph, —S—(CH₂)₃—CH₃ (—S-butyl), —O—(CH₂)₃—CH₃ (O-butyl), —O—(CH₂)₇—CH₃ (O-octyl), —O—CH₂—CHOH—CH₂—OH, —O—CH—(CH₃)₂, and the like. In certain embodiments, as described in more detail hereinbelow, the ligands may further comprise additional substituents that facilitate separation by interactions other than hydrophobic, such as, for example, electrostatic, hydrogen bonding, thiophilic, and the like.

In some embodiments, the stationary phase is a hydrophobic interaction chromatography (HIC) stationary phase that facilitates separation by hydrophobic interactions only. Exemplary HIC stationary phase ligands may comprise any of the hydrophobic substituents described hereinabove. In certain embodiments, the ligands comprise a substituent selected from the group consisting of phenyl, butyl, octyl, and isopropyl. HIC stationary phases are readily available commercially. In specific embodiments, the stationary phase comprises a ligand selected from the group consisting of —O-phenyl, —S—(CH₂)₃—CH₃ (i.e., —S-butyl), —O—(CH₂)₃—CH₃ (i.e., O-butyl), —O—(CH₂)₇—CH₃ (i.e., O-octyl), —O—CH₂—CHOH—CH₂—OH, —O—CH—(CH₃)₂, and the like, as well as any combination of two or more different ligands.

In other embodiments, the stationary phase is a multimodal chromatography (MMC) stationary phase. In these embodiments, the ligands comprise one or more hydrophobic substituents, and at least one further substituent that facilitates separation on the basis of an interaction other than hydrophobicity, such as, for example, electrostatic, hydrogen bonding, thiophilicity, and the like. Illustrative MMC stationary phases that are suitable for use in the practice of the present invention comprise ligands that have, for example, a hydrophobic substituent and one or more substituents selected from the group consisting of a sulfide substituent, a carboxyl substituent, and an amine substituent. Often, the carboxyl substituent and/or amine substituent are charged under the process conditions employed. MMC stationary phases that are suitable for use in the practice of the present invention include those having a ligand selected from the group consisting of N-benzyl methyl ethanolamine, N-benzyl methyl ethanolamine, N-benzoyl-homocysteine, N-benzoyl-homocysteine, octylamine, and the like.

The presence of a hydrophobic substituent in the immobilized ligand appeared to have a substantial impact on facilitating the separation of intact activatable antibody from clipped impurity. Separation of intact activatable antibody from clipped impurity was achieved to a high degree, using the hydrophobic chromatography process as illustrated in Examples 3 (using a HIC stationary phase), 4 (using a HIC stationary phase), and 5 (using an MMC stationary phase). In contrast, separation of intact activatable antibody from clipped impurity was not achieved using cation exchange chromatography, as described in Example 2. Likewise, the use of anion chromatography similarly did not appear to have an impact on separating intact activatable antibody from clipped impurity, as described in Examples 3-5.

The hydrophobic chromatography process is initiated by loading onto the chromatography column an aqueous feedstock comprising water, intact activatable antibody, clipped impurity, and a first salt. In some embodiments, the aqueous feedstock may comprise additional components, including additional impurities, such as, for example, one or more products of a cell culturing process step (e.g., host cell proteins, DNA, and the like), one or more residual compounds from an upstream purification unit operation (e.g., Protein A, Protein G, and the like), HMWS (e.g., aggregates of monomeric activatable antibody species present in the aqueous feedstock, and the like), low molecular weight species (LMWS), and the like.

Salts suitable for use as a first salt in the aqueous feedstock may be any salt that promotes binding of intact activatable antibody and clipped impurity to the stationary phase of the column. Suitable first salts and first salt concentrations for the aqueous feedstock and second salts and second salt concentrations for the eluent can be readily determined by performing a series of test runs using a gradient of salt concentration and identifying the salt and salt concentration that effects binding of intact activatable antibody to the stationary phase while allowing impurities to either wash through the stationary phase or remain bound to the stationary phase, and identifying a second salt and salt concentration that effects selective elution of the desired intact activatable antibody from the stationary phase. In some embodiments, the first salt and the second salt are the same salt. In other embodiments, the first salt and the second salt are different salts. The first and/or second salt may each independently also comprise a mixture of salt species.

Illustrative first salts and second salts each independently comprise an anion selected from the group consisting of PO₄³⁻, SO₄²⁻, OH⁻, HPO₄²⁻, CH₃COO— (acetate ion), citrate ion, F⁻, Cl⁻, Br⁻, H₂PO₄⁻, I⁻, NO₃⁻, ClO₄⁻, SCN⁻, an amino acid anion, and the like. In some embodiments, the first salt and the second salt may each independently comprise a cation selected from the group consisting of N(CH₃)₄⁺, NH₄⁺, Cs⁺, Rb⁺, K⁺, Na⁺, H⁺, Ca⁺, Mg²⁺, Al³⁺, an amino acid cation, and the like. The first salt may be introduced into an aqueous composition comprising the intact activatable antibody and the corresponding clipped impurity just prior to the hydrophobic chromatography process step, or it may be introduced in connection with a process step upstream of the hydrophobic chromatography process step (e.g., in an intervening unit operation as described hereinbelow). In this latter situation, additional first salt may be optionally added to the composition and/or the composition may be diluted to accommodate desired load conditions for the column.

When the chromatography column comprises a HIC stationary phase, the first salt typically exhibits kosmotropic (salting out) behavior. Such salts can be readily identified, for example, from the Hofmeister series of ions. Sec, e.g., Tadeo, et al., Biophysical Journal (2009) 97:2595 and Hyde, et al., Org. Process Res. Dev. (2017)21:1355, both of which are incorporated herein by reference in their entireties. In some embodiments, the first salt and the second salt each independently comprise an anion and/or cation that is more strongly kosmotropic than chaotropic. In certain of these embodiments, the first salt and the second salt each independently comprise an anion selected from the group consisting of PO₄³⁻, SO₄²⁻, OH⁻, HPO₄²⁻, F⁻, CH₃COO⁻ (acetate ion), citrate ion, an amino acid anion, and Cl⁻. In other embodiments, the first salt and the second salt each independently comprise an anion selected from the group consisting of PO₄³⁻, SO₄²⁻ and HPO₄²⁻.

In certain of these embodiments, the first salt and the second salt each independently comprise a cation selected from the group consisting of N(CH₃)₄⁺, NH₄⁺, Cs⁺, Rb⁺, K⁺, Na⁺, H⁺, Ca⁺, Mg²⁺, Al³⁺, and an amino acid cation. In some embodiments, the first salt and the second salt each independently comprise a cation selected from the group consisting of NH₄⁺, K⁺, Na⁺, Li⁺, and Mg²⁺. In certain embodiments, the first salt and the second salt each independently comprise a cation selected from the group consisting of NH₄⁺, K⁺, and Na⁺. In some embodiments, the first salt and the second salt each independently comprise a cation selected from the group consisting of NH₄⁺, K⁺, and Na⁺, and an anion selected from the group consisting of PO₄³⁻, SO₄²⁻, OH⁻, HPO₄²⁻, CH₃COO— (acetate ion), citrate ion, F⁻, Cl⁻, Br⁻, H₂PO₄⁻, I⁻, NO₃⁻, ClO₄⁻, and SCN⁻.

In some embodiments, the first salt and the second salt are each independently selected from the group consisting of (NH₄)₂SO₄, Na₂SO₄, Na₃PO₄, K₃PO₄, NaCl, KCl, and CH₃COONH₄. In certain embodiments, the first salt and the second salt are each independently selected from the group consisting of (NH₄)₂SO₄, Na₂SO₄, Na₃PO₄ and K₃PO₄. In other embodiments, the first salt and the second salt are each independently selected from the group consisting of (NH₄)₂SO₄ and Na₂SO₄. In certain embodiments, at least one of the first salt and the second salt comprises (NH₄)₂SO₄. In some of these embodiments, both the first salt and the second salt comprise (NH₄)₂SO₄. In other embodiments, at least one of the first salt and the second salt comprises Na₂SO₄. In some of these embodiments, both the first salt and the second salt comprise Na₂SO₄.

When the chromatography column comprises a HIC stationary phase, the eluent is generally less polar than the aqueous feedstock. In some embodiments, the first salt concentration (i.e., in the aqueous feedstock) is greater than the second salt concentration (i.e., in the eluent). For example, the first salt concentration may be about 1.5× greater, about 2× greater, about 3× greater, about 4× greater, about 5× greater, about 6× greater, about 7× greater, about 8× greater, about 9× greater, or about 10× greater than the second salt concentration. For example, the first salt may comprise about 1.5 M ammonium sulfate and the second salt may comprise about 0.25 M ammonium sulfate (about 6× greater concentration of the first salt than the second salt). When it is desired to use a second salt that is different from the first salt, the second salt is typically lower in kosmotropic strength as compared to the first salt. Cations and anions having greater or lesser kosmotropic strength can be readily identified in accordance with the Hofmeister series of ionic strength. See, e.g., Tadeo, et al., Biophysical Journal (2009)97:2595 and Hyde, et al., Org. Process Res. Dev. (2017)21:1355, both of which are incorporated herein by reference in their entireties. In some embodiments, the eluent further comprises a water miscible organic solvent, such as, for example, an alcohol, a diol, a polyol, and the like.

When the chromatography column comprises an MMC stationary phase, the first salt typically exhibits chaotropic (salting in) behavior. As with the salts employed in connection with a HIC stationary phase, salts suitable for use in connection with an MMC-based process can also be readily identified from the Hofmeister series of ions. In some embodiments, the first and second salt each independently comprise an anion and/or cation that is more strongly chaotropic than kosmotropic. Sec, e.g., Tadeo, et al., Biophysical Journal (2009)97:2595 and Hyde, et al., Org. Process Res. Dev. (2017)21:1355, both of which are incorporated herein by reference in their entireties. In certain of these embodiments, the first salt and the second salt each independently comprise an anion selected from the group consisting of Cl⁻, Br⁻, H₂PO₄⁻, I⁻, NO₃⁻, ClO₄⁻, an amino acid anion, and SCN⁻. In some of these embodiments, the first salt and the second salt each independently comprise an anion selected from the group consisting of Cl⁻, Br⁻, H₂PO₄⁻, I⁻, NO₃⁻, an amino acid anion, and ClO₄⁻, and SCN⁻.

In certain embodiments, the first salt and the second salt each independently comprise a cation selected from the group consisting of N(CH₃)₄⁺, NH₄⁺, Ba⁺, Ca²⁺, Mg²⁺, Cs⁺, Rb⁺, K⁺, Na⁺, and an amino acid cation. In some embodiments, the cation is selected from the group consisting of N(CH₃)₄⁺, NH₄⁺, Ca²⁺, Mg²⁺, K⁺, Na⁺, and an amino acid cation. When an amino acid cation is employed, it is often an arginine cation (e.g., with a positively charged guanidino moiety).

Illustrative first salts include, for example, arginine chloride or arginine hydrochloride, NaCl, and the like. When the chromatography column comprises an MMC stationary phase, the first salt and the second salt are often the same salt. In these embodiments, the eluent typically comprises a concentration of the salt that is greater than the concentration of the salt in the aqueous feedstock. For example, the eluent (second salt) salt concentration may be about 1.5× greater, about 2× greater, about 3× greater, about 4× greater, about 5× greater, about 6× greater, about 7× greater, about 8× greater, about 9× greater, or about 10× greater than the feedstock (first salt) salt concentration. When the first salt and the second salt are different, a second salt is often selected that is higher in chaotropic strength as compared to the first salt. Cations and anions have greater or lesser chaotropic strength can be readily identified in accordance with the Hofmeister series of ionic strength. See, e.g., Tadeo, et al., Biophysical Journal (2009)97:2595 and Hyde, et al., Org. Process Res. Dev. (2017)21:1355, both of which are incorporated herein by reference in their entireties. In certain embodiments where the first salt and the second salt are different, the eluent comprises both the first salt and the second salt. In some aspects, the first salt may comprise about 30 mM sodium chloride as the only salt, and the second salt may comprise about 30 mM sodium chloride and about 90 mM arginine hydrochloride.

The aqueous feedstock and eluent may each independently also comprise one or more buffering agents. Suitable buffering agents include one or more salts, such as, for example, any of those listed hereinabove as a suitable first salt; an acid, such as, for example, 2-(N-morpholino) ethanesulfonic acid (MES), (3-(N-morpholino) propanesulfonic acid) (MOPS), (4-(2-hydroxyethyl)-1-piperazineethanesulfonic acid) (HEPES), hydrogen chloride, and the like; and/or a base, such as, for example, sodium hydroxide, potassium hydroxide, and the like.

In certain embodiments, the clipped impurity consists of single-arm clipped impurity. In some embodiments in which the intact activatable antibody comprises multiple ABs and correspondingly, multiple prodomains, the clipped impurity may comprise a mixture of clipped impurity species. In some of these embodiments, the mixture of clipped impurity species comprises single-arm clipped impurity and fully clipped impurity. In still other embodiments in which the intact activatable antibody comprises multiple ABs and correspondingly, multiple prodomains, the clipped impurity consists essentially of single-arm clipped impurity. In some of these embodiments, the clipped species consists of single-arm clipped impurity. Single-arm clipped impurity and fully clipped impurity can be readily determined by mass spectrometry.

In some embodiments, the aqueous feedstock comprises a pH in the range of from about 5.0 to about 8.0, or from about 5.0 to about 7.5, or from about 5.0 to about 7.0, or from about 5.5 to about 6.5, or from about 5.7 to 6.3, or from about 5.8 to 6.2, or from about 5.6 to about 6.0. The temperature at which the loading and eluting steps are carried out may be the same or different. In some embodiments, the loading and eluting steps are each independently carried out at a temperature in the range of from about 10° C. to about 30° C., or in the range of from about 15° C. to about 30° C., or in the range of from about 15° C. to about 29° C., or in the range of from about 15° C. to about 28° C., or in the range of from about 15° C. to about 27° C., or in the range of from about 15° C. to about 26° C., or in the range of from about 15° C. to about 25° C., or in the range of from about 16° C. to about 25° C., or in the range of from about 17° C. to about 25° C., or in the range of from about 18° C. to about 25° C. Typically, the loading and eluting steps are carried out at a temperature in the same temperature range. Often the target temperature is about the same for both the loading and eluting steps. In some embodiments, it may be desired to carry out the eluting step at a temperature that is different from the temperature at which the loading step is carried out. In certain embodiments, it may be desired to carry out the process steps at a temperature that is higher or lower than the endpoints of the ranges described hereinabove. For example, when a HIC stationary phase is employed, a higher temperature employed during the loading step may increase hydrophobic interactions with the column and a lower temperature during the eluting step may encourage release of components from the column by decreasing hydrophobic interactions with the column.

The chromatography column may be pre-conditioned to have a pH, first salt concentration, and temperature that is about the same as that of the aqueous feedstock. This may be accomplished by loading sufficient quantities (e.g., one or more column volumes) of buffer having the same pH, first salt concentration, and/or temperature as the aqueous feedstock into the chromatography column until target conditions are achieved in the column.

After the loading step, the column may be washed with a washing buffer to remove non-binding components from the column before the elution step. In some embodiments, the washing buffer comprises a salt at about the same concentration or a concentration that is higher than the concentration of first salt in the aqueous feedstock and is often at about the same pH as the aqueous feedstock.

It has been discovered that aqueous feedstocks comprising intact activatable antibody and impurities can be purified at high total protein yields using the processes described herein. In some embodiments, the yield of total protein in the eluate is at least about 60%, at least about 65%, at least about 70%, or at least about 71%, or at least about 72%, or at least about 73%, or at least about 74%, or at least about 75%, or at least about 76%, or at last about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, as determined by absorbance at a wavelength of 280 nm. An illustrative absorbance assay for determining total protein is described in Example 1, herein below.

The hydrophobic chromatography process is highly effective at removing clipped impurity from the process stream (i.e., collectively, the stream of aqueous feedstock into the hydrophobic process and the stream of eluant out of the hydrophobic process). In some embodiments, the level of reduction in relative quantity of clipped impurity in the aqueous feedstock compared to the relative quantity of clipped impurity in the eluate is at least about 2-fold, at least about 3-fold, at least about 4-fold, at least about 5-fold, at least about 6-fold, at least about 7-fold, or at least about 8-fold, or at least about 9-fold, or at least 10-fold, or at least about 15-fold, or at least about 20-fold, as measured by reducing SDS-capillary gel electrophoresis (SDS-cGE). In some embodiments, no clipped impurity is detected in the eluate. The term “relative quantity of clipped impurity” refers herein to “% clipped impurity” is as defined hereinbelow.

As used herein, the phrase “level of reduction” or “reducing” (and grammatical variants thereof), when used in connection with an impurity, such as clipped impurity, HCP, HWMS, and the like, refers to the extent of reduction in quantity of the impurity, as determined by comparing the quantity of impurity in the aqueous feedstock to the quantity of impurity in the eluate. The level of reduction in impurity can be represented in terms of a ratio (or equivalently, as a fold reduction) or in terms of a percent reduction.

When clipped impurity is present in the eluate, the fold reduction in level of clipped impurity is determined by the following formula:

$$Fold Reduction of clipped impurity = \frac{{( \% clipped impurity )}_{aqueous feedstock}}{{( \% clipped impurity )}_{eluate}}$$

where “% clipped impurity” in each of the aqueous feed and eluate is determined according to the following formula:

$${( \% clipped impurity )}_{aqueous feedstock} = \frac{{( \% Peak Area {,}_{clipped} )}_{aqueous feedstock}}{{( \% Peak Area {,}_{clipped} + \% Peak Area {,}_{intact} )}_{aqueous feedstock}} \times 100$$

$$where :$$

$${( \% Peak Area {,}_{clipped} )}_{aqueous feedstock} = \frac{{( Peak Area of clipped prodomain - encoding polypeptide )}_{aqueous feedstock}}{{( Total Peak Area corresponding to all species detected )}_{aqueous feedstock}} \times 100 , and$$

$${( \% Peak Area {,}_{intact} )}_{aqueous feedstock} = \frac{{( Peak Area of intact prodomain - encoding polypeptide )}_{aqueous feedstock}}{{( Total Peak Area corresponding to all species detected )}_{aqueous feedstock}} \times 100 ;$$

$$and ,$$

$${( \% clipped impurity )}_{eluate} = \frac{{( \% Peak Area {,}_{clipped} )}_{eluate}}{{( \% Peak Area {,}_{clipped} + \% Peak Area {,}_{intact} )}_{eluate}} \times 100$$

$$where :$$

$${( \% Peak Area {,}_{clipped} )}_{eluate} = \frac{{( Peak Area of clipped prodomain - encoding polypeptide )}_{eluate}}{{( Total Peak Area corresponding to all species detected )}_{eluate}} \times 100 , and$$

$${( \% Peak Area {,}_{intact} )}_{eluate} = \frac{{( Peak Area of intact prodomain - encoding polypeptide )}_{eluate}}{{( Total Peak Area corresponding to all species detected )}_{eluate}} \times 100.$$

In the above formulae, the clipped impurity is quantified in terms of the polypeptide that is impacted by clipping, i.e., the prodomain-encoding polypeptide. The prodomain-encoding polypeptide may be an antibody light chain, an antibody heavy chain, an scFv, and the like, depending on whether those polypeptides also encode the prodomain. For example, where the prodomain is coupled to an antibody light chain, the relative quantity of clipped impurity (i.e., % clipped impurity) is calculated by dividing the % Peak Area of the light chain clipped impurity in the eluate by the sum of: a) the % Peak Area of the light chain clipped impurity in the eluate and b) the % PeakArea of the intact light chain in the eluate. Peak areas for clipped prodomain-encoding polypeptide and intact prodomain-encoding polypeptide are readily determined by a reducing SDS-cGE assay, such as the assay described in Example 1.

Thus, in these embodiments, the ratio of % clipped impurity in the aqueous feedstock to % clipped impurity in the eluate is, correspondingly, at least about 2, or at least about 3, or at least about 4, or at least about 5, or at least about 6, or at least about 7, or at least about 8, or at least about 9, or at least about 10, or at least about 15, or at least about 20, as measured by reducing SDS-capillary gel electrophoresis (SDS-cGE) assay, such as the assay described in Example 1.

In some embodiments, the relative quantity of clipped impurity in the aqueous feed compared to the relative quantity of clipped impurity in the eluate corresponds to a level of reduction that is at least about 50%, or at least about 55%, or at least about 60%, or at least about 65%, or at least about 70%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%, as determined by reducing SDS-cGE, such as the assay described in Example 1. Percent (%) reduction of clipped impurity is determined by the following formula:

$$\% reduction of clipped impurity = \frac{{( \% clipped impurity )}_{aqueous feedstock} - {( \% clipped impurity )}_{eluate}}{{( \% clipped impurity )}_{Aqueous Feedstock}} \times 100 ,$$

where % clipped impurity for each of the aqueous feedstock and eluate are as defined above.

In some embodiments, the eluate comprises less than about 50%, or less than about 45%, or less than about 40%, or less than about 35%, or less than about 30%, or less than about 25%, or less than about 24%, or less than about 23%, or less than about 22%, or less than about 21%, or less than about 20%, or less than about 19%, or less than about 18%, or less than about 17%, or less than about 16%, or less than about 15%, or less than about 14%, or less than about 13%, or less than about 12%, or less than about 11%, or less than about 10% of the relative quantity of clipped impurity present in the aqueous feedstock. Relative quantity of clipped impurity in the eluate as a percent of clipped impurity in the aqueous feedstock is determined as follows:

$$\frac{{( \% clipped impurity )}_{eluate}}{{( \% clipped impurity )}_{Aqueous Feedstock}} \times 100 ,$$

where % clipped impurity for each of the aqueous feedstock and eluate are as defined above.

In some embodiments, the eluate comprises less than about 15%, or less than about 14%, or less than about 13%, or less than about 12%, or less than about 11%, or less than about 10%, or less than about 10%, or less than about 9%, or less than about 8%, or less than about 7%, or less than about 6% of the relative quantity of quantity of clipped impurity present in the aqueous feedstock, as measured by reducing SDS-cGE. In some embodiments, the eluate comprises a range of about 2% to 15%, or about 3% to about 15%, or about 4% to about 15%, or about 5% to about 15%, or about 2% to about 10%, or about 3% to about 10% of the relative quantity of quantity of clipped impurity present in the aqueous feedstock, as measured by reducing SDS-cGE.

The hydrophobic process is thus effective at generating an eluate (and corresponding purified composition of intact activatable antibody) having relatively low levels of clipped impurity. In certain embodiments, the eluate (and corresponding purified composition of intact activatable antibody) comprises a relative quantity of clipped impurity in the range of from about 0.1% to about 15% clipped impurity, or from about 0.1% to about 10% clipped impurity, or from about 0.1% to about 10% clipped impurity, or from about 0.1% to about 5% clipped impurity, or from about 0.1% to about 4% clipped impurity, or from about 0.1% to about 3% clipped impurity, or from about 0.1% to about 2% clipped impurity, or from about 0.1% to about 1% clipped impurity, as determined by reducing SDS-cGE. Percent clipped impurity is calculated as defined above. Percent clipped impurity may be measured in a sample taken from the eluate immediately after a separation procedure is completed, and may be either analyzed by SDS-cGE immediately or frozen until analyzed by SDS-cGE.

In some embodiments, the eluate (and corresponding purified composition of intact activatable antibody) comprises less than about 5%, or less than about 4%, or less than about 3%, or less than about 2%, or less than about 1%, or less than about 0.9%, or less than about 0.8%, or less than about 0.7%, or less than about 0.6%, or less than about 0.5% clipped impurity, as measured by reducing SDS-cGE. In other embodiments, the eluate comprises less than about 2%, or less than about 1%, or less than about 0.9%, or less than about 0.8%, or less than about 0.7%, or less than about 0.6%, or less than about 0.5% clipped impurity, as measured by reducing SDS-cGE. In certain embodiments, the eluate (and corresponding purified composition of intact activatable antibody) comprises no detectable clipped impurity.

In some embodiments, the aqueous feedstock comprises one or more further impurity selected from the group consisting of host cell protein (HCP), high molecular weight species (HMWS), and a combination thereof. The processes of the present invention are effective at substantially reducing the quantities of these impurities as well. HCP and HMWS both appear to be largely retained in the column along with clipped impurity during the elution step.

In certain embodiments, the further impurity is HCP. In these embodiments, the process is highly effective at removing HCP from the process stream. In some embodiments, the level of reduction effected by the hydrophobic chromatography process is at least about 3-fold, or at least about 4-fold-, or at least about 5-fold, or at least about 6-fold, or at least about 7-fold, or at least about 8-fold, or at least about 9-fold, or at least about 10-fold, on a parts per million (ppm) basis, as determined by a corresponding HCP ELISA assay. Fold-reduction in HCP is determined by dividing the quantity of HCP in the aqueous feed by the quantity of HCP in the eluate on a ppm basis, as determined by a corresponding HCP ELISA assay, i.e.,

$$\frac{{( HCP in ppm )}_{Aqueous Feedstock}}{{( HCP in ppm )}_{Eluate}} .$$

The quantity of HCP can be readily determined using Host Cell Protein ELISA kits that are commercially available for a number of different host cells, including, for example, mammalian, yeast, bacterial, and transgenic host species. The term “corresponding HCP ELISA assay” is used herein to refer to a host cell protein ELISA assay that employs antibodies to proteins associated with the host cell used to produce the activatable antibody. Correspondingly, in some embodiments, the ratio of quantity of HCP in the aqueous feedstock to the quantity of HCP in the eluate is at least about 4, or at least about 5, or at least about 6, or at least about 7, or at least about 8, or at least about 9, or at least about 10, on a parts per million (ppm) basis, as determined by a corresponding HCP ELISA assay.

In terms of percent (%) reduction in HCP, in certain embodiments, the level of reduction is at least about 50%, or is at least about 55%, or is at least about 60%, or is at least about 65%, or is at least about 70%, or is at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, on a ppm basis, as determined by a corresponding HCP ELISA assay. Percent reduction of HCP is determined by the formula:

$$\frac{[ {( ppm HCP )}_{aqueous feedstock} - {( ppm HCP )}_{eluate} ]}{{( ppm HCP )}_{aqueous feedstock}} \times 100.$$

Correspondingly, in some embodiments, the eluate comprises less than about 50%, or less than about 45%, or less than about 40%, or less than about 35%, or less than about 25%, or less than about 24%, or less than about 23%, or less than about 22%, or less than about 21%, or less than about 20%, or less than about 19%, or less than about 18%, or less than about 17%, or less than about 16%, or less than about 15%, or less than about 14%, or less than about 13%, or less than about 12%, or less than about 11%, or less than about 10% of the HCP present in the aqueous feedstock. The relative quantity of HCP in the eluate as a percent of HCP present in the aqueous feedstock is determined by the formula:

$$\frac{{( ppm HCP )}_{eluate}}{{( ppm HCP )}_{aqueous feedstock}} \times 100.$$

In some embodiments, the eluate (and corresponding purified composition of intact activatable antibody) comprises less than about 150 ppm, or less than about 140 ppm, or less than about 130 ppm, or less than about 120 ppm, or less than about 110 ppm, or less than about 100 ppm, or less than about 90 ppm, or less than about 80 ppm, or less than about 70 ppm, or less than about 60 ppm, or less than about 50 ppm, or less than about 45 ppm, or less than about 40 ppm, or less than about 35 ppm, or less than about 30 ppm, or less than about 25 ppm, or less than about 20 ppm, or less than about 15 ppm, or less than about 10 ppm HCP, as measured by a corresponding HCP ELISA. In certain embodiments, the eluate (and corresponding purified composition of intact activatable antibody) comprises no detectable HCP.

In other embodiments, the eluate (and corresponding composition of purified activatable antibody) comprises a quantity of HCP in the range of from about 0.5 ppm HCP to about 150 ppm HCP, or from about 0.5 ppm HCP to about 140 ppm HCP, or 0.5 ppm HCP to about 130 ppm HCP, or from about 0.5 ppm HCP to about 120 ppm, or from about 0.5 ppm HCP to about 110 ppm, or from about 0.5 ppm HCP to about 100 ppm HCP, or from about 0.5 ppm HCP to about 90 ppm HCP, or from about 0.5 ppm HCP to about 90 ppm HCP, or from about 0.5 ppm HCP to about 80 ppm HCP, or from about 0.5 ppm HCP to about 70 ppm HCP, or from about 0.5 ppm HCP to about 60 ppm HCP, or from about 0.5 ppm to about 50 ppm HCP, or from about 0.5 ppm HCP to about 45 ppm HCP, or from about 0.5 ppm HCP to about 40 ppm HCP, or from about 0.5 ppm HCP to about 40 ppm HCP, or from about 0.5 ppm HCP to about 35 ppm HCP, or from about 0.5 ppm HCP to about 30 ppm HCP, or from about 1 ppm HCP to about 25 ppm HCP, or from about 1 ppm HCP to about 20 ppm HCP, or from about 0.5 ppm HCP to about 15 ppm HCP, or from about 0.5 ppm HCP to about 10 ppm HCP.

In certain embodiments, the eluate (and corresponding purified composition of activatable antibody) comprises a quantity of HCP in the range of from about 1 ppm HCP to about 150 ppm HCP, or from about 1 ppm HCP to about 140 ppm HCP, or 1 ppm HCP to about 130 ppm HCP, or from about 1 ppm HCP to about 120 ppm, or from about 1 ppm HCP to about 110 ppm, or from about 1 ppm HCP to about 100 ppm HCP, or from about 1 ppm HCP to about 90 ppm HCP, or from about 1 ppm HCP to about 90 ppm HCP, or from about 1 ppm HCP to about 80 ppm HCP, or from about 1 ppm HCP to about 70 ppm HCP, or from about 1 ppm HCP to about 60 ppm HCP, or from about 1 ppm to about 50 ppm HCP, or from about 1 ppm HCP to about 45 ppm HCP, or from about 1 ppm HCP to about 40 ppm HCP, or from about 1 ppm HCP to about 40 ppm HCP, or from about 1 ppm HCP to about 35 ppm HCP, or from about 1 ppm HCP to about 30 ppm HCP, or from about 1 ppm HCP to about 25 ppm HCP, or from about 1 ppm HCP to about 20 ppm HCP, or from about 1 ppm HCP to about 15 ppm HCP, or from about 1 ppm HCP to about 10 ppm HCP.

In other embodiments, the further impurity is HMWS, either alone or in combination with HCP. HMWS can be detected and quantified by size exclusion (SE)-HPLC. An illustrative SE-HPLC assay is described in Example 1. High molecular weight species (HMWS) is detected to the left side of the main peak on the chromatogram. In these embodiments, the hydrophobic chromatography process is highly effective at removing HMWS from the process stream. In some embodiments, the level of reduction in quantity of HMWS effected by the hydrophobic chromatography process is at least about 2-fold, or at least about 3-fold, or at least about 4-fold, or at least about 5-fold, where HMWS is quantified by size exclusion (SE)-HPLC. An exemplary SE-HPLC assay is described in Example 1. Fold reduction of HMWS, and correspondingly, ratio of quantity of HMWS in the aqueous feedstock to quantity of HMWS in the eluate, are both determined by the formula:

$$\frac{{( \% Peak area HMWS )}_{aqueous feedstock}}{{( \% Peak area HMWS )}_{eluate}}$$

where:

(% Peak area HMWS) aqueous feedstock is the sum of all peaks corresponding to HMWS in the aqueous feedstock divided by the total of all peak area (i.e., total peak area), as determined by SE-HPLC assay; and
(% Peak area HMWS) eluate is the sum of all peaks corresponding to HMWS in the eluate divided by the total of all peak area (total peak area), as determined by SE-HPLC.

Thus, in these embodiments, the ratio of quantity of HMWS in the aqueous feedstock to the quantity of HMWS in the eluate is, correspondingly, at least about 2, or at least about 3, or at least about 4, or at least about 5, where quantity of HMWS is determined by SE-HPLC.

In some embodiments, the level of reduction of HMWS effected by the hydrophobic chromatography process in terms of percent (%) reduction of HMWS is at least about 50%, or is at least about 55%, or is at least about 60%, or is at least about 65%, or is at least about 70%, or is at least about 75%, or at least about 76%, or is at least about 77%, or is at least about 78%, or is at least about 79%, or is at least about 80%, or is at least about 81%, or is at least about 82%, or is at least about 83%, or is at least about 84%, or is at least about 85%, or is at least about 86%, or is at least about 87%, or is at least about 88%, or is at least about 89%, or is at least about 90%, or is at least about 91%, or is at least about 92%, or is at least about 93%, or is at least about 94%, or is at least about 95%, or is at least about 96%, or is at least about 98%, or is at least about 99%, as determined by SE-HPLC. Percent reduction of HMWS is determined according to the following formula:

$$\frac{\begin{matrix}[ {( \% Peak Area HMWS )}_{aqueous feedstock} - \\ {( \% Peak Area HMWS )}_{eluate} ]\end{matrix}}{{( \% Peak Area HMWS )}_{aqueous feedstock}} \times 100$$

where % Peak Area HMWS for the aqueous feedstock and eluate are defined hereinabove.

In some embodiments, the relative quantity of HMWS in the eluate as a percent of HMWS present in the aqueous feedstock is less than about 50%, or less than about 45%, or less than about 40%, or less than about 35%, or less than about 25%, or less than about 30%, or less than about 25%, or less than about 20%, or less than about 15%, or less than about 15% of the HMWS present in the aqueous feedstock, as determined by SE-HPLC. The relative quantity of HMWS in the eluate as a percent of HMWS present in the aqueous feedstock is computed as:

$$\frac{{( \% Peak Area HMWS )}_{eluate}}{{( \% Peak Area HMWS )}_{aqueous feedstock}} \times 100$$

where % Peak Area HMWS for the aqueous feedstock and eluate are defined hereinabove.

In some embodiments, the eluate (and corresponding purified composition of intact activatable antibody) comprises less than about 5%, or less than about 4%, or less than about 3%, or less than about 2%, or less than about 1% HMWS, as determined by SE-HPLC, where % HMWS corresponds to (% Peak Area HMWS) eluate and is determined as described above.

In other embodiments, the eluate (and corresponding purified composition of intact activatable antibody) comprises less than about 3%, or less than about 2%, or less than about 1% HMWS. In some embodiments, the eluate comprises less than about 2%, or less than about 1% HMWS.

In some embodiments, the eluate (and corresponding purified composition of intact activatable antibody) comprises a quantity of HMWS in the range of from about 0.1%, 0.2%, or 0.3% HMWS to about 5% HMWS, or from about 0.2% HMWS to about 4% HMWS, or from about 0.2% HMWS to about 3% HMWS, or from about 0.2% HMWS to about 2% HMWS, or from about 0.2% HMWS to about 1% HMWS, as determined by SE-HPLC, where % is determined on the basis of total peak area. In certain embodiments, the eluate (and corresponding purified composition of intact activatable antibody) comprises no detectable HMWS.

In some embodiments of the processes described herein, the aqueous feedstock comprises greater than about 0.5% clipped impurity, or greater than about 0.6%, or greater than about 0.7%, or greater than about 0.8%, or greater than about 0.9%, or greater than about 1%, or greater than about 1.5%, or greater than about 2%, or greater than about 2.5%, or greater than about 3%, or greater than about 3.5%, or greater than about 4%, or greater than about 4.5%, or greater than about 5%, or greater than about 5.5%, or greater than about 6%, or greater than about 6.5%, or greater than about 7%, or greater than about 7.5%, or greater than about 8%, or greater than about 8.5%, or greater than about 9%, or greater than about 9.5%, or greater than about 10%, or greater than about 10.5%, or greater than about 11%, or greater than about 11.5%, or greater than about 12%, or greater than about 12.5%, or greater than about 13%, or greater than about 13.5% clipped impurity, as measured by reducing SDS-cGE. Percent clipped impurity is defined hereinabove.

The processes of the present invention generate an eluate that is a highly pure composition of intact activatable antibody. The resulting eluate (and corresponding purified composition of intact activatable antibody) often comprises at least about 90%, or at least about 91%, or at least about 92%, or at least about 93%, or at least about 94%, or at least about 95%, or at least about 96%, or at least about 97%, or at least about 98%, or at least about 98.5%, or at least about 99% intact activatable antibody, as measured by reducing SDS-cGE. An illustrative SDS-cGE assay is described in Example 1. Percent intact impurity is calculated as described hereinabove (i.e., (% intact activatable antibody) eluate).

In one aspect, the present disclosure includes a process for producing a composition including: (A) greater than 95% intact activatable antibody comprising a MM, a CM, and a AB; and (B) 0.05 to 5% clipped variant thereof, the process including loading an aqueous feedstock comprising water, (A), (B), and a first salt onto a chromatography column, wherein the chromatography column comprises a stationary phase that comprises a support matrix and hydrophobic ligands bound thereto, and eluting the chromatography column with an eluent comprising water and a second salt to obtain the composition. In one aspect, the process includes reducing the amount of clipped variant in the process stream by 75 to 90%. In one aspect, the process includes reducing the amount of HCP in the process stream by 75 to 90%. In one aspect, the process includes reducing the amount of HMWS in the process stream by 75 to 90%. In one aspect, the process includes reducing the amount of clipped variant, HCP, and HMWS in the process stream by 70 to 95%.

Activatable antibody is typically produced biologically by culturing cells engineered to express the desired intact activatable antibody. For example, the cells may be mammalian host cells. In some aspects, the cells may be human embryonic kidney (HEK) cells, e.g., HEK293 cells, or Chinese hamster ovary (CHO) cells. In these processes, activatable antibody is harvested from the cell culture as a cell culture supernatant, cellular lysate, or other like composition derived from the cell culture that contains the activatable antibody. A bioharvest composition is obtained by separating the cells and cellular debris from the supernatant or lysate, using, for example, centrifugation, filtration, or other solid-liquid separation process.

The hydrophobic chromatography process is often deployed downstream of the cell culturing step, with, optionally, one or more intervening unit operations for removing at least a portion of non-immunoglobulin proteins, host cell proteins, and other impurities that may be present in the bioharvest composition, and if applicable, any bulk intermediate product composition.

Each cell culturing step, the optional one or more intervening unit operations, and hydrophobic chromatography process may be conducted as a batch-wise process, or optionally, any two or more of the foregoing unit operations may be conducted as a continuous or semi-continuous process in an optionally integrated system. In some embodiments, the cell culturing step is performed as a fed-batch operation. In some embodiments, the cell culturing step is performed as a continuous feed or perfusion operation.

In some embodiments, the bioharvest composition and/or one or more bulk intermediate product compositions is/are staged over a period of time prior to being fed to the next unit operation. Bulk intermediate product compositions may be optionally conditioned with the addition of one or more conditioning agents to render it suitable as feed for the next, subsequent unit operation. Exemplary conditioning agents include for example, a buffer (i.e., one or more buffering agents), a salt (e.g., the first salt), a base, an acid, and the like.

Thus, aqueous feedstocks employed in the practice of the present invention may comprise a bioharvest composition or a bulk intermediate product composition. In some embodiments, prior to step (a), the process comprises:

(a⁰) subjecting a bioharvest composition comprising the intact activatable antibody activatable antibody and the corresponding clipped impurity to one or more intervening unit operations selected from the group consisting of a centrifugation step, a filtration step, an affinity chromatography step, a virus inactivation step, a size exclusion chromatography step, a virus filtration step, and an ion exchange (IEX) chromatography step to produce one or more bulk intermediate product compositions, wherein the aqueous feedstock comprises at least one bulk intermediate product composition. In some embodiments, the process comprises a combination of at least two or more intervening unit operations.

In some embodiments, the bioharvest composition is subjected to at least two or more, or in certain embodiments, at least three or more serial intervening unit operations selected from the group consisting of a centrifugation step, a filtration step, an affinity chromatography step, a virus inactivation step, a size exclusion chromatography step, a virus filtration step, and an ion exchange chromatography step. In some embodiments, the bioharvest composition is subjected to an affinity chromatography step, and often, additionally a virus inactivation step, and an optional ion exchange chromatography step. For example, affinity chromatography may be employed to separate immunoglobulin (Ig)-containing components from other components in the bioharvest composition by using an immobilized ligand such as, for example, Protein A (i.e., a “Protein A chromatography” step), Protein G (i.e., a “Protein G chromatography” step), and the like. In certain embodiments, the one or more intervening unit operations of step (a⁰) comprises an affinity chromatography step and an ion exchange chromatography step. In some embodiments, the one or more intervening unit operations of step (a⁰) comprises an affinity chromatography step, a virus inactivation step, a filtration step (e.g., a tangential flow filtration step, an ultrafiltration step, a diafiltration step, and the like), and an ion exchange chromatography step. In certain embodiments, the ion exchange chromatography step is an anion exchange chromatography step. In some aspects, the method of the present disclosure includes methods that exclude any one or combination of the operations described in this disclosure. For example, a method of the present disclosure may exclude an anion exchange step. As another example, a method of the present disclosure may exclude a cation exchange step. As another example, a method of the present disclosure may exclude a size exclusion chromatography step.

When the process employs one or more intervening unit operations, the bulk intermediate product composition produced by the intervening unit operation immediately upstream of the hydrophobic chromatography process is referred to herein as the “pre-feed”. In some embodiments, the pre-feed may be used directly as the aqueous feedstock. In certain embodiments, the pre-feed may be conditioned by adding one or more components, such as, for example, the first salt, one or more buffering agents (such as, for example, a salt, an acid, and/or a base), and the like.

The eluate (and corresponding purified composition of intact activatable antibody), enriched with intact activatable antibody relative to clipped impurity, may be optionally subjected to one or more downstream unit operations to generate a downstream product composition. Illustrative downstream unit operations include, for example, a (further) purification process, a chemical synthesis process, a dilution process, a solvent exchange process, a formulating process, a lyophilization process, or any combination of two or more thereof. For example, in one embodiment, the process further comprises subjecting the eluate to one or more downstream unit operations selected from the group consisting of a centrifugation step, a filtration step (e.g., tangential flow filtration, ultrafiltration, diafiltration, and the like), an affinity chromatography step, a virus inactivation step, a size exclusion chromatography step, a virus filtration step, an ion exchange chromatography step, and any combination of two or more thereof. The eluate and downstream product compositions often have either no detectable quantities, or very low quantities of clipped impurity, and/or HCP, and/or HMWS. The eluate and downstream product compositions may be substantially free of clipped impurity.

In some embodiments, the purified intact activatable antibody composition generated by the processes of the present invention (e.g., the eluate or a downstream product composition) is subjected to a chemical conjugation reaction by contacting the eluate or downstream product composition with a conjugation reagent under conditions sufficient to generate a conjugated activatable antibody. Conjugation moieties function to impart an additional property to the activatable antibody, such as, for example, extended half-life (e.g., where the conjugation moiety is a half-life extender, such as, for example, a polyethylene glycol (PEG) moiety, a human serum albumin (HSA moiety), and the like); cytotoxicity (where the conjugation moiety is all or part of a toxin, such as, for example, a dolastin or derivative thereof (e.g., auristatin E, AFP, MMAF, MMAE, MMAD, DMAF, DMAE, and the like, and derivatives thereof), a maytansinoid or derivative thereof, DM1, DM4, a duomycin or derivative thereof, a calicheamicin or derivative thereof, a pyrrolobenzodiazepine or derivative or dimer thereof, a heavy metal (e.g., barium, gold, platinum, and the like), a pseudomonas toxin A variant (e.g., PE38, ZZ-PE38, and the like), ZJ-101, OSW-1, a 4-nitrobenzyloxycarbonyl derivative of O6-benzylguanine, a toposiomerase inhibitor, hemiasterlin, cephalotaxine, homoharringonine, a pyrrolobenzodiazepine dimer, a pyrrolobenzodiazepene, a functionalized pyrrolobenzodiazepene, a functionalized pyrrolbenzodiazepene dimer, a calicheamicin, a podophyllotoxin, a taxane, a vinca alkaloid, and the like, as well as any of a variety of other known cytotoxic agents); anti-viral activity (e.g., where the conjugation moiety is all or a portion of Acyclovir, Vira A, Symetrel, Turbostatin, a Phenstatin, Hydroxyphenstatin, Spongistatin 5, Spongistatin 7, Halistatin 1, Halistatin 2, Halistatin 3, a modified bryostatin, a halocomstatin, pyrrolobenzimadazole, cibrostatin6, doxaliform, an anthracycline analogue, a cemadotin analogue (e.g., CemCH2-SH), and the like); antifungal activity (e.g., wherein the conjugation moiety is all or a portion of Nystatin, and the like); anti-neoplastic activity (e.g., wherein the conjugation moiety is all or a portion of Adriamycin, cerubidine, bleomycin, alkeran, velban, oncovin, fluorouracil, methotrexate, thiotepa, bisantrene, novantrone, thioguanine, procarabizine, cytarabine, and the like); anti-bacterial activity (e.g., wherein the conjugation moiety is all or a portion of an aminoglycoside, streptomycin, neomycin, kanamycin, amikacin, gentamicin, tobramycin, Streptomycin B, spectinomycin, ampicillin, sulfanilamide, polymyxin, chloramphenicol, and the like); anti-mycoplasmal activity (e.g., where the conjugation moiety is all or a portion of tylosine, spectinomycin, and the like); as well as any of a variety of other desirable additional properties.

Conjugation moieties that impart such desired properties and functions can be readily conjugated to the activatable antibody using methods and reactive linkers that are known in the art. In some embodiments, compositions of conjugated activatable antibody prepared from purified activatable antibody compositions as described herein, are also substantially free of clipped impurity and/or HCP and/or HMWS. With respect to clipped impurity, HCP, and HMWS, these conjugated activatable antibody compositions often have the same purity and impurity profiles as the eluate and purified intact activatable antibody compositions described herein.

In a further embodiment, the present invention provides purified intact activatable antibody compositions that can be produced by the processes of the present invention. In some embodiments, the purified intact activatable antibody composition comprises at least about 90% intact activatable antibody, as measured by reducing SDS-cGE, wherein % intact activatable antibody is defined hereinabove; less than about 15% clipped impurity, as measured by reducing SDS-cGE, wherein % clipped impurity is defined hereinabove; less than about 5% HMWS, as measured by SE-HPLC, wherein % HMWS is defined hereinabove; and less than about 150 ppm HCP, as measured by a corresponding HCP ELISA assay. In certain of these embodiments, the purified intact activatable antibody composition comprise at least about 91%, or at least about 92%, or at least about 93%, or at least about 94%, or at least about 95%, or at least about 96%, or at least about 97%, or at least about 98%, or at least about 99% intact activatable antibody.

In some of these purified intact activatable antibody compositions, the composition comprises less than about 14%, or less than about 13%, or less than about 12%, or less than about 11%, or less than about 10%, or less than about 9%, or less than about 8%, or less than about 7%, or less than about 6% clipped impurity, as measured by SDS-cGE. In certain embodiments, the purified intact activatable antibody composition comprises less than about 5%, or less than about 4%, or less than about 3%, or less than about 2%, or less than about 1%, or less than about 0.9%, or less than about 0.8%, or less than about 0.7%, or less than about 0.6%, or less than about 0.5% clipped impurity, as measured by a reducing SDS-cGE assay. In other embodiments, the purified intact activatable antibody composition comprises less than about 2%, or less than about 1%, or less than about 0.8%, or less than about 0.8%, or less than about 0.7%, or less than about 0.6%, or less than about 0.5% clipped impurity, as measured by SDS-cGE. An illustrative SDS-cGE assay is described in Example 1. In certain embodiments, the purified intact activatable antibody composition comprises no detectable clipped impurity, as measured by reducing SDS-cGE.

In certain embodiments, the purified intact activatable antibody composition comprises a relative quantity of clipped impurity in the range of from about 0.1% to about 15% clipped impurity, or from about 0.1% to about 10% clipped impurity, or from about 0.1% to about 10% clipped impurity, or from about 0.1% to about 5% clipped impurity, or from about 0.1% to about 4% clipped impurity, or from about 0.1% to about 3% clipped impurity, or from about 0.1% to about 2% clipped impurity, or from about 0.1% to about 1% clipped impurity, as determined by reducing SDS-cGE. In certain embodiments, the purified intact activatable antibody composition comprises no detectable clipped impurity, as determined by reducing SDS-cGE.

In some embodiments, the purified intact activatable antibody composition comprises less than about 140 ppm HCP, or less than about 130 ppm HCP, or less than about 120 ppm HCP, or less than about 110 ppm HCP, or less than about 100 ppm HCP, or less than about 90 ppm HCP, or less than about 80 ppm HCP, or less than about 70 ppm HCP, or less than about 60 ppm HCP, or less than about 50 ppm HCP, or less than about 45 ppm HCP, or less than about 40 ppm HCP, or less than about 35 ppm HCP, or less than about 30 ppm HCP, or less than about 25 ppm HCP, or less than about 20 ppm HCP, or less than about 15 ppm HCP, or less than about 10 ppm HCP, as measured by a corresponding HCP ELISA. In some of these embodiments, the purified activatable antibody composition comprises less than about 50 ppm, or less than about 45 ppm, or less than about 40 ppm, or less than about 35 ppm, or less than about 30 ppm, or less than about 25 ppm, or less than about 20 ppm, or less than about 15 ppm, or less than about 10 ppm HCP, as measured by a corresponding HCP ELISA assay. In certain embodiments, the purified intact activatable antibody composition comprises no detectable HCP, as measured by a corresponding host cell ELISA.

In other embodiments, the purified intact activatable antibody composition comprises a quantity of HCP in the range of from about 0.5 ppm HCP to about 150 ppm HCP, or from about 0.5 ppm HCP to about 140 ppm HCP, or 0.5 ppm HCP to about 130 ppm HCP, or from about 0.5 ppm HCP to about 120 ppm, or from about 0.5 ppm HCP to about 110 ppm, or from about 0.5 ppm HCP to about 100 ppm HCP, or from about 0.5 ppm HCP to about 90 ppm HCP, or from about 0.5 ppm HCP to about 90 ppm HCP, or from about 0.5 ppm HCP to about 80 ppm HCP, or from about 0.5 ppm HCP to about 70 ppm HCP, or from about 0.5 ppm HCP to about 60 ppm HCP, or from about 0.5 ppm to about 50 ppm HCP, or from about 0.5 ppm HCP to about 45 ppm HCP, or from about 0.5 ppm HCP to about 40 ppm HCP, or from about 0.5 ppm HCP to about 40 ppm HCP, or from about 0.5 ppm HCP to about 35 ppm HCP, or from about 0.5 ppm HCP to about 30 ppm HCP, or from about 1 ppm HCP to about 25 ppm HCP, or from about 1 ppm HCP to about 20 ppm HCP, or from about 0.5 ppm HCP to about 15 ppm HCP, or from about 0.5 ppm HCP to about 10 ppm HCP.

In certain embodiments, the purified intact activatable antibody composition comprises a quantity of HCP in the range of from about 1 ppm HCP to about 150 ppm HCP, or from about 1 ppm HCP to about 140 ppm HCP, or 1 ppm HCP to about 130 ppm HCP, or from about 1 ppm HCP to about 120 ppm, or from about 1 ppm HCP to about 110 ppm, or from about 1 ppm HCP to about 100 ppm HCP, or from about 1 ppm HCP to about 90 ppm HCP, or from about 1 ppm HCP to about 90 ppm HCP, or from about 1 ppm HCP to about 80 ppm HCP, or from about 1 ppm HCP to about 70 ppm HCP, or from about 1 ppm HCP to about 60 ppm HCP, or from about 1 ppm to about 50 ppm HCP, or from about 1 ppm HCP to about 45 ppm HCP, or from about 1 ppm HCP to about 40 ppm HCP, or from about 1 ppm HCP to about 40 ppm HCP, or from about 1 ppm HCP to about 35 ppm HCP, or from about 1 ppm HCP to about 30 ppm HCP, or from about 1 ppm HCP to about 25 ppm HCP, or from about 1 ppm HCP to about 20 ppm HCP, or from about 1 ppm HCP to about 15 ppm HCP, or from about 1 ppm HCP to about 10 ppm HCP. In some embodiments, the purified intact activatable antibody composition comprises less than about 4%, or less than about 3%, or less than about 2%, or less than about 1% HMWS, as determined by SE-HPLC, where % HMWS is defined hereinabove.

In certain embodiments, the purified intact activatable antibody composition comprises a quantity of HMWS in the range of from about 0.2% HMWS to about 5% HMWS, or from about 0.2% HMWS to about 4% HMWS, or from about 0.2% HMWS to about 3% HMWS, or from about 0.2% HMWS to about 2% HMWS, or from about 0.2% HMWS to about 1% HMWS, as determined by SE-HPLC. In some embodiments, the purified intact activatable antibody composition comprises no detectable HMWS.

In some aspects, the purified intact activatable antibody composition includes greater than 90% intact activatable antibody and 0.05 to 5% clipped variant, as measured by SDS-cGE. In some aspects, the composition includes greater than 90% intact activatable antibody and 0.05 to 5% clipped variant (as determined by SDS-cGE), less than 150 ppm host cell proteins (HCP) (as determined by host cell ELISA), and less than 5% high molecular weight species (HMWS) (as determined by SE-HPLC). In some aspects, the composition includes greater than 96% intact activatable antibody, 0.05 to 4% clipped variant, less than 150 ppm host cell proteins (HCP) and less than 5% HMWS. In some aspects, the composition includes greater than 97% intact activatable antibody, 0.05 to 3% clipped variant, less than 150 ppm host cell proteins (HCP) and less than 5% HMWS. In some aspects, the composition includes greater than 98% intact activatable antibody, 0.05 to 2% clipped variant, less than 150 ppm host cell proteins (HCP) and less than 5% HMWS. In some aspects, the composition includes greater than 95% intact activatable antibody, 0.05 to 5% clipped variant, less than 100 ppm host cell proteins (HCP) and less than 3% HMWS. In some aspects, the composition includes greater than 95% intact activatable antibody, 0.05 to 3% clipped variant, less than 100 ppm host cell proteins (HCP) and less than 3% HMWS. In some aspects, the composition includes greater than 95% intact activatable antibody, 0.05 to 2% clipped variant, less than 100 ppm host cell proteins (HCP) and less than 3% HMWS. In some aspects, the composition includes greater than 95% intact activatable antibody, 0.05 to 5% clipped variant, less than 100 ppm host cell proteins (HCP) and less than 2% HMWS. In some aspects, the composition includes greater than 95% intact activatable antibody, 0.05 to 5% clipped variant, less than 100 ppm host cell proteins (HCP) and less than 1.5% HMWS. In some aspects, the composition includes greater than 95% intact activatable antibody, 0.05 to 3% clipped variant, less than 100 ppm host cell proteins (HCP) and less than 1.5% HMWS. In some aspects, the composition includes greater than 95% intact activatable antibody, 0.05 to 2% clipped variant, less than 100 ppm host cell proteins (HCP) and less than 1.5% HMWS. In some aspects, the composition includes greater than 95% intact activatable antibody, 0.05 to 5% clipped variant, less than 25 ppm host cell proteins (HCP) and less than 1.5% HMWS. In some aspects, the composition includes greater than 95% intact activatable antibody, 0.05 to 3% clipped variant, less than 25 ppm host cell proteins (HCP) and less than 1.5% HMWS. In some aspects, the composition includes greater than 95% intact activatable antibody, 0.05 to 2% clipped variant, less than 25 ppm host cell proteins (HCP) and less than 1.5% HMWS. In some aspects, the composition includes greater than 95% intact activatable antibody, 0.05 to 2% clipped variant, less than 10 ppm host cell proteins (HCP) and less than 1.5% HMWS. In some aspects, the composition includes greater than 95% intact activatable antibody, 0.05 to 5% clipped variant, less than 25 ppm host cell proteins (HCP) and less than 1.0% HMWS. In some aspects, the composition includes greater than 95% intact activatable antibody, 0.05 to 3% clipped variant, less than 25 ppm host cell proteins (HCP) and less than 1.0% HMWS. In some aspects, the composition includes greater than 95% intact activatable antibody, 0.05 to 2% clipped variant, less than 25 ppm host cell proteins (HCP) and less than 1.0% HMWS. In some aspects, the composition includes greater than 95% intact activatable antibody, 0.05 to 2% clipped variant, less than 10 ppm host cell proteins (HCP) and less than 1.0% HMWS. In some aspects, the clipped variant is a single-arm clipped species.

The present invention also provides a pharmaceutical composition comprising the purified composition of intact activatable antibody of the present invention and one or more pharmaceutically acceptable components. In some embodiments, the pharmaceutically acceptable component is a pharmaceutically acceptable excipient. Such compositions may be prepared by adding the pharmaceutically acceptable excipient to a purified composition produced by the process of the present invention, such as, for example, the purified composition of intact activatable antibody of the present invention, or further composition derived therefrom. Pharmaceutically acceptable excipients that are suitable for use in compositions of the present invention are well known in the art, and include, for example, sterile water, a surfactant (e.g., a nonionic surfactant, a cationic surfactant, and/or an anionic surfactant), a buffering agent (an acid, a base, and/or a salt), an alcohol, a diol, a polyol, a sugar (e.g., a monosaccharide, a disaccharide, and/or a polysaccharide), a hydrophilic polymer (e.g., a polyethylene glycol (PEG), a polyvinyl alcohol, a solubilizing agent (e.g., a cyclodextrin, and the like), and the like. The composition may be in either liquid form or it may be a solid form, e.g., particulate form. For example, the solid form may be prepared by lyophilizing a corresponding liquid composition to form a lyophilized pharmaceutical composition.

When the pharmaceutical composition is in liquid form, it typically further comprises water (e.g., sterile water). In some embodiments, the composition further comprises water and one or more components selected from the group consisting of a surfactant, a buffering agent, a sugar, and any combination of two or more thereof. When the pharmaceutical composition is in solid (e.g., lyophilized) form, it typically comprises one or more components selected from the group consisting of a sugar and a buffering agent. In some embodiments, the composition comprises a sugar.

The processes and compositions of the present invention may employ any of a wide variety of activatable antibodies. As illustrated in the examples hereinbelow, the process is shown to be capable of producing purified compositions of intact activatable antibody with significant reduction in clipped impurity, as well as HCP and HMWS. In each instance where aqueous feedstock comprising these activatable antibodies was purified using the processes of the present invention, an eluate that was enriched in intact activatable antibody and substantially depleted of impurities that included clipped impurity, HMWS, eluted first. This observation was consistent across a variety of activatable antibody amino acid sequences. Thus, it is believed that the processes of the present invention are applicable to any of a variety of crude activatable antibody compositions regardless of the specific amino acid sequences (e.g., MM, CM, AB) of the activatable antibody. Accordingly, compositions that may be purified using the processes of the present invention may comprise an activatable antibody having an AB component capable of specifically binding to any one of a number of biological targets known in the art (when not masked) by, for example, incorporating VL and VH CDR amino acid sequences from antibodies known to bind to the desired biological target, or alternatively, or identified by using any of a variety of known antibody discovery screening platforms known in the art.

Illustrative classes of biological targets include cell surface receptors and secreted binding proteins (e.g., growth factors, and the like), soluble enzymes, structural proteins (e.g., collagen, fibronectin, and the like), and the like. Suitable biological targets include, for example, 1-02-LFA-3, α4-integrin, α-V-integrin, α4B1-integrin, AGR2, Anti-Lewis-Y, Apelin J receptor, APRIL, B7-H4, BAFF, BTLA, C5 complement, C-242, CA9, CA19-9 (Lewis a), carbonic anhydrase 9, CD2, CD3, CD6, CD9, CD11a, CD19, CD20, CD22, CD25, CD28, CD30, CD33, CD40, CD40L, CD41, CD44, CD44v6, CD47, CD51, CD52, CD56, CD64, CD70, CD71, CD74, CD80, CD81, CD86, CD95, CD117, CD125, CD132 (IL-2RG), CD133, CD137, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK1, DLL4, DPP-4, DSG1, EGFR, EGFRviii, Endothelin B receptor (ETBR), ENPP3, EpCAM, EPHA2, ERBB3, F protein of RSV, FAP, FGF-2, FGF-8, FGFR1, FGFR2, FGFR3, FGFR4, Folate receptor, GAL3ST1, G-CSF, G-CSFR, GD2, GITR, GLUT1, GLUT4, GM-CSF, GM-CSFR, GP IIb/IIIa receptors, GP130, GPIIB/IIIA, GPNMB, GRP78, Her2/neu, HVEM, Hyaluronidase, ICOS, IFNα, IFNβHGF, hGH, hyaluronidase, ICOS, IFNα, IFNβ, IFNγ, IgE, IgE receptor (FceRI), IGF, IGF1R, IL1B, IL1R, IL2, IL11, IL12p40, IL-12R, IL-12RB1, IL13, IL13R, IL15, IL17, IL18, IL21, IL23, IL23R, IL27/IL27R (wsx1), IL29, IL-31R, IL31/IL31R, IL-2R, IL4, IL4-R, IL6, IL-6R, Insulin Receptor, Jagged Ligands, Jagged 1, Jagged 2, LAG-3, LIF-R, Lewis X, LIGHT, LRP4, LRRC26, MCSP, Mesothelin, MRP4, MUC1, Mucin-16 (MUC16, CA-125), Na/K ATPase, Neutrophil elastase, NGF, Nicastrin, Notch Receptors, Notch 1, Notch 2, Notch 3, Notch 4, NOV, OSM-R, OX-40, PAR2, PDGF-AA, PDGF-BB, PDGFRα, PDGFRβ, PD-1, PD-L1, PD-L2, Phosphatidylserine, P1GF, PSCA, PSMA, RAAG12, RAGE, SLC44A4, Sphingosine 1 Phosphate, STEAP1, STEAP2, TAG-72, TAPA1, TGFβ, TIGIT, TIM-3, TLR2, TLR6, TLR7, TLR8, TLR9, TMEM31, TNFα, TNFR, TNFRS12A, TRAIL-R1, TRAIL-R2, Transferrin, Transferrin receptor, TRK-A, TRK-B, uPAR, VAPI, VCAM-1, VEGF, VEGF-A, VEGF-B, VEGF-C, VEGF-D, VEGFR1, VEGFR2, VEGFR3, VISTA, WISP-1, WISP-2, WISP-3, and the like.

The AB is formed from (and thus the activatable antibody comprises) a VL and a VH, which are linked together either directly or indirectly, for example, via a covalent or non-covalent bond. In some embodiments, the VL and VH are linked together by one or more disulfide bonds (e.g., via one or more Cys-Cys disulfide bridges), by a peptide linker, by a synthetic linker, by a naturally occurring linker, and the like. Structurally, each AB may be independently in any of a wide variety of formats, such as, for example, a Fab, a F(ab′)₂, a monospecific Fab₂, a bispecific Fab₂, a trispecific Fab₃, an scFv, a bispecific diabody, a trispecific triabody, an scFv-Fc, a minibody, a bispecific T cell engager (e.g., a BiTE™), a dual-affinity re-targeting antibody (DART antibody), and the like. Suitable bispecific formats include any one of a number of bispecific antibody formats known in the art, including those described in Kontermann, et al., “Bispecific Antibodies”, Drug Discovery Today (2015)20 (7): 838-847, which is incorporated herein by reference in its entirety.

In some embodiments, the activatable antibodies comprise a first VL having a set of first VL CDRs (i.e., a first VL CDR1, a first VL CDR2, and a first VL CDR3) and a first VH having a set of first VH CDRs (i.e., a first VH CDR1, a first VH CDR2, and a first VH CDR3), where the VL and VH together form the corresponding AB. In some embodiments, the activatable antibody also has a second AB that has a specific binding affinity for a second biological target, where the second AB comprises a second VL, a second VH, and a second prodomain that comprises a second MM and a second CM, wherein the second AB is coupled to the second prodomain. In these embodiments, the second VL comprises a set of second VL CDRs (i.e., a second VL CDR1, a second VL CDR2, and a second VL CDR3) and a second VH having a set of second VH CDRs (i.e., a second VH CDR1, a second VH CDR2, and a second VH CDR3), where the VL and VH together form the corresponding second AB. Purified compositions of activatable antibodies having additional ABs (from third VL and third VH amino acid sequences, and so on) may be prepared using the process of the present invention, where each additional AB has a corresponding MM and CM coupled to it. Each set of VL and VH corresponding to an AB may be encoded by a single polypeptide (such as, for example, an scFv, and the like), or two polypeptides (such as an antibody light chain and an antibody heavy chain). VH and VL CDR sequences having binding specificity for a wide variety of biological targets are known in the art and may be incorporated into the activatable antibodies employed in the processes and compositions of the present invention.

In some embodiments when the activatable antibody comprises a second VL and a second VH, the amino acid sequence of each CDR in the set of first VL CDRs is identical to the amino acid sequence of the corresponding CDR in the set of second VL CDRs. In certain embodiments, the amino acid sequence of each CDR in the set of first VH CDRs is identical to the amino acid sequence of the corresponding CDR in the set of second VH CDRs. In these embodiments, the first AB and the second AB (i.e., when not masked) typically have binding specificity for the same biological target species.

In some embodiments, the amino acid sequence of at least one CDR in the set of first VL CDRs is not identical to the amino acid sequence of the corresponding CDR in the set of second VL CDRs and/or the amino acid sequence of at least one CDR in the set of first VH CDRs is not identical to the amino acid sequence of the corresponding CDR in the set of second VH CDRs. In these embodiments, the first biological target and the second biological target may be the same or different. For example, the first AB and the second AB may bind to different epitopes or bind to overlapping epitopes on the same biological target. In some of these embodiments, the first biological target and the second biological target are not the same (i.e., the activatable antibody is a “multispecific” activatable antibody, such as a bispecific activatable antibody, and the like). Often, in these embodiments, at least one of the first and second biological targets is a cell surface receptor or ligand associated, for example, with cancer, cell proliferation, or an inflammatory process. In some of the bispecific embodiments, the first biological target is a cluster of differentiation 3 (CD3) T cell co-receptor. Usually, at least one of the other of the first and second biological targets is an extracellular membrane-bound protein in which expression from the cell or presence of the cell is associated with a diseased state.

In some embodiments, the VL and VH domains of the activatable antibody reside within an antibody light and an antibody heavy chain, respectively, each having incorporated with them, at least one additional component. For example, the light chain may comprise an amino acid sequence that encodes a VL and at least one additional component selected from the group consisting of a prodomain, a linker, a light constant domain (λ or κ), and a combination of any two or more thereof and the heavy chain may comprise an amino acid sequence that encodes a VH, and at least one additional component selected from the group consisting of a prodomain, a linker, one or more heavy constant domains (i.e., a CHI, a CH₂, and/or a CH₃ domain) and/or a hinge region, and a combination of any two or more thereof, provided that at least one of the light chain and heavy chain comprises an amino acid sequence that encodes a prodomain. In some embodiments, the heavy chain comprises an amino acid sequence that encodes an Fc region that corresponds substantially to a human immunoglobulin (Ig) class selected from the group consisting of an IgA, an IgD, an IgG, an IgE, and an IgM.

Often, the Fc domain comprises a native human Fc domain. In some embodiments, the Fc domain is an engineered human Fc domain that has an amino acid sequence that differs from a native human Fc domain. Engineered Fc domains often exhibit altered effector function relative to the corresponding native Fc domain. Such functions include, for example, enhanced antibody-dependent cell-mediated cytotoxicity (ADCC), enhanced antibody-dependent cellular phagocytosis (ADCP), enhanced complement-dependent cytotoxicity (CDC), reduced effector function, increased half-life, increased FcγRIIb binding, increased FcγRIIa binding, and the like.

Additional examples of engineered human Fc domains are known to those skilled in the art. Examples of Ig heavy chain constant region amino acids in which mutations in at least one amino acid leads to reduced Fc function include, but are not limited to, mutations in amino acid 228, 233, 234, 235, 236, 237, 239, 252, 254, 256, 265, 270, 297, 318, 320, 322, 327, 329, 330, and 331 of the heavy constant region. Examples of combinations of mutated amino acids are also known in the art, such as, but not limited to a combination of mutations in amino acids 234, 235, and 331, such as L234F, L235E, and P331S or a combination of amino acids 318, 320, and 322, such as E318A, K320A, and K322A.

Further examples of engineered Fc domains include, F243L/R292P/Y300L/V305I/P396 IgG1; S239D/1332E IgG1; S239D/1332E/A330L IgG1; S298A/E333A/K334A; in one heavy chain, L234Y/L235Q/G236W/S239M/H268D/D270E/S298A IgG1, and in the opposing heavy chain, D270E/K326D, A330M/K334E IgG; G236A/S239D/1332E IgG1; K326W/E333S IgG1; S267E/H268F/S324T IgG1; E345R/E430G/S440Y IgG1; N297A or N297Q or N297G IgG1; L235E IgG1; L234A/L235A IgG1; F234A/L235A IgG4; H268Q/V309L/A330S/P331S IgG2; V234A/G237A/P238S/H268A/V309L/A330S/P331S IgG2; M252Y/S254T/T256E IgG1; M428L/N434S IgG1; S267E/L328F IgG1; N325S/L328F IgG1, and the like. In some embodiments, the engineered Fc domain comprises one or more substitutions selected from the group consisting of N297A IgG1, N297Q IgG1, and S228P IgG4. Amino acid residue numbering is based on the EU numbering system.

Activatable antibodies employed in the practice of the present invention may exist in a variety of structural configurations. Illustrative structural formulae for activatable antibodies are provided below. It should be noted that although MM and CM are indicated as distinct components in the formulae below, in all exemplary embodiments (including formulae) disclosed herein it is contemplated that the amino acid sequences of the MM and the CM could overlap, e.g., such that the CM is completely or partially contained within the MM.

Within an activatable antibody, the CM may be positioned between the MM and AB, either directly or indirectly via one or more linkers. Often, each of the MM, CM, and AB components of the activatable antibody are arranged in a structure selected from the group consisting of, from N-terminal to C-terminal:

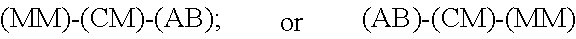

where MM, CM, and AB are as previously defined, and where each “-” refers independently to a direct or indirect (i.e., via a linker as described hereinbelow) linkage.

In many embodiments, the activatable antibody may comprise one or more linkers to impart flexibility at one or more of the MM-CM junction, the CM-AB junction, or both. For example, in certain embodiments, the activatable antibody may comprise MM, CM, and AB components arranged in a structure selected from the group consisting of, from either N- to C-terminal or C-terminal to N-terminal:

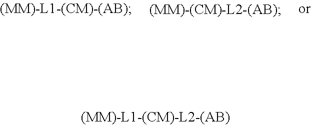

wherein MM, CM, and AB are as defined hereinabove; wherein each linker, L1 and L2, may be the same or different, and each independently may be optionally present or absent.

In some embodiments, the intact activatable antibody comprises a first AB, wherein the first VL is encoded by a first light chain and wherein the first VH is encoded by a first heavy chain, and wherein the first light chain further encodes one or more components selected from the group consisting of a prodomain, a linker, a light constant domain, and a combination of any two or more thereof, and wherein the first heavy chain further encodes one or more components selected from the group consisting of a prodomain, a linker, one or more heavy constant domains, a hinge region, and a combination of any two or more thereof, provided that only one of the light chain and the heavy chain encodes the prodomain. In certain embodiments, the intact activatable antibody further comprises an Fc domain, as described in more detail hereinabove. In some of these embodiments, the activatable antibody comprises a first AB (comprising a first VL and a first VH) and a second AB (comprising a second VL and a second VH). In a specific embodiment, the first VL and the second VH are linked together by a linker, and the second VL and the first VH are linked together by a linker. In a further specific embodiment, the activatable antibody comprises a first single chain antibody comprising the first VL a first linker, and the first VH, and a second single chain antibody comprising the second VL, a second linker, and the second VH, wherein the first single chain antibody and the second single chain antibody are linked together by a third linker.

Activatable antibodies employed in the compositions and processes of the present invention may comprise any of a wide variety of CMs having an amino acid sequence that functions as a substrate for a protease. Suitable substrate amino acid sequences may be identified using any of a variety of known techniques including those described in U.S. Pat. Nos. 7,666,817, 8,563,269, PCT Publication No. WO 2014/026136, and Boulware et al. “Evolutionary optimization of peptide substrates for proteases that exhibit rapid hydrolysis kinetics,” Biotechnol Bioeng. 106.3 (2010): 339-46, each of which is incorporated by reference in their entireties.

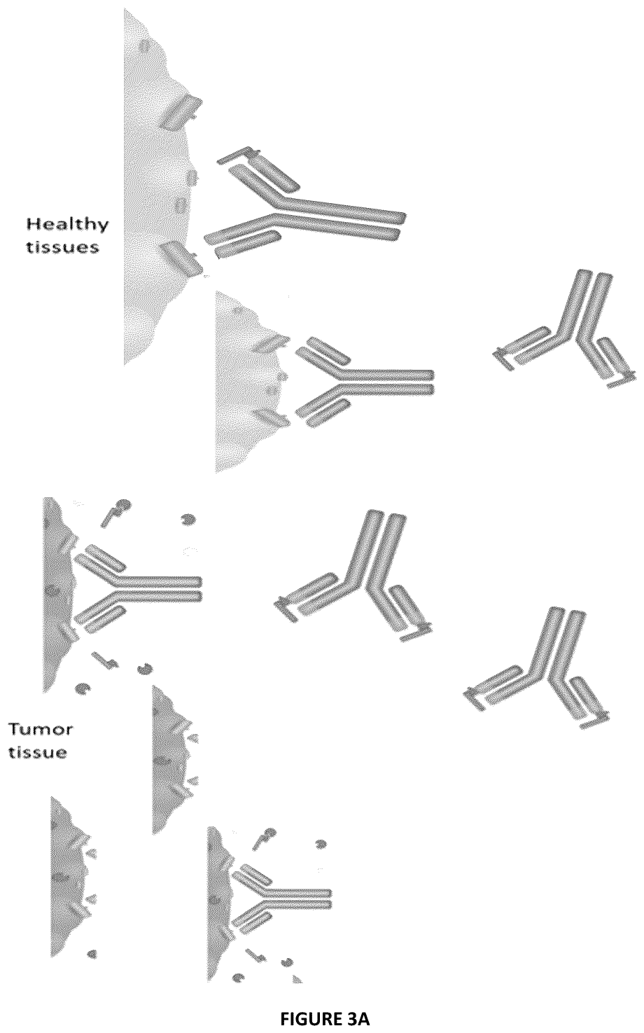

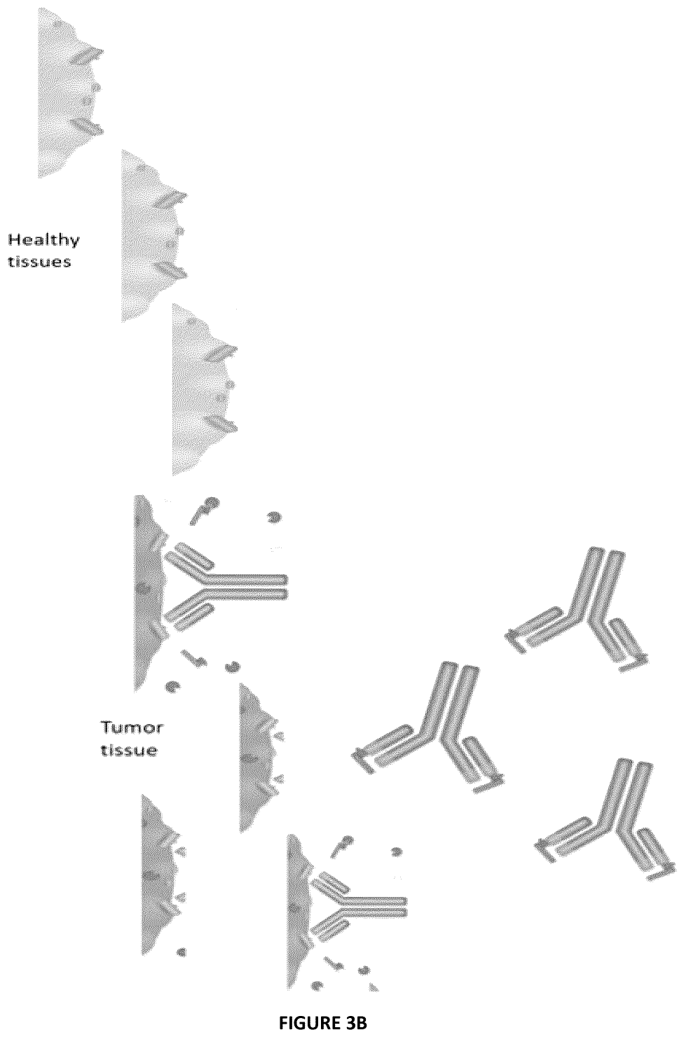

In some embodiments, the CM comprises a substrate for a protease that is active, e.g., up-regulated or otherwise unregulated, in a disease condition or diseased tissue, such that the protease cleaves the CM in the activatable antibody when the activatable antibody is exposed to the protease. Exemplary disease conditions include, for example, a cancer (e.g., where the diseased tissue is a tumor tissue) and an inflammatory or autoimmune condition (e.g., where the diseased tissue is inflamed tissue). In some embodiments, the CM comprises a substrate for an extracellular protease. As illustrated in FIGS. 3A and 3B, the CM of the intact activatable antibody of the present disclosure may be cleaved in the microenvironment of diseased tissues where proteases that cleave the CM are overexpressed relative to healthy tissue. Accordingly, the purified activatable antibody compositions of the present disclosure enable delivery of a target therapeutic dose of the intact activatable antibody to the relevant diseased tissue while minimizing the possibility of delivering a subtherapeutic dose to the diseased tissues and, additionally, avoids delivering clipped and thus, active antibody, to healthy tissues of the subject.

Suitable substrates may be readily identified using any of a variety of known techniques, including those described in U.S. Pat. Nos. 7,666,817, 8,563,269, PCT Publication No. WO 2014/026136, Boulware, et al., “Evolutionary optimization of peptide substrates for proteases that exhibit rapid hydrolysis kinetics,” Biotechnolo. Bioeng. (2010)106.3:339-46, each of which is hereby incorporated by reference in its entirety. Exemplary substrates include those that are substrates for any one or more of the following proteases: an ADAM, an ADAM-like, or ADAMTS (such as, for example, ADAM8, ADAM9, ADAM10, ADAM12, ADAM15, ADAM17/TACE, ADAMDEC1, ADAMTS1, ADAMTS4, ADAMTS5); an aspartate protease (such as, for example, BACE, Renin, and the like); an aspartic cathepsin (such as, for example, Cathepsin D, Cathepsin E, and the like); a caspase (such as, for example, Caspase 1, Caspase 2, Caspase 3, Caspase 4, Caspase 5, Caspase 6, Caspase 7, Caspase 7, Caspase 8, Caspase 9, Caspase 10, Caspase 14, and the like); a cysteine proteinase (such as, for example, Cruzipain, Legumain, Otubain-2, and the like); a kallikrein-related peptidase (KLK) (such as, for example, KLK4, KLK5, KLK6, KLK7, KLK8, KLK10, KLK11, KLK13, KLK14, and the like); a metallo proteinase (such as, for example, Meprin, Neprilysin, prostate-specific membrane antigen (PSMA), bone morphogenetic protein 1 (BMP-1), and the like); a matrix metalloproteinasc (MMP) (such as, for example, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, and the like); a serine protease (such as, for example, activated protein C, Cathepsin A, Cathepsin G, Chymase, a coagulation factor protease (such as, for example, FVIIa, FIXa, FXa, FXIa, FXIIa, and the like)); elastase, Granzyme B, Guanidinobenzoatase, HtrA1, Human Neutrophil Elastase, Lactoferrin, Marapsin, NS3/4A, PACE4, Plasmin, prostate-specific antigen (PSA), tissue plasminogen activator (tPA), Thrombin, Tryptase, urokinase (uPA), a Type II transmembrane Serine Protease (TTSP) (such as, for example, DESC1, DPP-4, FAP, Hepsin, Matriptase-2, MT-SP1/Matriptasc, TMPRSS2, TMPRSS3, TMPRSS4, and the like), and the like. Specific activatable antibody CMs are described, for example, in WO 2010/081173, WO 2015/048329, WO 2015/116933, and WO 2016/118629, each of which is incorporated herein by reference in its entirety.

In some embodiments, at least one or more of the CMs present in the intact activatable antibody (e.g., one or both of a first and second CM (if present)) comprises a substrate for at least one MMP. In certain of these embodiments, the MMP is selected from the group consisting of MMP9, MMP14, MMP1, MMP3, MMP13, MMP17, MMP11, and MMP19. In a specific embodiment, at least one or more of the CMs present in the intact activatable antibody (e.g., one or both of a first and second CM (if present)) comprises a substrate for MMP14. In certain embodiments, the CM is a substrate for at least one protease selected from the group consisting of a matrix metalloprotease (MMP), thrombin, a neutrophil elastase, a cysteine protease, legumain, and a serine protease, such as matriptase (MT-SP1), and urokinase (uPA). Without being bound by theory, it is believed that these proteases are up-regulated or otherwise unregulated in at least one of cancer, inflammation, or autoimmunity.

In some embodiments, one or more CMs present in the intact activatable antibody (e.g., one or both of a first and second CM (if present)) comprises an amino acid sequence comprising substrates for at least two different proteases (i.e., at least a first protease and at least a second protease). In these embodiments, the cleavage site may be the same or different for the at least two different proteases. Suitable first and second proteases include any of those described hereinabove. In certain of these embodiments, the first protease is selected from the group consisting of an MMP, a thrombin, a neutrophil elastase, a cysteine protease, a uPA, a legumain and a matriptase and the second protease is selected from the group consisting of any of those described hereinabove. In some embodiments, the first protease is a matrix metalloprotease (MMP) and the second protease is a serine protease (SP).

The CM may be designed to comprise two or more known substrates for the at least two or more different proteases, wherein the two or more substrates are covalently linked in series, either directly or directly, to each other, and wherein the CM is coupled to the AB. For example, CM may comprise a first substrate covalently linked either directly or indirectly, to a second substrate, where the first substrate is a substrate for a first protease, and the second substrate is a substrate for a second protease, in which the first and second protease are different proteases, and where the CM is coupled to the AB. In some embodiments, the first substrate and the second substrate are each a peptide of no more than about 25 amino acid residues long, or no more than about 24, or no more than about 23, or no more than about 22, or no more than about 21, or no more than about 20, or no more than about 19, or no more than about 18, or no more than about 17, or no more than about 15 amino acid residues long.

In some embodiments, the intact activatable antibody comprises at least one substrate for a protease selected from the group consisting of an MMP, thrombin, a neutrophil elastase, a cysteine protease, uPA, legumain, and matriptase. In some embodiments the activatable antibody comprises two prodomains, and hence, a first CM and a second CM. In certain of these embodiments, the first CM and the second CM comprise a substrate for the same protease. In some of these embodiments, the first CM comprises a first substrate and the second CM comprises a second substrate where the first substrate and the second substrate are substrates for two different proteases.

In certain embodiments, at least one CM in the activatable antibody (e.g., if a second prodomain is present in the activatable antibody, at least one of a first CM, and a second CM) comprises an amino acid sequence selected from the group consisting of SEQ ID NOs: 1-67. In some embodiments, each CM present in the activatable antibody (e.g., if a second prodomain is present in the activatable antibody, both of a first CM and a second CM) independently comprises an amino acid sequence selected from the group consisting of SEQ ID NOs: 1-67. In certain of these embodiments, each CM present in the activatable antibody comprises the same amino acid sequence (e.g., if a second prodomain is present in the activatable antibody, the first CM and the second CM comprise the same amino acid sequence).

The MM components of the activatable antibodies employed in the compositions and processes of the invention may be of any of a variety of lengths. In some embodiments, each MM present in the activatable antibody (e.g., a first MM; a first MM and a second MM; and the like) are each a peptide of no more than about 30 amino acid residues in length, or no more than about 29, or no more than about 28, or no more than about 27, or no more than about 26, or no more than about 25, or no more than about 24, or no more than about 23, or no more than about 22, or no more than about 21, or no more than about 20 amino acid residues in length. Each MM present reduces the ability of the corresponding AB to specifically bind to its respective biological target.

The MM can inhibit the binding of the activatable antibody to the biological target in a variety of ways. For example, the MM can bind to the AB thereby inhibiting binding of the activatable antibody to the biological target. The MM can allosterically or sterically inhibit binding of the activatable antibody to biological target. In some embodiments, the MM binds specifically to the AB. Suitable MMs may be identified using any of a variety of known techniques, including those described in U.S. Patent Application Publication Nos. 2009/0062142 and 2012/0244154, and PCT Publication No. WO 2014/026136, each of which is hereby incorporated by reference in their entirety.

Often, the dissociation constant (Kd) of the activatable antibody toward the biological target is greater than the Kd of the corresponding AB (absent the MM) toward the biological target.

In some embodiments, the MM reduces binding of the AB to the biological target by at least about 50%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, and even 100%, for at least about 2 hours, or at least about 4 hours, or at least about 6 hours, or at least about 8 hours, or at least about 12 hours, or at least about 24 hours, or at least about 28 hours, or at least about 30 hours, or at least about 36 hours, or at least about 48 hours, or at least about 60 hours, or at least about 72 hours, or at least about 84 hours, or at least about 96 hours, or at least about 5 days, or at least about 10 days, or at least about 15 days, or at least about 30 days, or at least about 45 days, or at least about 60 days, or at least about 90 days, or at least about 120 days, or at least about 150 days, or at least about 180 days, or at least about 1 month, or at least about 2 months, or at least about 3 months, or at least about 4 months, or at least about 5 months, or at least about 6 months, or at least about 7 months, or at least about 8 months, or at least about 9 months, or at least about 10 months, or at least about 11 months, or at least about 12 months or more. Illustrative MMs include those described in, for example, WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740, and U.S. Provisional Application Ser. Nos. 62/469,429, 62/572,467, each of which is incorporated herein by reference in their entireties.

Activatable antibodies employed in the practice of the present invention may contain one or more additional structural elements, such as, for example, one or more linkers, or other amino acid residue or peptide that may impart a structural benefit, such as an N-terminal spacer amino acid residue or amino acid sequence, and the like.

Linkers suitable for use in the activatable antibodies of the compositions and processes of the present invention may be any of a variety of lengths. Suitable linkers include those having a length in the range of from about 1 to about 20 amino acid residues, or from about 1 to about 19 amino acid residues, or from about 1 to about 18 amino acid residues, or from about 1 to about 17 amino acid residues, or from about 1 to about 16 amino acid residues, or from about 1 to about 15 amino acid residues, or from about 2 to about 15 amino acid residues, or from about 3 to about 15 amino acid residues, or from about 3 to about 14 amino acid residues, or from about 3 to about 13 amino acid residues, or from about 3 to about 12 amino acid residues. In some embodiments, the activatable antibody comprises one or more linkers each independently comprising 1, 2, 3, 4, 5, 6, 7, 8, 9, 10, 11, 12, 13, 14, 15, 16, 17, 18, 19, or 20 amino acid residues.

Typically, the linker is a flexible linker comprising one or more amino acid residues selected from the group consisting of Gly, Ser, Ala, and Thr, and often, the linker comprises one or more amino acid residues selected from the group consisting of Gly and Ser. Exemplary flexible linkers include a glycine homopolymer (G) n (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10); a glycine-serine co-polymer, including, for example, (GS) n (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10), (GSGGS) n (SEQ ID NO:68) (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10), (GGGS) n (SEQ ID NO:69) (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10); a linker that comprises or consists of glycine and serine residues, such as, for example, GGSG (SEQ ID NO: 70), GGSGG (SEQ ID NO:71), GSGSG (SEQ ID NO:72), GSGGG (SEQ ID NO:73), GSSGGSGGSGG (SEQ ID NO:74), GSSGGSGGSGGS (SEQ ID NO:75), GSSGGSGGSGGSGGGS (SEQ ID NO:76), GSSGGSGGSG (SEQ ID NO:77), GSSGGSGGSGS (SEQ ID NO:78), GGGS (SEQ ID NO:79), GSSG (SEQ ID NO:80), GGGSSGGSGGSGG (SEQ ID NO:81), GGGSG (SEQ ID NO:82), GGGSGG (SEQ ID NO: 152), GSGGGS (SEQ ID NO:153), GSGGSG (SEQ ID NO:154), GGS, and the like; a linker that comprises or consists of glycine, serine, and threonine residues, such as, for example, GSSGT (SEQ ID NO:83); a glycine-alanine co-polymer; an alanine-serine co-polymer; as well as other flexible linkers known in the art.

In some embodiments, activatable antibodies employed in the practice of the present invention may also comprise a spacer located, for example, at the amino terminus of the MM. In some embodiments, the spacer is joined directly to each MM of the activatable antibody, for example, in the structural arrangement, from N-terminus to C-terminus, of spacer-MM-CM-AB, wherein each “-” refers independently to a direct or indirect (i.e., via any of the linkers described herein). Illustrative spacer amino acid sequences may comprise or consist of any of the following exemplary amino acid sequences: QGQSGS (SEQ ID NO:84); GQSGS (SEQ ID NO: 85); QSGS (SEQ ID NO:86); SGS; GS; S; QGQSGQG (SEQ ID NO:87); GQSGQG (SEQ ID NO: 88); QSGQG (SEQ ID NO:89); SGQG (SEQ ID NO:90); GQG; QG; G; QGQSGQ (SEQ ID NO: 91); GQSGQ (SEQ ID NO:92); QSGQ (SEQ ID NO:93); QGQSG (SEQ ID NO:94); QGQS (SEQ ID NO:95); SGQ; GQ; and Q.

As described above, purified activatable antibody compositions may be prepared for any of a variety of activatable antibodies. Illustrative activatable antibodies may comprise a set of VL and VH CDRs that result in binding specificity for any of a number of biological targets. Illustrative specific CDRs include, for example, those in a VL and VH that together form an AB that has binding specificity for human CD166, in which the VL comprises a VL CDR1 having the amino acid sequence of SEQ ID NO:96, a VL CDR2 having the amino acid sequence of SEQ ID NO: 97, and a VL CDR3 having the amino acid sequence of SEQ ID NO:98, and the VH comprises a VH CDR1 having the amino acid sequence of SEQ ID NO:99, a VH CDR2 having the amino acid sequence of SEQ ID NO: 100, and a VH CDR3 having the amino acid sequence of SEQ ID NO:101. In these activatable antibodies, the MM may comprise an amino acid sequence selected from the group consisting of SEQ ID NOs: 102-119. A specific anti-CD166 activatable antibody comprising these CDRs comprises a first light chain and a second light chain that are identical, and a first heavy chain and a second heavy chain that are identical, wherein each of the first and second light chains comprise identical first and second VL amino acid sequences, identical first and second MMs, and identical first and second CMs, wherein each of the first and second light chains comprises the amino acid sequence of SEQ ID NO:120, and wherein each of the first and second heavy chains comprise an amino acid sequence selected from the group consisting of SEQ ID NO:121 and SEQ ID NO:122.

Another set of illustrative CDRs include those in a VL and VH that together form an AB that has binding specificity for human PD1, in which the VL comprises a VL CDR1 having the amino acid sequence of SEQ ID NO:123 or SEQ ID NO:129, a VL CDR2 having the amino acid sequence of SEQ ID NO: 124, and a VL CDR3 having the amino acid sequence of SEQ ID NO: 125, and the VH comprises a VH CDR1 having the amino acid sequence of SEQ ID NO: 126, a VH CDR2 having the amino acid sequence of SEQ ID NO:127, and a VH CDR3 having the amino acid sequence of SEQ ID NO:128. In these activatable antibodies, the MM may comprise an amino acid sequence selected from the group consisting of SEQ ID NOs: 130-280. A specific anti-PD1 activatable antibody comprising these CDRs comprises a first light chain and a second light chain that are identical, and a first heavy chain and a second heavy chain that are identical, wherein each of the first and second light chains comprise identical first and second VL amino acid sequences, identical first and second MMs, identical first and second CMs, and identical first and second light constant domains, wherein each of the first and second light chains comprises the amino acid sequence of SEQ ID NO:281, and wherein each of the first and second heavy chains comprise identical first and second VH amino acid sequences and identical first and second heavy constant domains, and wherein each of the first and second heavy chains comprise the amino acid sequence of SEQ ID NO:282.

Another illustrative set of CDRs include those in a VL and VH that together form an AB that has binding specificity for human PDL1, in which the VL comprises a VL CDR1 having the amino acid sequence of SEQ ID NO:283, a VL CDR2 having the amino acid sequence of SEQ ID NO: 284, and a VL CDR3 having the amino acid sequence of SEQ ID NO:285, and the VH comprises a VH CDR1 having the amino acid sequence of SEQ ID NO:286, a VH CDR2 having the amino acid sequence of SEQ ID NO:287, and a VH CDR3 having the amino acid sequence of SEQ ID NO:288. In these activatable antibodies, the MM may comprise an amino acid sequence selected from the group consisting of SEQ ID NOs: 289-313. A specific anti-PDL1 activatable antibody comprising these CDRs comprises a first light chain and a second light chain that are identical, and a first heavy chain and a second heavy chain that are identical, wherein each of the first and second light chains comprise identical first and second VL amino acid sequences, identical first and second MMs, identical first and second CMs, and identical first and second light constant domains, wherein each of the first and second light chains comprises the amino acid sequence of SEQ ID NO:314, and wherein each of the first and second heavy chains comprise identical first and second VH amino acid sequences and identical first and second heavy constant domains, and wherein each of the first and second heavy chains comprise the amino acid sequence of SEQ ID NO:315.

In another aspect, the present disclosure includes a method for determining or monitoring a relative quantity of an activatable antibody and a clipped variant thereof during a composition production process, by subjecting a sample composition comprising a population of activatable antibody and a population of clipped variants thereof to a gel capillary electrophoresis procedure. The gel capillary electrophoresis procedure may be reducing SDS-cGE or non-reducing SDS-cGE. In one aspect, the method involves separating the population of intact prodomain-encoding polypeptide from the population of clipped variants thereof using a reducing SDS-cGE procedure, and quantifying the relative amounts of the population of activatable antibody and the population of clipped variants thereof by determining the peak area corresponding to intact prodomain-encoding polypeptide and the peak area corresponding to the clipped prodomain-encoding polypeptide(s) thereof. In another aspect, the method involves separating the population of activatable antibody from the population of clipped variants thereof using a non-reducing SDS-cGE procedure, and quantifying the relative amounts of the population of activatable antibody and the population of clipped variants thereof by determining the peak area corresponding to intact activatable antibody and the peak area corresponding to clipped variant thereof.

In some aspects, the gel capillary electrophoresis method described herein can identify the presence of clipped variants that are not observed by other chromatography methods such as size exclusion chromatography (e.g., SE-HPLC), anion exchange chromatography, or cation exchange chromatography.

Thus, in some aspects, the quantitative gel capillary electrophoresis method described herein may be used to evaluate product and process quality for an activatable antibody, e.g., for quality control monitoring in a pharmaceutical production process. In some aspects, the method may be used to determine whether a purified composition comprising an activatable antibody is suitable for use to prepare a pharmaceutical composition. In some aspects, the present disclosure includes a method for preparing a pharmaceutical composition comprising an activatable antibody comprising a) providing a purified composition comprising intact activatable antibody and clipped variant thereof, b) separating intact activatable antibody species from clipped variants thereof by subjecting the sample to a reducing or non-reducing SDS-cGE procedure, c) quantifying the relative amounts of the population of activatable antibody and the population of clipped variants thereof by determining the peak area corresponding to intact activatable antibody or intact prodomain-containing polypeptide and the peak area corresponding to clipped antibody or clipped prodomain-containing polypeptide(s) thereof, and d) selecting the purified composition for use in the preparation of a pharmaceutical composition when the relative quantity of clipped variant in the sample is less than a threshold value. In some aspects, the threshold value is 3% clipped variant. In some aspects, the threshold value is 2.5%, 2%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, or 0.2%.

In some aspects, the present disclosure provides a method for determining or monitoring a relative percentage of an activatable antibody and a clipped variant thereof during a composition production process, the method including:
a) subjecting a sample composition comprising a population of activatable antibody and a population of clipped variants thereof to a gel capillary electrophoresis procedure;
b) separating the population of activatable antibody from the population of clipped variants thereof in the gel capillary electrophoresis procedure; and
c) quantifying the relative amounts of the population of activatable antibody and the population of clipped variants thereof by determining the peak area corresponding to intact prodomain-containing polypeptide and the peak area corresponding to clipped prodomain-containing polypeptide(s) thereof.

In some aspects, the sample composition is a cell harvest composition of a cell expressing the activatable antibody. In some aspects, the sample composition has been subjected to protein affinity chromatography. In some aspects, the sample composition has been subjected to ion exchange chromatography. In some aspects, the sample composition has been subjected to anion exchange chromatography. In some aspects, the sample composition has been subjected to cation exchange chromatography. In some aspects, the sample composition has been subjected to hydrophobic interaction chromatography. In some aspects, the sample composition has been subjected to multimodal chromatography. In some aspects, the relative percentage is monitored by quantifying the relative percentages of the population of activatable antibody and the population of clipped variants thereof after each of two or more of cell harvest, protein affinity chromatography, ion exchange chromatography, hydrophobic interaction chromatography, and multimodal chromatography. In some aspects, step a) includes contacting the activatable antibody and clipped variant thereof in the sample composition with a reducing agent. In some aspects, the reducing agent is dithioerythritol (DTE), dithiothreitol (DTT), beta-mercaptoethanol, or a combination thereof. In some aspects, step a) includes contacting the activatable antibody and clipped variant thereof in the sample composition with a denaturing agent. In some aspects, the denaturing agent is sodium dodecyl sulfate (SDS). In some aspects, the method further includes performing a gel capillary electrophoresis procedure using SDS protein molecular weight standards and producing a molecular weight standard calibration curve. In some aspects, step c) includes obtaining an electropherogram containing a peak corresponding to each of the activatable antibody and clipped variant thereof subjected to the gel capillary electrophoresis procedure. In some aspects, the method further includes performing peak integration for each peak corresponding to each of the activatable antibody and clipped variant thereof to obtain the peak area of clipped prodomain-encoding polypeptide and the peak area of intact prodomain-encoding polypeptide.

The following examples further illustrate the practice of the invention, but should not be construed as limiting its scope in any way.

### EXAMPLES

### Example 1

### Analytical Methods

### A. Total Protein Concentration

Total protein concentration of the aqueous feedstock and purified product was determined by UV spectroscopy. Samples of each eluate were drawn immediately after the separation step and frozen until use in the assays described herein, e.g., total protein, SDS-cGE, SE-HPLC, and HCP ELISA assays. Samples were serial diluted in ultrapure water and protein absorbance was measured using a UV spectrophotometer (Cary 60 UV-Vis Spectrophotometer, Agilent Technologies, Inc.) at a wavelength of 280 nm. Protein concentrations were determined in accordance with Beer-Lambert law at a wavelength of 280 nm using a calculated molar extinction coefficient based on amino acid sequence. See, e.g., Pace, et al., Protein Science (1995)4:2411.

Total protein yield was calculated as follows:

$$Total Protein Yield = \frac{{( volume \times protein concentration at 280 nm )}_{eluate}}{{( volume \times concentration at 280 nm )}_{aqueous feedstock}} \times 100$$

### B. Quantitation of Activatable Antibody by Reducing Gel Capillary Electrophoresis Using Sodium Dodecyl Sulfate (SDS-cGE)

The purity of activatable antibody compositions (% intact activatable antibody) was determined using sodium dodecyl sulfate-capillary gel electrophoresis (SDS-cGE) methodology and UV photodiode array (PDA) detection (PA 800 Plus Pharmaceutical Analysis System, Beckman Coulter, Inc., Brea, CA). Samples containing activatable antibody were reduced using 0.5 M 1,4-Dithioerythritol (Sigma Aldrich, Cat. No. D8255-5G), then heat denatured in the presence of sodium dodecyl sulfate (SDS). Once denatured, the samples were separated by size in a capillary containing SDS polymer matrix, which provides sieving selectivity for the electrophoresis separation.

The assay for IgG purity and heterogeneity were run in accordance with manufacturer's instructions using reagents that include SDS Protein Sizing Standards, buffers, and parts provided in the IgG Purity and Heterogeneity Assay Kit from SCIEX, as described in the manufacturer's instruction manual.

A molecular weight standard calibration curve was generated in accordance with manufacturer's instructions. Peak integration was performed and the electropherogram was inspected to identify the polypeptide peaks. Peaks corresponding to clipped prodomain-encoding polypeptide appears before the expected intact prodomain-encoding polypeptide peak.

Percent intact activatable antibody (i.e., % intact activatable antibody or “purity”) and percent clipped impurity are determined as follows:

$$\% intact activatable antibody = \frac{\% Peak {Area}_{intact}}{( \% Peak Area {,}_{clipped} + \% Peak Area {,}_{intact} )} \times 100$$

$$\% clipped impurity = \frac{\% Peak Area {,}_{clipped}}{( \% Peak Area {,}_{clipped} + \% Peak Area {,}_{intact} )} \times 100$$

$$where :$$

$$\% Peak Area {,}_{clipped} = \frac{( Peak Area of clipped prodomain - encoding polypeptide )}{( Total Peak Area corresponding to all species detected )} \times 100$$

$$\% Peak Area {,}_{intact} = \frac{( Peak Area of intact prodomain - encoding polypeptide )}{( Total Peak Area corresponding to all species detected )} \times 100$$

### C. Quantitation of HMWS by Size Exclusion High Performance Liquid Chromatography (SE-HPLC)

Size exclusion high performance liquid chromatography (SE-HPLC) was used to quantitate the amount of HMWS in the compositions described herein.

A mobile phase containing 200 mM K₂PO₄ and 150 mM KCl in UltraPure water was prepared (pH 6.8±0.1). Analysis was conducted on an HPLC system with autosampler (Dionex U3000 HPLC, Ultimate™ WPS-3000TBRS thermostatted biocompatible auto-sampler, Thermofisher Scientific) with column: analytical column (TSKgel G3000SWxl, Cat. No. 08541, Tosoh Bioscience LLC), guard column (TSKgel Guard SWx1, 6 mm×40 mm, Cat. No. 08543, Tosoh Bioscience LLC), and fritted pre-column (0.5 μm pre-column frit, Upchurch Scientific, Cat. No. A-318). The column was equilibrated with the mobile phase at a flow rate of 0.5 mL/min prior to sample analysis. Samples for analysis were diluted as necessary to a target concentration of 1 mg/mL with deionized or higher-grade water. Reference material was the corresponding monomeric activatable antibody (i.e., non-aggregated activated antibody). The reference material was diluted if necessary to a target concentration of 1 mg/mL with phosphate buffered saline (PBS).

Sample analysis was conducted at wavelengths of 220 nm (4 nm bandwidth) and 280 nm (4 nm bandwidth) for UV detection; ambient column oven temperature; 5°±3° C. sample temperature in auto-sampler; flow rate of 0.5 ml/min; and a run time of 30 min/sample. A formulation buffer blank injection was performed at the beginning of each series of samples prior to injection of the reference material. 20 μg of reference material was injected, followed by 40 μg of the test sample. 20 μg of the reference material was injected after each series of injection of each series of test samples.

The chromatograms of the test samples were integrated using Smart Cobra Wizard in Chromeleon 7 CDS (Thermo Scientific). Low molecular weight species (LMWS) is detected to the right side of the main (reference material) peak. Peak(s) corresponding to high molecular weight species (HMWS) is/are detected to the left side of the main peak. Relative peak area percent was calculated on the basis of the sum of all peak areas for all the peaks in the chromatogram (total peak area). The peak area of each HMWS peak observed was summed, and the sum was divided by the total peak area and multiplied by 100 to generate the percent peak area corresponding to HMWS (i.e., % HMWS).

### D. Host Cell Protein (HCP)

Quantitation of HCP (in ppm (ng/ml product)) was determined using a commercially available HCP ELISA Kit in accordance with the manufacturer's directions (CHO HCP ELISA Kit, (F550), Cygnus Technologies, Inc.).

### Example 2

### Comparative Example: Cation Exchange Chromatography

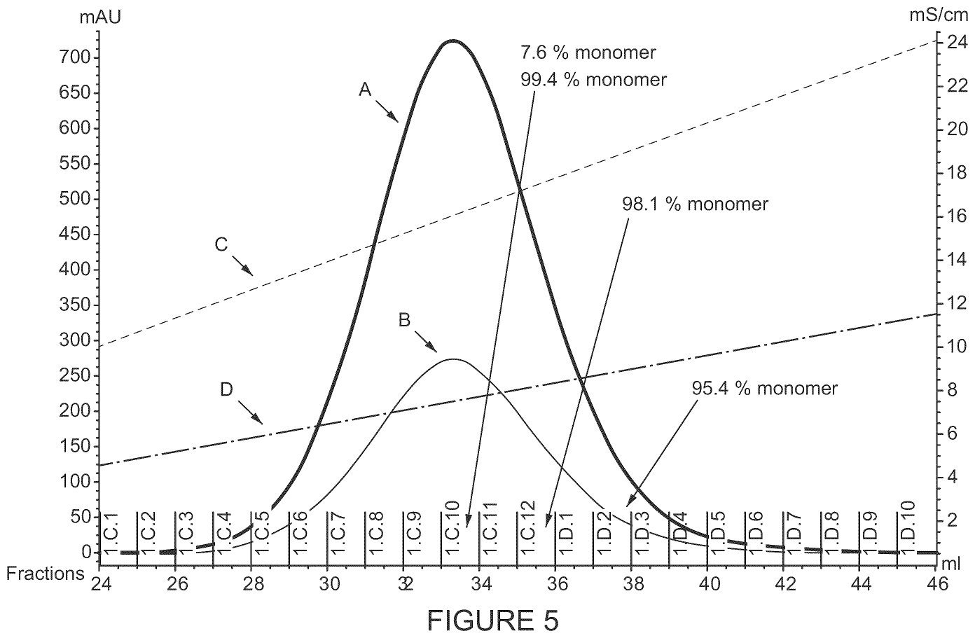

Cation exchange chromatography was evaluated as a method of purifying an aqueous feedstock comprising intact activatable anti-CD166 antibody and a corresponding clipped impurity. The % clipped impurity of the aqueous feedstock was greater than 7.5%. The aqueous feedstock comprised >98% monomer. The anti-CD166 activatable antibody has two light chains and two heavy chains. Each light chain encodes an MM, a CM, a VL, and a constant region, and each has the amino acid sequence of SEQ ID NO: 120. Each heavy chain encodes a VH and a constant region, and each has the amino acid sequence of SEQ ID NO: 121. The VH and VL of each pair of heavy chain and light chain together form a Fab that is capable of binding human CD166, when not masked. The aqueous feedstock had a total protein load of 10 mg/ml, as determined by absorbance at a wavelength of 280 nm as described in Example 1A. The aqueous feedstock was adjusted to pH 5.0 by a 1:10 dilution with equilibrium buffer, and loaded onto a cation exchange resin (Capto S ImpAct, GE Lifesciences). The column was eluted with a NaCl gradient at pH 5 by mixing a first buffer (25 mM NaOAc, 500 mM NaCl, PH 5.0 (80 CV grad)) into a second buffer (25 mM NaOAc, 25 mM NaCl, pH 5.0). 1 mL fractions were collected. A single peak was observed in the chromatogram from which 1 ml fractions were collected and analyzed by reducing SDS-cGE. FIG. 5 presents the resulting chromatogram (line “A” presents absorbance at 280 nm). The results indicated that both intact activatable antibody and clipped impurity were present, but no (peak) separation was achieved. Accordingly, cation exchange chromatography did not result in effective separation of intact activatable antibody from clipped variants.

### Example 3

### Preparation of a Purified Composition of an Activatable Antibody

An aqueous feedstock comprising the same anti-CD166 activatable antibody described in Example 2 was treated using the hydrophobic chromatography process of the present invention.

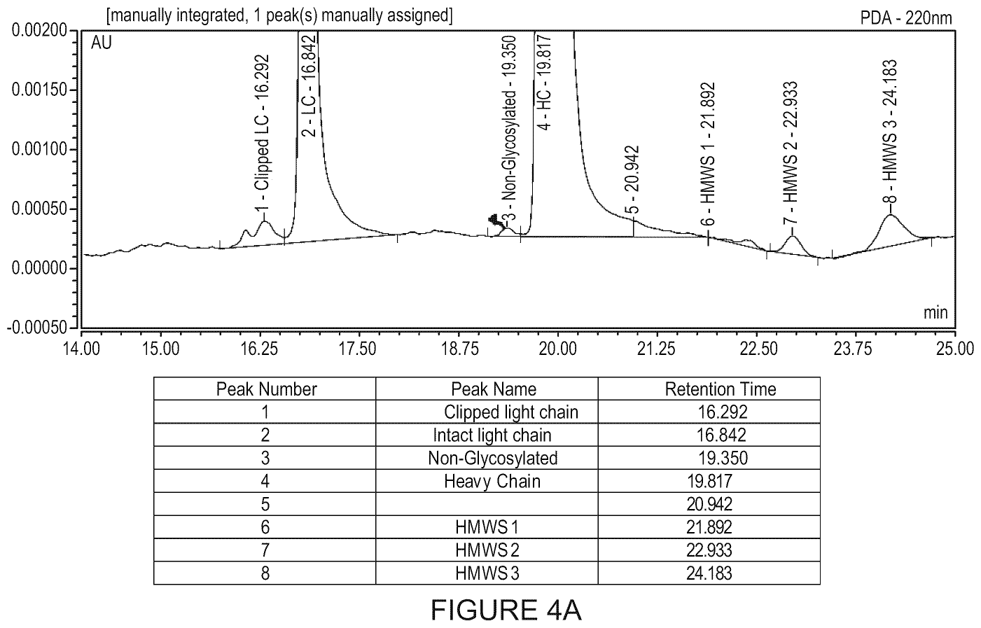

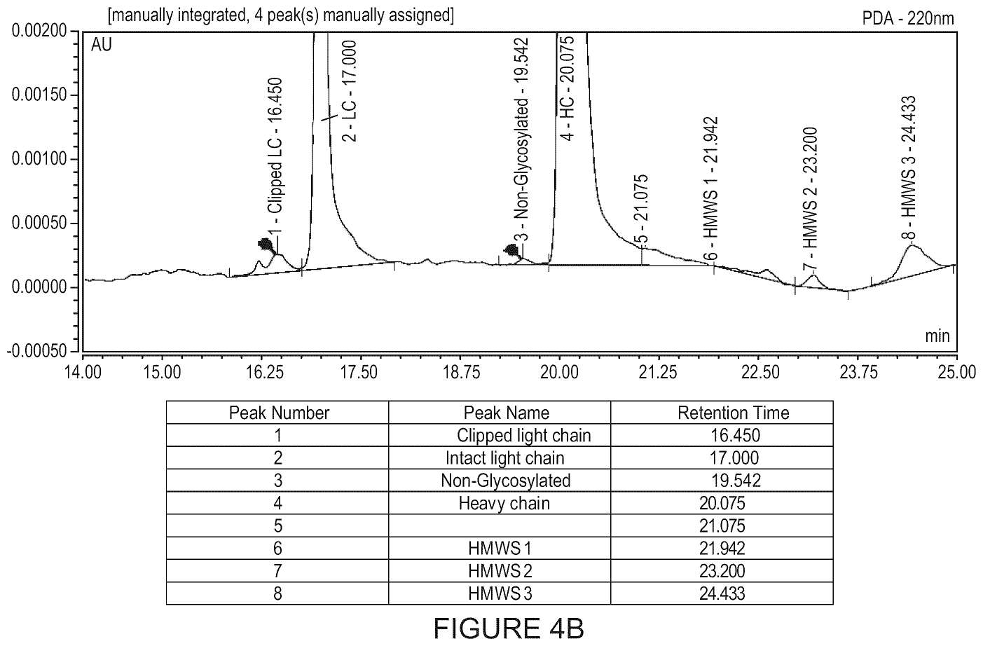

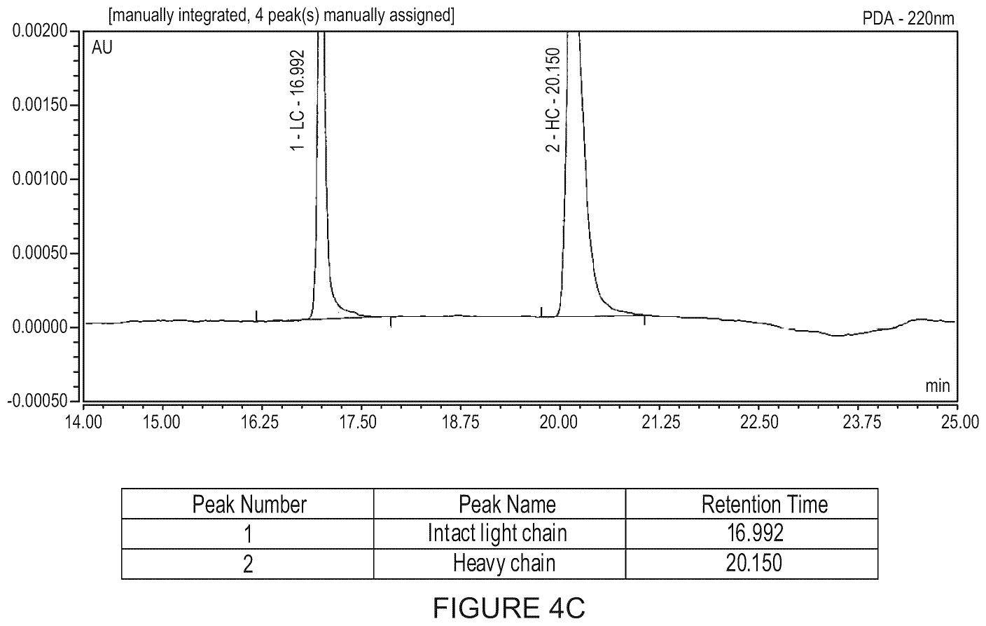

Bioreactor harvest supernatant comprising intact activatable antibody and clipped impurity was subjected to a Protein A affinity chromatography step, followed by a virus inactivation step, and an anion exchange chromatography (anion IEX) step. These processes did not result in effective separation of intact activatable antibody from clipped variants. FIG. 4A depicts the results of SDS-cGE analysis of a sample of the eluate from the Protein A affinity chromatography step. A significant peak comprising clipped variant is observed (Peak 1, “clipped LC”). FIG. 4B depicts the results of SDS-cGE analysis of a sample of the eluate from the anion IEX step. A significant peak comprising clipped variant is observed (Peak 1, “clipped LC”), and the peak area of the clipped variant is not reduced compared to the eluate from the earlier Protein A chromatography step. Product from the anion IEX step was collected for further processing in a hydrophobic chromatography process that utilized a chromatography column loaded with a hydrophobic interaction chromatography stationary phase (i.e., a hydrophobic interaction chromatography (HIC) column).

The HIC column was conducted in bind-and-elute mode, using Capto™ Phenyl ImpRes (GE Healthcare) as resin (i.e., the stationary phase). This resin is an agarose-based resin with phenyl-containing ligands. An aqueous feedstock was prepared by conditioning product collected from the anion IEX step with a buffer containing 20 mM 2-(N-morpholino) ethanesulfonic acid (MES) and 1.5 M ammonium sulfate (NH₄)₂SO₄, pH 6. The aqueous feedstock was split into two aliquots, “Aliquot 1” and “Aliquot 2”, for processing through the hydrophobic interaction chromatography column in two cycles, “Cycle 1” and “Cycle 2”, respectively. The chromatography equipment, including column and buffers were placed in a temperature cabinet maintained at 22° C.±1° C.

The two aliquots were loaded onto the column in two cycles. Load densities were set to a maximum of 20 mg/mL (resin) for Cycle 1, and 16 mg/mL (resin) for Cycle 2. The column was eluted with a buffer containing 20 mM MES and 0.26 M ammonium sulfate, pH 6.0. The peak cut criteria (UV cell with 2 mm path length) were set to 50 mAU upward inflection and 600 mAU downward inflection. Eluate was collected and the column was cleaned with a 1 M NaOH solution. Total protein concentration was determined by UV absorbance as described in Example 1, and total protein yields were calculated based on volume and protein concentrations in the aqueous feedstock and eluate. The total protein yields for the hydrophobic chromatography process step are provided in Table 1, below.

TABLE 1
Hydrophobic Chromatography Process (HIC)

| Sample | Yield (%, total protein) |
|---|---|
| Cycle 1 | >75% |
| Cycle 2 | >75% |

Relative quantities of intact activatable antibody and clipped impurity were determined by reducing SDS-cGE assays, as described in Example 1. The calculation of % intact activatable antibody and % clipped impurity was done on the basis of the light chain, which was the prodomain-encoding polypeptide.

A summary of process performance metrics for the hydrophobic chromatography process step is provided in Table 2, below.

TABLE 2
Hydrophobic Chromatography Step - Process Performance

| | | | | | Quantity of |
|---|---|---|---|---|---|
| | | | | | Clipped |
| | | | | | Impurity in |
| | % Intact | | | | Eluate as % of |
| | Activatable | % Clipped | Fold | | Clipped |
| | Antibody by | Impurity by | Reduction, | % Reduction | Impurity in |
| | reducing | reducing | Clipped | of Clipped | Aqueous |
| Sample | SDS-cGE | SDS-cGE | Impurity | Impurity | Feedstock |
| Feed to Anion | >93% | >4% | — | — | |
| IEX step | | | | | |
| Product of | >93% | >4% | No reduction | No reduction | ~100% |
| Anion | | | detected | detected | |
| IEX/Feedstock to | | | | | |
| HIC Cycles 1, 2 | | | | | |
| HIC Eluate, Cycle 1 | >95% | <1% | >7 | >85% | <13% |
| HIC Eluate, Cycle 2 | >95% | <1% | >9 | >88% | <11% |

The anion IEX step (immediately upstream of the hydrophobic chromatography step) appeared to have no measurable impact on reducing the amount of clipped impurity from intermediate composition. See FIGS. 4A and 4B. In contrast, the hydrophobic chromatography process step (using a HIC column) had a significant impact on removing clipped impurity. See FIG. 4C, which depicts the results of SDS-cGE analysis of a sample of the eluate from the hydrophobic chromatography step. No peak corresponding to clipped variant is observed in FIG. 4C. The quantity of clipped species in the eluate, as compared to that in the aqueous feedstock, indicated that the hydrophobic chromatography process was effective at reducing the quantity of clipped impurity in the process stream by about 8- to 9.6-fold. Residual levels of clipped impurity in each eluate was substantially less than that present in the aqueous feedstock for both cycles. The purity level (% intact activatable antibody) remained high in the eluate of both cycles, with residual clipped impurity levels in the eluate at less than 1% clipped impurity, as measured by reducing SDS-cGE.

Host cell protein levels in the aqueous feedstock and eluate compositions were characterized using the HCP ELISA analytical method described in Example 1. The results are shown below in Table 3. The quantity of HCP is reported in ppm (i.e., HCP in ng/mL (as determined by a corresponding HCP ELISA) divided by product concentration in mg/mL (as determined by absorbance at a wavelength of 280 nm as described in Example 1A)).

TABLE 3
Quantitation of Host Cell Protein

| | | | | Quantity of |
|---|---|---|---|---|
| | | | | HCP in Eluate, |
| | Host Cell | | | as % of HCP |
| | Protein | Fold | % | in Aqueous |
| Sample | (ppm) | Reduction | Reduction | Feedstock |
| HIC (aqueous) | >20 ppm | — | — | |
| Feedstock | | | | |
| to Cycle 1, | | | | |
| Cycle 2 | | | | |
| HIC Eluate, | <4 ppm | 7 | >85% | <15% |
| Cycle 1 | | | | |
| HIC Eluate, | <3 ppm | >7 | >85% | <15% |
| Cycle 2 | | | | |

The hydrophobic chromatography process step, using a HIC stationary phase, was effective at reducing HCP levels in the process stream by about 7-fold or more. Residual levels of HCP in the eluate were less than about 15% of feedstock levels.

HMWS levels were also assessed in the feedstock and eluate using the SE-HPLC assay described in Example 1. The results are shown below in Table 4.

TABLE 4
Quantitation of HMWS

| | | | | Quantity of |
|---|---|---|---|---|
| | | | | HMWS in |
| | | | | Eluate, as % of |
| | % HMWS | % | Fold | HMWS in |
| | (% Peak | Reduction | Reduction | Aqueous |
| Sample | Area) | of HMWS | of HMWS | Feedstock |
| HIC | >1 | — | — | — |
| Feedstock, | | | | |
| Cycle 1 | | | | |
| HIC | >1 | — | — | — |
| Feedstock, | | | | |
| Cycle 2 | | | | |
| HIC Eluate, | <1 | >85% | >7 | <15% |
| Cycle 1 | | | | |
| HIC Eluate, | <1 | >90% | >13 | <10% |
| Cycle 2 | | | | |

The results indicated that the process was effective in removing 85% or more of HMWS from the process stream. The resulting eluate compositions contained less than about 15% (Cycle 1) and 10% (Cycle 2) of the HMWS present in the aqueous feedstocks.

A summary of the relative quantities of intact activatable antibody, clipped impurity, HCP, and HMWS in the eluate (purified) compositions is provided in Table 5, below.

TABLE 5
Purified Compositions

| Eluate Composition | Eluate, Cycle 1 | Eluate, Cycle 2 |
|---|---|---|
| % Intact Activatable | >95% | >95% |
| Antibody* | | |
| % Clipped Impurity* | <1% | <1% |
| HCP (ppm) | <5 ppm | <5 ppm |
| (by HCP ELISA, as | | |
| described in Example 1) | | |
| % HMWS** | <0.5% | <0.5% |

*Determined by SDS-cGE, as described in Example 1, on the basis of % Peak Area corresponding to total light chain (intact and clipped).
**Determined by SE-HPLC, as described in Example 1.

Peak separation between the clipped impurity and the intact activatable antibody was achieved and resulted in successful separation of these species having very similar molecular weights and amino acid sequences. The results indicate that the hydrophobic chromatography process step was highly effective in generating compositions that are highly pure with respect to intact activatable antibody with low residual levels of clipped impurity, HCP, and HMWS.

### Example 4

Preparation of a Purified Composition of an Activatable Anti-PD1 Antibody Culture product comprising an anti-PD1 activatable antibody produced from two bioreactor runs (50 L scale (“Run 1) and 1,000 L scale (“Run 2”)) was purified to remove various impurities, including clipped impurity. The anti-PD1 activatable antibody has two light chains, each encoding an MM, a CM, a VL, and a constant region, and each having the amino acid sequence of SEQ ID NO:281, and two heavy chains, each encoding a VH and a constant region, and each having the amino acid sequence of SEQ ID NO:282. Each VH and VL together form a Fab that is capable of binding the target, human PD1, when not masked.

The downstream purification process for each bioreactor run was similar. Bioreactor harvest supernatant comprising the activatable anti-PD1 antibody product was subjected to a Protein A-based affinity chromatography step, followed by a virus inactivation step, and an anion exchange chromatography (anion IEX) step. These processes did not result in effective separation of intact activatable antibody from clipped variants. Product was collected for further processing in a HIC column. The conductivity of product from the anion IEX step was adjusted to 127.0±13 mS/cm for Run 1 and 126 mS/cm±13 for Run 2 with 20 mM MES and 1.8 M ammonium sulfate, pH 6.0. Product was divided into aliquots that were sized according to the load capacity of the HIC column. Each aliquot (aqueous feedstock) was subsequently loaded onto a HIC column (Capto™ Phenyl ImpRes, GE Healthcare) operated in bind/elute (B/E) mode at a temperature of 22±1° C. The column was eluted with an aqueous solution of 20 mM MES, 0.4 M ammonium sulfate, pH 6.0. The eluate was collected and the column was stripped with a 0.01M NaOH solution.

Total protein concentration was determined by UV absorbance as described in Example 1, and total protein yields were calculated based on volume and protein concentrations in the aqueous feedstock and eluate. The total protein yield for each aliquot applied to the HIC column is presented in Table 6, below.

TABLE 6
Hydrophobic Chromatography Process (HIC), Total Protein Yield

| | | Yield |
|---|---|---|
| Run | Aliquot | (%, total protein) |
| 1 | 1 | >80% |
| | 2 | >80% |
| 2 | 1 | >75% |
| | 2 | >75% |
| | 3 | >80% |
| | 4 | >80% |

The results indicate that total protein yields were relatively high for both runs.

Relative quantities of intact activatable antibody and clipped impurity were determined by reducing SDS-cGE assays as described in Example 1. The calculation of % intact activatable antibody and % clipped impurity was done on the basis of the light chain, which was the prodomain-encoding polypeptide.

A summary of process performance metrics for the hydrophobic chromatography process step for Run 1 and Run 2 is provided in Tables 7A and 7B, respectively, below. 10

TABLE 7A
Hydrophobic Chromatography Step - Process Performance, Run 1

| | | | | | Quantity of |
|---|---|---|---|---|---|
| | | | | | Clipped |
| | | | | | Species in |
| | % Intact | % Clipped | | | Eluate, as % of |
| | Activatable | Activatable | Fold | | Clipped |
| | Antibody by | Antibody by | Reduction, | % Reduction, | Impurity in |
| | Reducing | Reducing | Clipped | Clipped | Aqueous |
| Sample | SDS-cGE | SDS-cGE | Impurity | Impurity | Feedstock |
| Feed to Anion | >95% | >3% | — | — | — |
| IEX step | | | | | |
| Product of | >95% | >3% | No reduction | No reduction | ~100% |
| Anion | | | detected | detected | |
| IEX/Feed to | | | | | |
| HIC | | | | | |
| HIC Eluate, | >97% | <1% | >37 | >96% | <4%% |
| Run 1 | | | | | |
| HIC Eluate, | >97% | <1% | >18% | >94% | <6% |
| Run 2 | | | | | |

TABLE 7B
Hydrophobic Chromatography Step - Process Performance, Run 2

| | | | | | Quantity of |
|---|---|---|---|---|---|
| | | | | | Clipped |
| | | | | | Impurity in |
| | % Intact | | | | Eluate, as % of |
| | Activatable | % Clipped | Fold | | Clipped |
| | antibody by | Impurity by | Reduction, | % Reduction, | Impurity in |
| | Reducing | Reducing | Clipped | Clipped | Aqueous |
| Sample | SDS-cGE | SDS-cGE | Impurity | Impurity | Feedstock |
| Feed to Anion | >96% | >3% | — | — | — |
| IEX step | | | | | |
| Product of | >96% | >3% | No reduction | No reduction | ~100% |
| Anion | | | detected | detected | |
| IEX/Feedstock | | | | | |
| to HIC | | | | | |
| HIC Eluate, | >97% | <1% | >35 | >95% | <3% |
| Aliquot 1 | | | | | |
| HIC Eluate, | >97% | <1% | >17 | >93% | <6% |
| Aliquot 2 | | | | | |
| HIC Eluate, | >97% | <1% | >35 | >95% | <3% |
| Aliquot 3 | | | | | |
| HIC Eluate, | >97% | <1% | >17 | >93% | <6% |
| Aliquot 4 | | | | | |

As in the process scheme described in Example 3, the anion IEX step upstream of the hydrophobic chromatography step appeared to have no detectable impact on reducing the amount of clipped impurity from the intermediate composition. In contrast, the hydrophobic chromatography process step was highly effective at reducing the quantity of clipped impurity in the process stream. Intermediate compositions were also analyzed for the presence of HCP using the HCP ELISA assay described in Example 1. The results are shown in Tables 8A (Run 1) and 8B (Run 2).

TABLE 8A
Quantitation of Host Cell Protein (Run 1)

| | | | | HCP in Eluate |
|---|---|---|---|---|
| | Host Cell | | | as % of HCP |
| | Protein | Fold | % | in Aqueous |
| Sample | (ppm HCP) | Reduction | Reduction | Feedstock |
| HIC Feedstock | >4 ppm | — | — | |
| HIC Eluate, | <2 ppm | >4 | >75% | <25 |
| Aliquot 1 | | | | |
| HIC Eluate, | <2 ppm | >4 | >75% | <25 |
| Aliquot 2 | | | | |

TABLE 8B
Quantitation of Host Cell Protein (Run 2)

| | Host Cell | | | HCP in Eluate |
|---|---|---|---|---|
| | Protein | Fold | % | as % of HCP |
| Sample | (ppm HCP) | Reduction | Reduction | in Feedstock |
| HIC Feedstock | >3 ppm | — | — | — |
| HIC Eluate, | <1 ppm | >4 | >75% | <25% |
| Aliquot 1 | | | | |
| HIC Eluate, | <1 ppm | >4 | >75% | <25% |
| Aliquot 2 | | | | |
| HIC Eluate, | <1 ppm | >4 | >75% | <25% |
| Aliquot 3 | | | | |
| HIC Eluate, | <1 ppm | >4 | >75% | <25% |
| Aliquot 4 | | | | |

The results indicate that the hydrophobic chromatography process step was effective at reducing the amount of HCP in the process stream by at least 4 fold.

HMWS levels were also assessed in aqueous feedstock and eluate samples using the SE-HPLC assay described in Example 1. The HMWS quantitation results are provided in Tables 9A (Run 1) and 9B (Run 2).

TABLE 9A
Quantitation of HMWS, (Run 1)

| | | | | HMWS in Eluate |
|---|---|---|---|---|
| | | Fold | % | as % of HMWS |
| | HMWS | Reduction | Reduction | in Aqueous |
| Sample | (% Area) | of HMWS | of HMWS | Feedstock |
| Feed to HIC | >2% | — | — | — |
| HIC Eluate - | <1.5% | >2 | >55% | <45% |
| Aliquot 1 | | | | |
| HIC Eluate - | <1% | >2.5 | >65% | <35% |
| Aliquot 2 | | | | |

TABLE 9B
Quantitation of HMWS (Run 2)

| | | | | HMWS in Eluate |
|---|---|---|---|---|
| | | Fold | % | as % of HMWS |
| | HMWS | Reduction | Reduction | in Aqueous |
| Sample | (% Area) | of HMWS | of HMWS | Feedstock |
| Feed to HIC | >2% | — | — | — |
| HIC Eluate - | <1% | >2.5 | >65% | <35% |
| Aliquot 1 | | | | |
| HIC Eluate - | <1% | >2.5 | >65% | <35% |
| Aliquot 2 | | | | |
| HIC Eluate - | <1% | >2.5 | >65% | <35% |
| Aliquot 3 | | | | |
| HIC Eluate - | <1% | >4 | >75% | <25% |
| Aliquot 4 | | | | |

The results indicate that the hydrophobic chromatography process was highly effective at reducing levels of HMWS from the process stream.

A summary of the relative quantities of intact activatable antibody, clipped impurity, HCP, and HMWS in the purified compositions generated by the hydrophobic chromatography process of the present invention is provided in Table 10A (Run 1) and Table 10B (Run 2), below.

TABLE 10A
Purified Compositions, Run 1 (50 L)

| Eluate Composition | Eluate, Aliquot 1 | Eluate, Aliquot 2 |
|---|---|---|
| % Intact Activatable | >95% | >95% |
| Antibody* | | |
| % Clipped Impurity* | <1% | <1% |
| HCP (ppm) | <1.5 ppm | <1.5 ppm |
| (by HCP ELISA, as | | |
| described in Example 1) | | |
| % HMWS** | <1.5% | <1% |

*Determined by SDS-cGE, as described in Example 1, on the basis of % Peak Area corresponding to total light chain (intact and clipped).
**Determined by SE-HPLC, as described in Example 1.

TABLE 10B
Purified Compositions, Run 2 (1000 L)

| | Eluate, | Eluate, | Eluate, | Eluate, |
|---|---|---|---|---|
| Eluate Composition | Aliquot 1 | Aliquot 2 | Aliquot 3 | Aliquot 4 |
| % Intact Activatable | >95% | >95% | >95% | >95% |
| Antibody* | | | | |
| % Clipped impurity* | <1% | <1% | <1% | <1% |
| HCP (ppm) | <1 ppm | <1 ppm | <1 ppm | <1 ppm |
| (HCP ELISA) | | | | |
| % HMWS** | <1% | <1% | <1% | <1% |

*Determined by SDS-cGE, as described in Example 1, on the basis of % Peak Area corresponding to total light chain (intact and clipped).
**Determined by SE-HPLC, as described in Example 1.

The results indicate that the hydrophobic chromatography process of the present invention, in this case using a HIC column, was successful in removing clipped species, HCP, and HMWS from the process stream, thus generating highly pure compositions of intact activatable antibody.

### Example 5

### Preparation of a Purified Composition of an Anti-PDL1 Activatable Antibody

Culture product comprising an anti-PDL1 activatable antibody was treated using the hydrophobic chromatography process of the present invention to remove various impurities. The anti-PDL1 activatable antibody has two light chains, each encoding an MM, a CM, a VL, and a constant region, and each having the amino acid sequence of SEQ ID NO:314, and two heavy chains, each encoding a VH and a constant region, and each having the amino acid sequence of SEQ ID NO:315. Each VH and VL together form a Fab that is capable of binding the target human PDL1, when not masked.

Bioreactor harvest supernatant comprising intact activatable antibody and clipped impurity was subjected to a Protein A affinity chromatography step, followed by a virus inactivation step, and an anion IEX step. These processes did not result in effective separation of intact activatable antibody from clipped variants. Product from the anion IEX step was collected for further processing in a hydrophobic chromatography process that utilized a chromatography column loaded with a multimodal chromatography stationary phase (i.e., a multimodal chromatography (MMC) column).

The MMC column was conducted in bind-and-elute mode, using Capto™ MMC ImpRes (GE Healthcare) as resin (i.e., a stationary phase with N-benzoyl-homocysteine ligands) at a temperature of 22±4° C.

Intermediate product composition (pH 5.9) was conditioned with 25 mM MES, 30 mM NaCl, then split into two aliquots (aqueous feedstock) and processed in two cycles. The load density was 30 mg/mlᵣₑₛᵢₙ for each run. The column was washed, then eluted with 25 mM MES, 30 mM NaCl, 90 mM Arginine HCl at pH 5.9. The eluate was collected and the column was cleaned with a 1 M NaOH solution.

Total protein concentration was determined by UV absorbance, as described in Example 1, and total protein yields were calculated based on volume and protein concentrations in the aqueous feedstock and eluate. The total protein yields for the hydrophobic chromatography process step are provided in Table 11, below.

TABLE 11
Hydrophobic Chromatography Process (MMC)

| Sample | Yield (%, total protein) |
|---|---|
| MMC | — |
| Feedstock | |
| Eluate, | >75% |
| Aliquot 1 | |
| Eluate, | >75% |
| Aliquot 2 | |

The results indicate that relatively high total protein yields were obtained from the process.

Relative quantities of intact activatable antibody and clipped impurity were determined by reducing SDS-cGE, as described in Example 1. The results are summarized in Table 12.

TABLE 12
Hydrophobic Chromatography Step using MMC - Process Performance

| | | | | | Quantity of |
|---|---|---|---|---|---|
| | | | | | Clipped |
| | | | | | Impurity in |
| | % Intact | | | | Eluate, as % of |
| | Activatable | % Clipped | Fold | | Clipped |
| | Antibody by | Impurity by | Reduction, | % Reduction | Impurity in |
| | Reducing | Reducing | Clipped | of Clipped | Aqueous |
| Sample | SDS-cGE | SDS-cGE | Impurity | Impurity | Feedstock |
| Feed to Anion | >95% | >0.5% | — | — | — |
| IEX step | | | | | |
| Product of | >95% | >0.5% | No reduction | No reduction | ~100% |
| Anion | | | detected | detected | |
| IEX/Aqueous | | | | | |
| Feedstock to | | | | | |
| MMC | | | | | |
| MMC Eluate, | >98% | <0.5% | >8 | >85% | <12% |
| Aliquot 1 | | | | | |
| MMC Eluate, | >98% | <0.5% | >8 | >85% | <12% |
| Aliquot 2 | | | | | |

The results indicate that implementation of the hydrophobic chromatography process, in this case, using an MMC column, effectively removed more than 85% of clipped impurity from the process stream, resulting in a greater than 8-fold reduction of this impurity. Levels of intact activatable antibody (purity) were high in both eluant samples at greater than 98%. By contrast, the anion IEX step did not appear effective at reducing the level of clipped impurity in the process stream.

MMC feedstock and eluate compositions were also analyzed for the presence of HCP. The results are shown in Table 13.

TABLE 13
Quantitation of Host Cell Protein

| | | | | Quantity of |
|---|---|---|---|---|
| | | | | HCP in Eluate |
| | Host Cell | | | as % of HCP |
| | Protein | Fold | % | in MMC |
| Sample | (ppm HCP) | Reduction | Reduction | Feedstock |
| MMC Aqueous | >55 ppm | — | | — |
| Feedstock | | | | |
| MMC Eluate, | <9 ppm | >6 | >84% | <16% |
| Aliquot 1 | | | | |
| MMC Eluate, | <6 ppm | >11 | >90% | <9% |
| Aliquot 2 | | | | |

The results indicate that the hydrophobic chromatography step, using an MMC chromatography column, was effective at reducing the amount of HCP in the MMC feedstock composition by greater than 6-fold (greater than 84%) for Aliquot 1 and greater than 11-fold (greater than 90%) for Aliquot 2. Residual quantities of HCP in the eluate were less than 16% and about 9% of feedstock levels for Aliquots 1 and 2, respectively.

HMWS quantities were also assessed in feedstock and eluate samples using the SE-HPLC assay described in Example 1. The HMWS quantitation results are provided in Table 14, below.

TABLE 14
Quantitation of HMWS

| | | | | Quantity of |
|---|---|---|---|---|
| | | | | HMWS in |
| | | | | Eluate as % of |
| | | | | HMWS in |
| | % HMWS | Fold | % | Aqueous |
| Sample | (% Area) | Reduction | Reduction | Feedstock |
| Product of | >3% | — | — | — |
| Anion IEX | | | | |
| Step/Feed | | | | |
| to MMC | | | | |
| MMC | <1% | >4 | >75% | <25 |
| Eluate - | | | | |
| Aliquot 1 | | | | |
| MMC | <1% | >9 | >85% | <12 |
| Eluate - | | | | |
| Aliquot 2 | | | | |

The results indicate that the hydrophobic chromatography process step, in this case using an MMC column, was highly effective at removing a substantial fraction of HMWS from the process stream.

A summary of the relative quantities of intact activatable antibody, clipped impurity, HCP, and HMWS in the purified compositions generated by the hydrophobic chromatography process of the present invention is provided in Table 15, below.

TABLE 15
Purified Compositions

| Eluate Composition | Eluate, Run 1 | Eluate, Run 2 |
|---|---|---|
| % Intact Activatable | >98% | >98% |
| Antibody* | | |
| % Clipped impurity* | <0.5% | <0.5% |
| HCP (ppm) | <9 ppm | <5 ppm |
| (by HCP ELISA, as described | | |
| in Example 1) | | |
| % HMWS** | <1 | <1 |

*Determined by SDS-cGE, as described in Example 1, on the basis of % Peak Area corresponding to total light chain (intact and clipped).
**Determined by SE-HPLC, as described in Example 1.

The results indicate that the hydrophobic chromatography process step was highly effective in generating relatively pure compositions of intact activatable antibody that have, if at all present, low residual levels of clipped impurity, HCP, and HMWS.

### Example 6

### Preparation of a Purified Composition of an Activatable Anti-PDL1 Antibody from Aqueous Feedstock with High Levels of Clipped Impurity

### A. Aqueous Feedstock: 3.1% Clipped Impurity

Aqueous feedstock comprising the anti-PDL1 antibody described in Example 5 was loaded onto an MMC column. The composition of the aqueous feedstock included 3.1% clipped impurity and greater than 98% of the activatable antibody in monomeric form (i.e., less than 2% HMWS).

The MMC column was conducted in bind-and-elute mode, using Capto™ MMC ImpRes (GE Healthcare) as resin (i.e., a stationary phase with N-benzoyl-homocysteine ligands) at a temperature of 22±4° C. The aqueous feedstock was conditioned with 25 mM MES, 30 mM NaCl (pH 6). The column was washed with 25 mM MES, 30 mM NaCl, 20 mM Arginine HCl at pH 6, then eluted with 25 mM MES, 30 mM NaCl, 90 mM Arginine HCl at pH 6. The eluate was collected and the column was cleaned with a 1 M NaOH solution.

Total protein concentration was determined by UV absorbance, as described in Example 1, and total protein yield was calculated based on volume and protein concentrations in the aqueous feedstock and eluate. The level of clipped impurity in the resulting eluate was reduced by greater than 7.5-fold, such that less than about 13% of the clipped impurity in the aqueous feedstock remained in the eluate. The quantity of monomeric activatable antibody remained at greater than 98%. Total protein yield was greater than 65%.

### B. Aqueous Feedstock: 13.5% Clipped Impurity

Aqueous feedstock comprising the anti-PDL1 antibody described in Example 5 was loaded onto an MMC column (loaded with Capto™ ImpRes resin (GE Healthcare), and processed as described in part A, above. The composition of the aqueous feedstock included 13.5% clipped impurity and 96.8% of the activatable antibody in monomeric form (i.e., greater than 3% HMWS). The level of clipped impurity in the resulting eluate was reduced by greater than 7-fold (<14% of the clipped impurity in the aqueous feedstock) and the quantity of monomeric activatable antibody was greater than 98% (i.e., less than 2% HMWS). Total protein yield was greater than 65%.

The results from these experiments suggest that the hydrophobic chromatography process can be employed to purify aqueous feedstock with relatively high clipped impurity loads. The hydrophobic chromatography process results in a composition that is substantially depleted of clipped impurity.

The sequence listing is shown below in Table 16.

TABLE 16
Amino Acid Sequences

| SEQ ID | | |
|---|---|---|
| NO. | DESCRIPTION | SEQUENCE |
| 1 | CM | LSGRSDNH |
| 2 | CM | TGRGPSWV |
| 3 | CM | PLTGRSGG |
| 4 | CM | TARGPSFK |
| 5 | CM | NTLSGRSENHSG |
| 6 | CM | NTLSGRSGNHGS |
| 7 | CM | TSTSGRSANPRG |
| 8 | CM | TSGRSANP |
| 9 | CM | VHMPLGFLGP |
| 10 | CM | AVGLLAPP |
| 11 | CM | AQNLLGMV |
| 12 | CM | QNQALRMA |
| 13 | CM | LAAPLGLL |
| 14 | CM | STFPFGMF |
| 15 | CM | ISSGLLSS |
| 16 | CM | PAGLWLDP |
| 17 | CM | VAGRSMRP |
| 18 | CM | VVPEGRRS |
| 19 | CM | ILPRSPAF |
| 20 | CM | MVLGRSLL |
| 21 | CM | QGRAITFI |
| 22 | CM | SPRSIMLA |
| 23 | CM | SMLRSMPL |
| 24 | CM | ISSGLLSGRSDNH |
| 25 | CM | AVGLLAPPGGLSGRSDNH |
| 26 | CM | ISSGLLSSGGSGGSLSGRSDNH |
| 27 | CM | LSGRSGNH |
| 28 | CM | SGRSANPRG |
| 29 | CM | LSGRSDDH |
| 30 | CM | LSGRSDIH |
| 31 | CM | LSGRSDQH |
| 32 | CM | LSGRSDTH |
| 33 | CM | LSGRSDYH |
| 34 | CM | LSGRSDNP |
| 35 | CM | LSGRSANP |
| 36 | CM | LSGRSANI |
| 37 | CM | LSGRSDNI |
| 38 | CM | MIAPVAYR |
| 39 | CM | RPSPMWAY |
| 40 | CM | WATPRPMR |
| 41 | CM | FRLLDWQW |
| 42 | CM | ISSGL |
| 43 | CM | ISSGLLS |
| 44 | CM | ISSGLL |
| 45 | CM | ISSGLLSGRSANPRG |
| 46 | CM | AVGLLAPPTSGRSANPRG |
| 47 | CM | AVGLLAPPSGRSANPRG |
| 48 | CM | ISSGLLSGRSDDH |
| 49 | CM | ISSGLLSGRSDIH |
| 50 | CM | ISSGLLSGRSDQH |
| 51 | CM | ISSGLLSGRSDTH |
| 52 | CM | ISSGLLSGRSDYH |
| 53 | CM | ISSGLLSGRSDNP |
| 54 | CM | ISSGLLSGRSANP |
| 55 | CM | ISSGLLSGRSANI |
| 56 | CM | AVGLLAPPGGLSGRSDDH |
| 57 | CM | AVGLLAPPGGLSGRSDIH |
| 58 | CM | AVGLLAPPGGLSGRSDQH |
| 59 | CM | AVGLLAPPGGLSGRSDTH |
| 60 | CM | AVGLLAPPGGLSGRSDYH |
| 61 | CM | AVGLLAPPGGLSGRSDNP |
| 62 | CM | AVGLLAPPGGLSGRSANP |
| 63 | CM | AVGLLAPPGGLSGRSANI |
| 64 | CM | ISSGLLSGRSDNI |
| 65 | CM | AVGLLAPPGGLSGRSDNI |
| 66 | CM | GLSGRSDNHGGAVGLLAPP |
| 67 | CM | GLSGRSDNHGGVHMPLGFLGP |
| 68 | Linker | GSGGS |
| 69 | Linker | GGGS |
| 70 | Linker | GGSG |
| 71 | Linker | GGSGG |
| 72 | Linker | GSGSG |
| 73 | Linker | GSGGG |
| 74 | Linker | GSSGGSGGSGG |
| 75 | Linker | GSSGGSGGSGGS |
| 76 | Linker | GSSGGSGGSGGSGGGS |
| 77 | Linker | GSSGGSGGSG |
| 78 | Linker | GSSGGSGGSGS |
| 79 | Linker | GGGS |
| 80 | Linker | GSSG |
| 81 | Linker | GGGSSGGSGGSGG |
| 82 | Linker | GGGSG |
| 83 | Linker | GSSGT |
| 84 | Spacer | QGQSGS |
| 85 | Spacer | GQSGS |
| 86 | Spacer | QSGS |
| 87 | Spacer | QGQSGQG |
| 88 | Spacer | GQSGQG |
| 89 | Spacer | QSGQG |
| 90 | Spacer | SGQG |
| 91 | Spacer | QGQSGQ |
| 92 | Spacer | GQSGQ |
| 93 | Spacer | QSGQ |
| 94 | Spacer | QGQSG |
| 95 | Spacer | QGQS |
| 96 | VL CDR1 | RSSKSLLHSNGITYLY |
| 97 | VL CDR2 | QMSNLAS |
| 98 | VL CDR3 | AQNLELPYT |
| 99 | VH CDR1 | GFSLSTYGMGVG |
| 100 | VH CDR2 | NIWWSEDKH |
| 101 | VH CDR3 | IDYGNDYAFTY |
| 102 | MM | LCHPLVLSAWESCSS |
| 103 | MM | LCHPAVLSAWESCSS |
| 104 | MM | LCHPLVASAWESCSS |
| 105 | MM | LEGWCLHPLCLWGAG |
| 106 | MM | LCAPLVLSAWESCSS |
| 107 | MM | LCHALVLSAWESCSS |
| 108 | MM | LCHPLALSAWESCSS |
| 109 | MM | LCHPLVLSAAESCSS |
| 110 | MM | LCHPLVLSAWASCSS |
| 111 | MM | HPLVL |
| 112 | MM | LEGACLHPLCLWGAG |
| 113 | MM | LEGWCAHPLCLWGAG |
| 114 | MM | LEGWCLAPLCLWGAG |
| 115 | MM | LEGWCLHACLWGAG |
| 116 | MM | LEGWCLHPACLWGAG |
| 117 | MM | LEGWCLHPLCAWGAG |
| 118 | MM | LEGWCLHPLCLAGAG |
| 119 | MM | CLHPLC |
| 120 | Human αCD166 | QGQSGQGLCHPAVLSAWESCSSG |
| | CX-191/CX-2009 | GGSSGGSAVGLLAPPGGLSGRSDN |
| | Light Chain | HGGSDIVMTQSPLSLPVTPGEPASI |
| | (spacer-MM-LP1- | SCRSSKSLLHSNGITYLYWYLQKP |
| | CM-LP2-Ab) | GQSPQLLIYQMSNLASGVPDRFSG |
| | | SGSGTDFTLKISRVEAEDVGVYYC |
| | | AQNLELPYTFGQGTKLEIKRTVAA |
| | | PSVFIFPPSDEQLKSGTASVVCLLN |
| | | NFYPREAKVQWKVDNALQSGNSQ |
| | | ESVTEQDSKDSTYSLSSTLTLSKAD |
| | | YEKHKVYACEVTHQGLSSPVTKSF |
| | | NRGEC |
| 121 | Human αCD166 | QITLKESGPTLVKPTQTLTLTCTFS |
| | Heavy Chain | GFSLSTYGMGVGWIRQPPGKALE |
| | (HuCD166_HcCD | WLANIWWSEDKHYSPSLKSRLTIT |
| | des-HC)) | KDTSKNQVVLTITNVDPVDTATYY |
| | | CVQIDYGNDYAFTYWGQGTLVTV |
| | | SSASTKGPSVFPLAPSSKSTSGGTA |
| | | ALGCLVKDYFPEPVTVSWNSGAL |
| | | TSGVHTFPAVLQSSGLYSLSSVVT |
| | | VPSSSLGTQTYICNVNHKPSNTKV |
| | | DKKVEPKSCDKTHTCPPCPAPELL |
| | | GGPSVFLFPPKPKDTLMISRTPEVT |
| | | CVVVDVSHEDPEVKFNWYVDGVE |
| | | VHNAKTKPREEQYNSTYRVVSVL |
| | | TVLHQDWLNGKEYKCKVSNKALP |
| | | APIEKTISKAKGQPREPQVYTLPPS |
| | | REEMTKNQVSLTCLVKGFYPSDIA |
| | | VEWESNGQPENNYKTTPPVLDSD |
| | | GSFFLYSKLTVDKSRWQQGNVFSC |
| | | SVMHEALHNHYTQKSLSLSPG |
| 122 | Human αCD166 | QITLKESGPTLVKPTQTLTLTCTFS |
| | Heavy Chain | GFSLSTYGMGVGWIRQPPGKALE |
| | (HuCD166_HcC) | WLANIWWSEDKHYSPSLKSRLTIT |
| | | KDTSKNQVVLTITNVDPVDTATYY |
| | | CVQIDYGNDYAFTYWGQGTLVTV |
| | | SSASTKGPSVFPLAPSSKSTSGGTA |
| | | ALGCLVKDYFPEPVTVSWNSGAL |
| | | TSGVHTFPAVLQSSGLYSLSSVVT |
| | | VPSSSLGTQTYICNVNHKPSNTKV |
| | | DKKVEPKSCDKTHTCPPCPAPELL |
| | | GGPSVFLFPPKPKDTLMISRTPEVT |
| | | CVVVDVSHEDPEVKFNWYVDGVE |
| | | VHNAKTKPREEQYNSTYRVVSVL |
| | | TVLHQDWLNGKEYKCKVSNKALP |
| | | APIEKTISKAKGQPREPQVYTLPPS |
| | | REEMTKNQVSLTCLVKGFYPSDIA |
| | | VEWESNGQPENNYKTTPPVLDSD |
| | | GSFFLYSKLTVDKSRWQQGNVFSC |
| | | SVMHEALHNHYTQKSLSLSPGK |
| 123 | CX-188 | RASESVDAYGISFMN |
| | 1.5 VL CDR1 | |
| 124 | 1.5 VL CDR2 | AASNQGS |
| 125 | 1.5 VL CDR3 | QQSKDVPWT |
| 126 | 1.5 VH CDR1 | GFTFSGYAMS |
| 127 | 1.5 VH CDR2 | YISNSGGNAH |
| 128 | 1.5 VH CDR3 | EDYGTSPFVY |
| 129 | 1.4 VL CDR1 | RASESVDSYGISFMN |
| 130 | PD001-Mask | AMSGCSWSAFCPYLA |
| 131 | PD002 | DVNCAIWYSVCTTVP |
| 132 | PD003 | LVCPLYALSSGVCMG |
| 133 | PD004 | SVNCRIWSAVCAGYE |
| 134 | PD005 | MLVCSLQPTAMCERV |
| 135 | PD006 | APRCYMFASYCKSQY |
| 136 | PD007 | VGPCELTPKPVCNTY |
| 137 | PD008 | ETCNQYERSSGLCFA |
| 138 | PD009 | APRTCYTYQCSSFYT |
| 139 | PD010 | GLCSWYLSSSGLCVD |
| 140 | PD011 | VPWCQLTPRVMCMWA |
| 141 | PD012 | NWLDCQFYSECSVYG |
| 142 | PD013 | SCPLYVMSSFGGCWD |
| 143 | PD014 | MSHCWMFSSSCDGVK |
| 144 | PD015 | VSYCTWLIEVTCLRG |
| 145 | PD016 | VLCAAYALSSGICGG |
| 146 | PD017 | TTCNLYQQSSMFCNA |
| 147 | PD018 | APRCYMFASYCKSQY |
| 148 | PD019 | PCDQNPYFYPYVCHA |
| 149 | PD020 | SVCPMYALSSMLCGA |
| 150 | PD021 | LSVECYVFSRCSSLP |
| 151 | PD022 | FYCTYLVSLTCHPQ |
| 152 | Linker | GGGSGG |
| 153 | Linker | GSGGGS |
| 154 | Linker | GSGGSG |
| 155 | PD023 | SMAGCQWSSFCVQRD |
| 156 | PD024 | IYSCYMFASRCTSDK |
| 157 | PD025 | SRCSVYEVSSGLCDW |
| 158 | PD026 | GMCSAYAYSSKLCTI |
| 159 | PD027 | MTTNTCNLLCQQFLT |
| 160 | PD028 | FQPCLMFASSCFTSK |
| 161 | PD029 | WNCHPAGVGPVFCEV |
| 162 | PD030 | ALCSMYLASSGLCNK |
| 163 | PD031 | NYLSCQFFQNCYETY |
| 164 | PD032 | GWCLFSDMWLGLCSA |
| 165 | PD033 | EFCARDWLPYQCSSF |
| 166 | PD034 | TSYCSIEHYPCNTHH |
| 167 | PD035 | PYICSSFPLDCQAGQ |
| 168 | PD036 | VGCEWYMSSSGMCSR |
| 169 | PD037 | EVCGGCSMQSVSCWP |
| 170 | PD038 | FTECQLSPKAICMSN |
| 171 | PD039 | KYCLFSEYVEGTCLN |
| 172 | PD040 | SGCPMYAWGWDECWR |
| 173 | PD041 | VDCPWYASSSAICSR |
| 174 | PD042 | DMLLCQIRGSCAAWG |
| 175 | PD043 | ECHPYQASASLWCGY |
| 176 | PD044 | MMMGCMWSAWCPPSR |
| 177 | PD045 | NAYFRCSLMCNMIMF |
| 178 | PD046 | ACCKESVHSVHDCKR |
| 179 | PD047 | ACIGINSYMSNYCYL |
| 180 | PD048 | ANCSFLELTNKFCTI |
| 181 | PD049 | AYCSYLMFASNPCII |
| 182 | PD050 | CFTSKCPCLCYSLLA |
| 183 | PD051 | CLCRDINCWLGCSKT |
| 184 | PD052 | CWCDIYCSPYQCSSF |
| 185 | PD053 | DCIYYYQQSANLCSY |
| 186 | PD054 | DCTGVNYYIDKHCTN |
| 187 | PD055 | DECHGYLRSSGLCGG |
| 188 | PD056 | DICSAYAASSGFCYY |
| 189 | PD057 | DIICVLTPTAWCGRT |
| 190 | PD058 | DNCCMYCSWWIACRD |
| 191 | PD059 | DSCQWYMLSADLCGT |
| 192 | PD060 | DSVCFSSSSFLCHKS |
| 193 | PD061 | DTMCAIWWTVCSGGR |
| 194 | PD062 | ECTYQTSSFHEACMS |
| 195 | PD063 | EGCNLYERSSYGCNN |
| 196 | PD064 | EGCTAFAMSAGICGG |
| 197 | PD065 | EQSCSLTPIAFCWSE |
| 198 | PD066 | EWCNAYISSSKLCST |
| 199 | PD067 | FEVCYMFASACRNGM |
| 200 | PD068 | FSCSWYAESSSLCDI |
| 201 | PD069 | FVCQMFEASSGLCGG |
| 202 | PD070 | FYCPCCMFASSCGSR |
| 203 | PD071 | FYCSYLPGASHQCSH |
| 204 | PD072 | FYCSYLYMCEVCCYE |
| 205 | PD073 | GFCTQHTVLTWCPTS |
| 206 | PD074 | GSCPSYAVSAGLCYA |
| 207 | PD075 | GSQCFLTPTAFCTHT |
| 208 | PD076 | GTCHPYMQSSKICNN |
| 209 | PD077 | GVECFVFTGGCGGYG |
| 210 | PD078 | HELCNGHWVPCCWAY |
| 211 | PD079 | ICDSYYAVSSGLCLL |
| 212 | PD080 | IGCAWYVSSAGWCSP |
| 213 | PD081 | INLCWMFASECGEHH |
| 214 | PD082 | KCWLAEMTNLEHCNM |
| 215 | PD083 | KHCSDFAYSSRLCDR |
| 216 | PD084 | KVCSSYASSSGLCGW |
| 217 | PD085 | LDSCYMFASYCVQAV |
| 218 | PD086 | LLACHPIFVTVCQTR |
| 219 | PD087 | LLSCPYNPEHVCHTS |
| 220 | PD088 | LMCSLYALSSNLCGR |
| 221 | PD089 | LMWCVLFLWSWCCRI |
| 222 | PD090 | LPICHLTPTAVCTHI |
| 223 | PD091 | LSNMCLAFGSCLYAW |
| 224 | PD092 | LSRCHPIWYTICQNP |
| 225 | PD093 | LTQCMSVHKECGGYE |
| 226 | PD094 | LVNCRIWSWVCEEAT |
| 227 | PD095 | LYCSWYQMSSAVCKE |
| 228 | PD096 | MECGWYALSARFCEV |
| 229 | PD097 | MTCSPYAMSAHFCNE |
| 230 | PD098 | MVCSLYAYSASLCGA |
| 231 | PD099 | NALCWSTFSWWCDMD |
| 232 | PD-100 | NFTCMLTPKAYCVQT |
| 233 | PD-101 | NGACIFTLSWCTNKT |
| 234 | PD-102 | NGCELYAAASGLCRT |
| 235 | PD-103 | NIECSVFGRCCCDNY |
| 236 | PD-104 | PACRPMFWNRSCDNI |
| 237 | PD-105 | PCRVSNMFFPYNCLD |
| 238 | PD-106 | PIMCMLLPESYCWIW |
| 239 | PD-107 | PQSCYMFASLCMPNG |
| 240 | PD-108 | PRCPQGLPLYQCSSF |
| 241 | PD-109 | PSVECLVFKRCYALP |
| 242 | PD-110 | PVCQRSATIYNCNWF |
| 243 | PD-111 | QCAAYYISSFGGCSN |
| 244 | PD-112 | QFGCFMLARDFCGTY |
| 245 | PD-113 | QMMCPYNPEHKCHQK |
| 246 | PD-114 | QRECWMFASSCNSKN |
| 247 | PD-115 | QSNMCTTYICSSFNY |
| 248 | PD-116 | QSRCHSLAPYLCSSF |
| 249 | PD-117 | RAYCSLLFADSCNNN |
| 250 | PD-118 | RCIGINQYIDSNCYN |
| 251 | PD-119 | RLSCFMFASQCALEF |
| 252 | PD-120 | RQCIILMNHRQCFFK |
| 253 | PD-121 | RSCTPYMMSSSLCNT |
| 254 | PD-122 | RYCHYWKMPYECSSF |
| 255 | PD-123 | SCVSLSWFDMLKCYE |
| 256 | PD-124 | SDNCEIWWTVCSAAM |
| 257 | PD-125 | SFCWSYLVSSGLCGV |
| 258 | PD-126 | SMCMNNYGTTIIVICGN |
| 259 | PD-127 | SMVGCGWSTFCPSRG |
| 260 | PD-128 | SSLHCANGHTCPFCL |
| 261 | PD-129 | SVCSYYEESSGICSP |
| 262 | PD-130 | SWCGWYAASSGVCAL |
| 263 | PD-131 | TCISQTIDSYLNCVN |
| 264 | PD-132 | TFCNLYTKSSNICMS |
| 265 | PD-133 | TYCVFHEYLDNTCNN |
| 266 | PD-134 | VATGCPNLMLCGSWP |
| 267 | PD-135 | VEYCSLLLGNRCDYW |
| 268 | PD-136 | VGCNMYLMSAGLCVD |
| 269 | PD-137 | VLYCSWDSGTCVGSH |
| 270 | PD-138 | VMFSCYYLETCAPGV |
| 271 | PD-139 | VRIGLCPESCLVSGF |
| 272 | PD-140 | VTCTYYATSSSLCNT |
| 273 | PD-141 | VTGCILLPKAWCWGD |
| 274 | PD-142 | VWCSIYEYSSNLCSR |
| 275 | PD-143 | WMLECQYNNTCNNMT |
| 276 | PD-144 | WPCSPLEYYNNICNV |
| 277 | PD-145 | WTYDCHLNQTCPTYY |
| 278 | PD-146 | YCSINMYLIGGNCMY |
| 279 | PD-147 | YFCSLYANSAGFCGG |
| 280 | PD-148 | YVSCYMFSSSCPSTW |
| 281 | CX-188 LC | QGQSGQGTSYCSIEHYPCNTHHGG |
| | | GSSGGSISSGLLSGRSDNPGGGSDI |
| | | QLTQSPSSLSASVGDRVTITCRASE |
| | | SVDAYGISFMNWFQQKPGKAPKL |
| | | LIYAASNQGSGVPSRFSGSGSGTDF |
| | | TLTISSMQPEDFATYYCQQSKDVP |
| | | WTFGQGTKLEIKRTVAAPSVFIFPP |
| | | SDEQLKSGTASVVCLLNNFYPREA |
| | | KVQWKVDNALQSGNSQESVTEQD |
| | | SKDSTYSLSSTLTLSKADYEKHKV |
| | | YACEVTHQGLSSPVTKSFNRGEC |
| 282 | CX-188 HC | EVQLVESGGGLVQPGGSLRLSCAA |
| | | SGFTFSGYAMSWVRQAPGKGLEW |
| | | VAYISNSGGNAHYADSVKGRFTIS |
| | | RDNSKNTLYLQMNSLRAEDTAVY |
| | | YCTREDYGTSPFVYWGQGTLVTV |
| | | SSASTKGPSVFPLAPCSRSTSESTA |
| | | ALGCLVKDYFPEPVTVSWNSGAL |
| | | TSGVHTFPAVLQSSGLYSLSSVVT |
| | | VPSSSLGTKTYTCNVDHKPSNTKV |
| | | DKRVESKYGPPCPPCPAPEFLGGPS |
| | | VFLFPPKPKDTLMISRTPEVTCVVV |
| | | DVSQEDPEVQFNWYVDGVEVHN |
| | | AKTKPREEQFNSTYRVVSVLTVLH |
| | | QDWLNGKEYKCKVSNKGLPSSIE |
| | | KTISKAKGQPREPQVYTLPPSQEE |
| | | MTKNQVSLTCLVKGFYPSDIAVE |
| | | WESNGQPENNYKTTPPVLDSDGSF |
| | | FLYSRLTVDKSRWQEGNVFSCSV |
| | | MHEALHNHYTQKSLSLSLG |
| 283 | VL CDRL1-PDL1 | RASQSISSYLN |
| 284 | CDRL2 | AASSLQS |
| 285 | CDRL3 | DNGYPST |
| 286 | CDRH1 | SYAMS |
| 287 | CDRH2 | SSIWRNGIVTVYADS |
| 288 | CDRH3 | WSAAFDY |
| 289 | MM-PDL1 | YCEVSELFVLPWCMG |
| 290 | MM | SCLMHPHYAHDYCYV |
| 291 | MM | LCEVLMLLQHPWCMG |
| 292 | MM | IACRHFMEQLPFCHH |
| 293 | MM | FGPRCGEASTCVPYE |
| 294 | MM | ILYCDSWGAGCLTRP |
| 295 | MM | GIALCPSHFCQLPQT |
| 296 | MM | DGPRCFVSGECSPIG |
| 297 | MM | LCYKLDYDDRSYCHI |
| 298 | MM | PCHPHPYDARPYCNV |
| 299 | MM | PCYWHPFFAYRYCNT |
| 300 | MM | VCYYMDWLGRNWCSS |
| 301 | MM | LCDLFKLREFPYCMG |
| 302 | MM | YLPCHFVPIGACNNK |
| 303 | MM | IFCHMGVVVPQCANY |
| 304 | MM | ACHPHPYDARPYCNV |
| 305 | MM | PCHPAPYDARPYCNV |
| 306 | MM | PCHPHAYDARPYCNV |
| 307 | MM | PCHPHPADARPYCNV |
| 308 | MM | PCHPHPYAARPYCNV |
| 309 | MM | PCHPHPYDAAPYCNV |
| 310 | MM | PCHPHPYDARPACNV |
| 311 | MM | PCHPHPYDARPYCAV |
| 312 | MM | PCHAHPYDARPYCNV |
| 313 | MM | PCHPHPYDARAYCNV |
| 314 | Light Chain CX- | QGQSGSGIALCPSHFCQLPQTGGG |
| | 072 | SSGGSGGSGGISSGLLSGRSDNHG |
| | | GSDIQMTQSPSSLSASVGDRVTITC |
| | | RASQSISSYLNWYQQKPGKAPKLL |
| | | IYAASSLQSGVPSRFSGSGSGTDFT |
| | | LTISSLQPEDFATYYCQQDNGYPST |
| | | FGGGTKVEIKRTVAAPSVFIFPPSD |
| | | EQLKSGTASVVCLLNNFYPREAKV |
| | | QWKVDNALQSGNSQESVTEQDSK |
| | | DSTYSLSSTLTLSKADYEKHKVYA |
| | | CEVTHQGLSSPVTKSFNRGEC |
| 315 | Heavy Chain CX- | EVQLLESGGGLVQPGGSLRLSCAA |
| | 072 (+IgG4 | SGFTFSSYAMSWVRQAPGKGLEW |
| | S228P) | VSSIWRNGIVTVYADSVKGRFTISR |
| | | DNSKNTLYLQMNSLRAEDTAVYY |
| | | CAKWSAAFDYWGQGTLVTVSSAS |
| | | TKGPSVFPLAPCSRSTSESTAALGC |
| | | LVKDYFPEPVTVSWNSGALTSGV |
| | | HTFPAVLQSSGLYSLSSVVTVPSSS |
| | | LGTKTYTCNVDHKPSNTKVDKRV |
| | | ESKYGPPCPPCPAPEFLGGPSVFLF |
| | | PPKPKDTLMISRTPEVTCVVVDVS |
| | | QEDPEVQFNWYVDGVEVHNAKT |
| | | KPREEQFNSTYRVVSVLTVLHQD |
| | | WLNGKEYKCKVSNKGLPSSIEKTI |
| | | SKAKGQPREPQVYTLPPSQEEMTK |
| | | NQVSLTCLVKGFYPSDIAVEWESN |
| | | GQPENNYKTTPPVLDSDGSFFLYS |
| | | RLTVDKSRWQEGNVFSCSVMHEA |
| | | LHNHYTQKSLSLSLG |

### ASPECTS OF THE INVENTION

The following are non-limiting aspects of the invention described herein:

1. A process for producing a purified composition of intact activatable antibody, the process comprising:

(a) loading an aqueous feedstock comprising water, an intact activatable antibody, a clipped impurity, and a first salt onto a chromatography column,
wherein the chromatography column comprises a stationary phase that comprises a support matrix and ligands bound thereto,
   wherein the ligands comprise a hydrophobic substituent, and
wherein the intact activatable antibody comprises (i) at least a first antigen binding domain (AB) that has a specific binding affinity for a first biological target, and (ii) a first prodomain,
wherein the at least first AB comprises a first antibody light variable domain (VL) and a first antibody heavy variable domain (VH),
wherein the first prodomain comprises a first masking moiety (MM) and a first cleavable moiety (CM), and
wherein the first AB is coupled to the first prodomain; and
(b) eluting the chromatography column with an eluent comprising water and a second salt to generate an eluate that comprises a purified composition comprising intact activatable antibody,
wherein the eluate is substantially depleted of the clipped impurity.

2. The process of aspect 1, wherein the elution step (b) is carried out under isocratic conditions.

3. The process of any of aspects 1-2, wherein after step (b), the process further comprises a column cleaning step that comprises washing the chromatography column with a cleaning agent, and wherein the process does not comprise a step of eluting bound clipped impurity prior to the cleaning step.

4. The process of any of aspects 1-3, wherein the first salt and the second salt each independently comprise an anion selected from the group consisting of PO₄³⁻, SO₄²⁻, OH⁻, HPO₄²⁻, CH₃COO⁻, citrate ion, an amino acid anion, F⁻, Cl⁻, Br⁻, H₂PO₄⁻, I⁻, NO₃⁻, ClO₄⁻, and SCN⁻.

5. The process of any of aspects 1-4, wherein the first and second salts each independently comprise a cation selected from the group consisting of N(CH₃)₄⁺, NH₄⁺, Cs⁺, Rb⁺, K⁺, Na⁺, H⁺, Ca⁺, Mg²⁺, Al₃₊, and an amino acid cation.

6. The process of any of aspects 1-5, wherein the ligands comprise one or more hydrophobic substituents selected from the group consisting of a straight chain alkyl substituent, a branched alkyl substituent, and an aryl substituent.

7. The process of any of aspects 1-6, wherein the ligands comprise a C₄ to C₁₀ alkyl substituent.

8. The process of any of aspects 1-7, wherein the ligands comprise a branched alkyl substituent.

9. The process of any of aspects 1-8, wherein the ligands comprise an aryl substituent.

10. The process of aspect 9, wherein the aryl substituent is phenyl.

11. The process of any of aspects 1-10, wherein the stationary phase is a hydrophobic interaction chromatography (HIC) stationary phase.

12. The process of aspect 11, wherein the first salt and the second salt each independently comprise an anion selected from the group consisting of PO₄³⁻, SO₄²⁻, OH⁻, HPO₄²⁻, F⁻, CH₃COO⁻, citrate ion, an amino acid anion, and Cl⁻.

13. The process of any of aspects 11-12, wherein the first salt and second salt each independently comprise an anion selected from the group consisting of PO₄³⁻, SO₄²⁻, and HPO₄²⁻.

14. The process of any of aspects 11-13, wherein the first and second salt each independently comprise a cation selected from the group consisting of N(CH₃)₄⁺, NH₄⁺, Cs⁺, Rb⁺, K⁺, Na⁺, H⁺, Ca⁺, Mg²⁺, Al³⁺, and an amino acid cation.

15. The process of any of aspects 11-14, wherein the first and second salt each independently comprise a cation selected from the group consisting of NH₄⁺, K⁺, Na⁺, Li⁺, and Mg²⁺.

16. The process of any of aspects 11-15, wherein the first and second salt each independently comprise a cation selected from the group consisting of NH₄⁺, K⁺, and Na⁺.

17. The process of any of aspects 11-16, wherein the first and second salt each independently comprise a cation selected from the group consisting of NH₄⁺, K⁺, and Na⁺ and an anion selected from the group consisting of PO₄³⁻, SO₄²⁻, OH⁻, HPO₄²⁻, CH₃COO—, citrate ion, F, Cl⁻, Br⁻, H₂PO₄⁻, I⁻, NO₃⁻, ClO₄⁻, and SCN⁻.

18. The process of any of aspects 11-17, wherein the first and second salt are each independently selected from the group consisting of (NH₄)₂SO₄, Na₂SO₄, Na₃PO₄, K₃PO₄. NaCl, KCl, and CH₃COONH₄.

19. The process of any of aspects 11-18, wherein the first and second salt are each independently selected from the group consisting of (NH₄)₂SO₄, Na₂SO₄, Na₃PO₄, and K₃PO₄.

20. The process of any of aspects 11-19, wherein the first and second salt are each independently selected from the group consisting of (NH₄)₂SO₄ and Na₂SO₄,

21. The process of any of aspects 11-20, wherein the first salt and the second salt are the same salt.

22 The process of any of aspects 11-20, wherein the first salt and the second salt are different salts.

23 The process of any of aspects 11-22, wherein the aqueous feedstock and eluent comprise a first salt concentration and a second salt concentration, respectively, and wherein the first salt concentration is greater than the second salt concentration.

24. The process of any of aspects 1-10, wherein the stationary phase is a multimodal chromatography (MMC) stationary phase, whereby the ligands further comprise at least one further substituent that facilitates separation on the basis of an interaction other than hydrophobicity.

25. The process of aspect 24, wherein the at least one further substituent facilitates separation on the basis of an interaction selected from the group consisting of electrostatic, hydrogen bonding, and thiophilicity.

26. The process of any of aspects 24-25, wherein the at least one further substituent is selected from the group consisting of a sulfide substituent, a carboxyl substituent, and an amine substituent.

27. The process of any of aspects 24-26, wherein the at least one further substituent comprises a carboxyl substituent.

28. The process of any of aspects 24-27, wherein the at least one further substituent comprises an amine substituent.

29. The process of any of aspects 24-28, wherein the at least one further substituent comprises a sulfide substituent.

30. The process of any of aspects 24-29, wherein the first salt and the second salt each independently comprise an anion selected from the group consisting of Cl⁻, Br⁻, H₂PO₄⁻, I⁻, NO₃⁻, an amino acid anion, ClO₄⁻, and SCN⁻.

31. The process of any of aspects 24-30, wherein the first and second salt each independently comprise a cation selected from the group consisting of N(CH₃)₄⁺, NH₄⁺, Ba⁺, Ca²⁺, Mg²⁺, Cs⁺, Rb⁺, K⁺, Na⁺, and an amino acid cation.

32. The process of any of aspects 24-31, wherein at least one of the first and second salt is an amino acid cation.

33. The process of aspect 32, wherein the amino acid cation is an arginine cation.

34. The process of any of aspects 24-33, wherein the first salt and the second salt are the same.

35. The process of any of aspects 24-33, wherein the first salt and the second salt are different.

36. The process of any of aspects 24-31, wherein the first salt is comprises an Na+ cation and the second salt comprises both an Na+ cation and an arginine cation.

37. The process of any of aspects 24-36, wherein the aqueous feedstock and eluate comprise a first salt concentration and a second salt concentration, respectively, and wherein the second salt concentration is greater than the first salt concentration.

38. The process of aspect 37, wherein the second salt concentration is at least 2 times greater than the first salt concentration.

39. The process of aspect 37, wherein the second salt concentration is at least 3 times greater than the first salt concentration.

40. The process of aspect 36 or 37, wherein the concentration of Na+ cation in the second salt is the same as the concentration of Na+ cation in the first salt, and wherein the second salt further comprises an arginine cation not present in the first salt.

41. The process of any of aspects 11-20, wherein the first and second salt are the same, and are selected from the group consisting of (NH₄)₂SO₄ and Na₂SO₄.

42. The process of aspect 23 or 41, wherein the first salt concentration is at least 2 times greater than the second salt concentration.

43. The process of aspect 23 or 41, wherein the first salt concentration is at least 3 times greater than the second salt concentration.

44. The process of any of aspects 1-43, wherein the aqueous feedstock comprises a pH in the range of from about 5.0 to about 8.0, or about 5.0 to about 7.5, or from about 5.0 to about 7.0, or from about 5.5 to about 6.5.

45. The process of any of aspects 1-44, wherein the pH of the aqueous feedstock is greater than the pH of the eluent.

46. The process of any of aspects 1-44, wherein the pH of the aqueous feedstock is lower than the pH of the eluent.

47. The process of any of aspects 1-44, wherein the pH of the aqueous feedstock is about the same as the pH of the eluent.

48. The process of aspect 47, wherein the pH of the the aqueous feedstock and the pH of the eluent is about 5.5 to about 6.5.

49. The process of aspect 47, wherein the pH of the the aqueous feedstock and the pH of the eluent is about 5.8 to about 6.2.

50. The process of aspect 11, wherein the first and second salt are individually selected from the group consisting of (NH₄)₂SO₄ and Na₂SO₄, and wherein the pH of the the aqueous feedstock and the pH of the eluent is about 5.5 to about 6.5.

51. The process of aspect 50, wherein the first salt concentration is about 1.0 to 2.0 M, and wherein the second salt concentration is about 0.2 to about 0.6 M.

52. The process of aspect 50, wherein the first salt concentration is at least two times, at least three times greater, at least four times greater, or at least five times greater than the second salt concentration.

53. The process of aspect 24, wherein the first salt is NaCl and the second salt is NaCl and arginine HCl, and wherein the pH of the the aqueous feedstock and the pH of the eluent is about 5.5 to about 6.5.

54. The process of aspect 53, wherein the first salt concentration is about 30 mM NaCl and the second salt concentration is about 30 mM NaCl and about 90 mM arginine HCl.

55. The process of aspect 53, wherein the second salt concentration is at least two times, at least three times greater, at least four times greater, or at least five times greater than the first salt concentration.

56 The process of any of aspects 1-55, wherein steps (a) and (b) are carried out at a temperature in the range of from about 3° C. to about 40° C., or in the range of from about 10° C. to about 30° C., or in the range of from about 15° C. to about 30° C., or in the range of about 22° C.+/−4° C.

57. The process of aspect 56, wherein steps (a) and (b) are carried about at a temperature of 22° C.+/−4° C.

58. The process of any of aspects 1-57, wherein the process results in a total protein yield of at least about 70%, or at least about 71%, or at least about 72%, or at least about 73%, or at least about 74%, or at least about 75%, or at least about 76%, or at last about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, as determined by absorbance at a wavelength of 280 nm.

59. The process of any of aspects 1-58, wherein the ratio of quantity of clipped impurity in the aqueous feedstock to quantity of clipped impurity in the eluate on a percent clipped impurity basis is at least about 2, or at least about 3, or at least about 4, or at least about 5, or at least about 6, or at least about 7, or at least about 8, or at least about 9, or at least about 10, or at least about 15, or at least about 20, as determined by reducing SDS-cGE.

60. The process of any of aspects 1-59, wherein the eluate comprises less than about less than about 15%, or less than about 14%, or less than about 13%, or less than about 12%, or less than about 11%, or less than about 10%, or less than about 10%, or less than about 9%, or less than about 8%, or less than about 7%, or less than about 6% of the relative quantity of clipped impurity present in the aqueous feedstock, as determined by reducing SDS-cGE.

61. The process of aspect 60, wherein the eluate comprises less than about 5%, or less than about 4%, or less than about 3%, or less than about 2%, or less than about 1%, or less than about 0.9%, or less than about 0.8%, or less than about 0.7%, or less than about 0.6%, or less than about 0.5% clipped impurity, as determined by reducing SDS-cGE.

62 The process of aspect 61, wherein the eluate comprises less than about 2%, or less than about 1%, or less than about 0.9%, or less than about 0.8%, or less than about 0.7%, or less than about 0.6%, or less than about 0.5% clipped impurity, as determined by reducing SDS-cGE.

63. The process of any of aspects 1-62, wherein the eluate comprises a relative quantity of clipped impurity in the range of from about 0.1% to about 15% clipped impurity, or from about 0.1% to about 10% clipped impurity, or from about 0.1% to about 10% clipped impurity, or from about 0.1% to about 5% clipped impurity, or from about 0.1% to about 4% clipped impurity, or from about 0.1% to about 3% clipped impurity, or from about 0.1% to about 2% clipped impurity, or from about 0.1% to about 1% clipped impurity, as determined by reducing SDS-cGE.

64. The process of any of aspects 1-62, wherein the eluate comprises no detectable clipped impurity, as determined by reducing SDS-cGE.

65. The process of any of aspects 1-64, wherein the aqueous feedstock and the eluate each independently comprises a buffer selected from the group consisting of MES, MOPS, or HEPES.

66. The process of any of aspects 59-64, wherein the first AB comprises an antibody light chain and an antibody heavy chain, and wherein first prodomain is coupled to the antibody light chain of the first AB via a peptide bond, and wherein the relative quantity of clipped impurity is determined based on the light chain clipped variant and light chain of the intact activatable antibody as determined by reducing SDS-cGE.

67 The process of any of aspects 1-64, wherein the first prodomain is coupled to the first AB via a peptide bond.

68. The process of any of aspects 1-67, wherein the aqueous feedstock further comprises an impurity selected from the group consisting of host cell protein (HCP) and high molecular weight species (HMWS).

69. The process of aspect 68, wherein the aqueous feedstock further comprises HCP.

70. The process of aspect 69, wherein the ratio of the quantity of HCP in the aqueous feedstock to the quantity of HCP in the eluate on a ppm basis is at least about 2, or at least about 3, or at least about 4, or at least about 5, or at least about 6, or at least about 7, or at least about 8, or at least about 9, or at least about 10, as determined by a corresponding HCP ELISA assay.

71. The process of any of aspects 69-70, wherein the eluate comprises less than about 150 ppm, or less than about 140 ppm, or less than about 130 ppm, or less than about 120 ppm, or less than about 110 ppm, or less than about 100 ppm, or less than about 90 ppm, or less than about 80 ppm, or less than about 70 ppm, or less than about 60 ppm, or less than about 50 ppm, or less than about 45 ppm, or less than about 40 ppm, or less than about 35 ppm, or less than about 30 ppm, or less than about 25 ppm, or less than about 20 ppm, or less than about 15 ppm, or less than about 10 ppm HCP, or less than about 5 ppm HCP, or less than about 1 ppm HCP, as measured by a corresponding HCP ELISA.

72. The process of any of aspects 69-70, wherein the eluate comprises a quantity of HCP in the range of from about 0.5 ppm HCP to about 150 ppm HCP, or from about 0.5 ppm HCP to about 140 ppm HCP, or 0.5 ppm HCP to about 130 ppm HCP, or from about 0.5 ppm HCP to about 120 ppm, or from about 0.5 ppm HCP to about 110 ppm, or from about 0.5 ppm HCP to about 100 ppm HCP, or from about 0.5 ppm HCP to about 90 ppm HCP, or from about 0.5 ppm HCP to about 90 ppm HCP, or from about 0.5 ppm HCP to about 80 ppm HCP, or from about 0.5 ppm HCP to about 70 ppm HCP, or from about 0.5 ppm HCP to about 60 ppm HCP, or from about 0.5 ppm to about 50 ppm HCP, or from about 0.5 ppm HCP to about 45 ppm HCP, or from about 0.5 ppm HCP to about 40 ppm HCP, or from about 0.5 ppm HCP to about 40 ppm HCP, or from about 0.5 ppm HCP to about 35 ppm HCP, or from about 0.5 ppm HCP to about 30 ppm HCP, or from about 1 ppm HCP to about 25 ppm HCP, or from about 1 ppm HCP to about 20 ppm HCP, or from about 0.5 ppm HCP to about 15 ppm HCP, or from about 0.5 ppm HCP to about 10 ppm HCP.

73. The process of any of aspects 69-70, wherein the eluate comprises no detectable HCP, as measured by a corresponding HCP ELISA assay.

74. The process of any of aspects 1-73, wherein the aqueous feedstock further comprises HMWS.

75. The process of aspect 74, wherein the ratio of the quantity of HMWS in the aqueous feedstock to the quantity of HMWS in the eluate on a percent peak area basis is at least about 2, or at least about 3, or at least about 4, or at least about 5, as measured by Size Exclusion (SE)-HPLC.

76. The process of any of aspects 74-75, wherein the eluate comprises less than about 5% HMWS, or less than about 4% HMWS, or less than about 3% HMWS, or less than about 2% HMWS, or less than about 1% HMWS, as measured by SE-HPLC.

77. The process of any of aspects 74-75, wherein the eluate comprises a quantity of HMWS in the range of from about 0.2% HMWS to about 5% HMWS, or from about 0.2% HMWS to about 4% HMWS, or from about 0.2% HMWS to about 3% HMWS, or from about 0.2% HMWS to about 2% HMWS, or from about 0.2% HMWS to about 1% HMWS, as determined by SE-HPLC.

78. The process of any of aspects 74-75, wherein the eluate comprises no detectable HMWS, as measured by SE-HPLC.

79. The process of any of aspects 1-78, wherein the eluate comprises at least about 90% intact activatable antibody, or at least about 91%, or at least about 92%, or at least about 93%, or at least about 94%, or at least about 95% intact activatable antibody, as determined by reducing SDS-cGE.

80. The process of aspect 79, wherein the first AB comprises an antibody light chain and an antibody heavy chain, and wherein first prodomain is coupled to the antibody light chain of the first AB via a peptide bond, and wherein the percent intact activatable antibody is determined based on the light chain of the intact activatable antibody and light chain clipped variant as determined by reducing SDS-cGE.

81. The process of any of aspects 1-80, wherein prior to step (a), the process further comprises:

(a⁰) subjecting a bioharvest composition comprising the intact activatable antibody and the clipped impurity to one or more intervening unit operations selected from the group consisting of a centrifugation step, a filtration step, an affinity chromatography step, a virus inactivation step, a size exclusion chromatography step, a virus filtration step, an ion exchange chromatography step, and a combination of any two or more thereof, to produce one or more bulk intermediate product compositions, wherein the aqueous feedstock comprises at least one bulk intermediate product composition.

82. The process of any of aspects 1-81, wherein the eluate is subjected to one or more downstream unit operations to generate a downstream product composition.

83 The process of aspect 82, wherein the one or more downstream unit operations is selected from the group consisting of a further purification process, a chemical synthesis process, a dilution process, a solvent exchange process, a formulating process, a lyophilization process, and any combination of two or more thereof.

84. The process of aspect 82 or 83, wherein the one or more downstream unit operations comprises a further purification process selected from the group consisting of a centrifugation step, a filtration step, an affinity chromatography step, a virus inactivation step, a size exclusion chromatography step, a virus filtration step, an ion exchange chromatography step, and any combination of two or more thereof.

85. The process of any of aspects 82-84, wherein the one or more downstream unit operations comprises a lyophilization process.

86. The process of any of aspects 82-85, wherein the one or more downstream unit operations is a chemical synthesis process.

87 The process of aspect 86, wherein the chemical synthesis process is a chemical conjugation reaction.

88. The process of any of aspects 1-87, wherein the aqueous feedstock comprises greater than about 0.5% clipped impurity, or greater than about 0.6%, or greater than about 0.7%, or greater than about 0.8%, or greater than about 0.9%, or greater than about 1%, or greater than about 1.5%, or greater than about 2%, or greater than about 2.5%, or greater than about 3%, or greater than about 3.5%, or greater than about 4%, or greater than about 4.5% clipped impurity, as determined by reducing SDS-cGE.

89. The process of any of aspects 1-88, wherein the intact activatable antibody comprises a second AB and a second prodomain, and wherein the clipped impurity comprises single-arm clipped impurity, as determined by mass spectrometry.

90 The process of aspect 89, wherein the second AB is the same as the first AB, and wherein the second prodomain is the same as the first prodomain.

91. The process of aspect 89 or 90, wherein the clipped impurity consists essentially of single-arm clipped impurity, as determined by mass spectrometry.

92. The process of aspect 89 or 90, wherein the clipped impurity consists of single-arm clipped impurity, as determined by mass spectrometry.

93. A purified intact activatable antibody composition produced by the process of any of aspects 1-92.

94. A purified product composition comprising the downstream product composition of any of aspects 82-87.

95. The process of any of aspects 1-92, wherein the eluate comprises at least about 90% intact activatable antibody, as determined by reducing SDS-cGE, less than about 15% clipped impurity, as determined by reducing SDS-cGE, less than about 5% HMWS, as determined by SE-HPLC, and less than about 150 ppm HCP, as determined by a corresponding HCP ELISA.

96. The process of aspect 95, wherein the eluate comprises less than about 5% clipped impurity, as determined by reducing SDS-cGE.

97. The process of aspect 96, wherein the eluate comprises less than about 3% clipped impurity, as determined by reducing SDS-cGE.

98. A purified intact activatable antibody composition comprising the eluate produced by the process of any of aspects 1-97.

99. A purified intact activatable antibody composition comprising at least about 90% intact activatable antibody, as determined by reducing SDS-cGE, less than about 15% clipped impurity, as determined by reducing SDS-cGE, less than about 5% HMWS, as determined by SE-HPLC, and less than about 150 ppm HCP, as determined by a corresponding HCP ELISA.

100. The purified intact activatable antibody composition of aspect 98 or 99, wherein the composition comprises less than about 14% clipped impurity, or less than about 13% clipped impurity, or less than about 12% clipped impurity, or less than about 12% clipped impurity, or less than about 11% clipped impurity, or less than about 10% clipped impurity, or less than about 9% clipped impurity, or less than about 8% clipped impurity, or less than about 7% clipped impurity, or less than about 6% clipped impurity, as determined by reducing SDS-cGE.

101. The purified intact activatable antibody composition of any of aspects 98-100, wherein the composition comprises less than about 5% clipped impurity, or less than about 4% clipped impurity, or less than about 3% clipped impurity, or less than about 2% clipped impurity, as determined by reducing SDS-cGE.

102. The purified intact activatable antibody composition of any of aspects 98-101, wherein the composition comprises less than about 1% clipped impurity, or less than about 0.9% clipped impurity, or less than about 0.8% clipped impurity, or less than about 0.7% clipped impurity, or less than about 0.6% clipped impurity, or less than about 0.5% clipped impurity, as determined by reducing SDS-cGE.

103. The purified intact activatable antibody composition of aspect 98 or 99, wherein the composition comprises a relative quantity of clipped impurity in the range of from about 0.1% to about 15% clipped impurity, or from about 0.1% to about 10% clipped impurity, or from about 0.1% to about 10% clipped impurity, or from about 0.1% to about 5% clipped impurity, or from about 0.1% to about 4% clipped impurity, or from about 0.1% to about 3% clipped impurity, or from about 0.1% to about 2% clipped impurity, or from about 0.1% to about 1% clipped impurity, as determined by reducing SDS-cGE.

104. The purified intact activatable antibody composition of aspect 98 or 99, wherein the composition comprises no detectable clipped impurity, as determined by reducing SDS-cGE.

105. The purified intact activatable antibody composition of any of aspects 98-104, wherein the composition comprises less than about 140 ppm HCP, or less than about 130 ppm HCP, or less than about 120 ppm HCP, or less than about 110 ppm HCP, or less than about 100 ppm HCP, or less than about 90 ppm HCP, or less than about 80 ppm HCP, or less than about 70 ppm HCP, or less than about 60 ppm HCP, or less than about 50 ppm HCP, or less than about 45 ppm HCP, or less than about 40 ppm HCP, or less than about 35 ppm HCP, or less than about 30 ppm HCP, or less than about 25 ppm HCP, or less than about 20 ppm HCP, or less than about 15 ppm HCP, or less than about 10 ppm HCP, as determined by a corresponding HCP ELISA assay.

106. The purified intact activatable antibody composition of any of aspects 98-105, wherein the composition comprises a quantity of HCP in the range of from about 1 ppm HCP to about 150 ppm HCP, or from about 1 ppm HCP to about 140 ppm HCP, or 1 ppm HCP to about 130 ppm HCP, or from about 1 ppm HCP to about 120 ppm, or from about 1 ppm HCP to about 110 ppm, or from about 1 ppm HCP to about 100 ppm HCP, or from about 1 ppm HCP to about 90 ppm HCP, or from about 1 ppm HCP to about 90 ppm HCP, or from about 1 ppm HCP to about 80 ppm HCP, or from about 1 ppm HCP to about 70 ppm HCP, or from about 1 ppm HCP to about 60 ppm HCP, or from about 1 ppm to about 50 ppm HCP, or from about 1 ppm HCP to about 45 ppm HCP, or from about 1 ppm HCP to about 40 ppm HCP, or from about 1 ppm HCP to about 40 ppm HCP, or from about 1 ppm HCP to about 35 ppm HCP, or from about 1 ppm HCP to about 30 ppm HCP, or from about 1 ppm HCP to about 25 ppm HCP, or from about 1 ppm HCP to about 20 ppm HCP, or from about 1 ppm HCP to about 15 ppm HCP, or from about 1 ppm HCP to about 10 ppm HCP.

107. The purified intact activatable antibody composition of any of aspects 98-106, wherein the composition comprises less than about 4% HMWS, or less than about 3% HMWS, or less than about 2% HMWS, or less than about 1% HMWS, as determined by SE-HPLC.

108. The purified intact activatable antibody composition of aspect 98 or 99, wherein the composition comprises a quantity of HMWS in the range of from about 0.2% HMWS to about 5% HMWS, or from about 0.2% HMWS to about 4% HMWS, or from about 0.2% HMWS to about 3% HMWS, or from about 0.2% HMWS to about 2% HMWS, or from about 0.2% HMWS to about 1% HMWS, as determined by SE-HPLC

109. The purified intact activatable antibody composition of any of aspects 98-108, wherein the composition comprises at least about 91%, or at least about 92%, or at least about 93%, or at least about 94%, or at least about 95% intact activatable antibody, as determined by reducing SDS-cGE.

110. The purified intact activatable antibody composition of any of aspects 98-109, wherein the composition is lyophilized.

111. A pharmaceutically acceptable composition comprising the composition of any of aspects 98-110 and a pharmaceutically acceptable excipient.

112. The process of any of aspects 1-92, or 95-97, the purified intact activatable antibody composition of any of aspects 93, or 98-110, the purified product composition of aspect 94, or the pharmaceutically acceptable composition of aspect 111, wherein the first CM in the intact activatable antibody comprises a substrate for a protease that is over-expressed in a diseased tissue relative to healthy tissue.

113. The process of any of aspects 1-92, 95-97, or 112, the purified intact activatable antibody composition of any of aspects 93, 98-110, the purified product composition of any of aspects 94 or 112, or the pharmaceutically acceptable composition of any of aspects 110 or 111, wherein the intact activatable antibody further comprises (iii) a second AB that has a specific binding affinity for a second biological target, and (iv) a second prodomain,
wherein the second AB comprises a second VL, and a second VH,
wherein the second prodomain comprises a second MM and a second CM, and
wherein the second AB is coupled to the second prodomain.

114. The process, purified intact activatable antibody composition, purified product composition, or pharmaceutically acceptable composition of aspect 113, wherein the second CM comprises a substrate for a protease that is overexpressed in a diseased tissue relative to healthy tissue.

115. The process, purified intact activatable antibody composition, purified product composition, or pharmaceutically acceptable composition of any of aspects 113-114, wherein the first CM and the second CM comprise identical amino acid sequences.

116. The process, purified intact activatable antibody composition, purified product composition, or pharmaceutically acceptable composition of any of aspects 113-115, wherein the first CM and the second CM comprise different amino acid sequences.

117. The process, purified intact activatable antibody composition, purified product composition, or pharmaceutically acceptable composition of any of aspects 113-116, wherein the first biological target and the second biological target are different.

118. The process, purified intact activatable antibody composition, purified product composition, or pharmaceutically acceptable composition of any of aspects 113-117, wherein at least one of the first biological target and the second biological target is a T cell co-receptor.

119. The process, purified intact activatable antibody composition, purified product composition, or pharmaceutically acceptable composition of aspect 118, wherein the T cell co-receptor is a cluster of differentiation 3 (CD3) T cell co-receptor.

120. The process, purified intact activatable antibody composition, purified product composition, or pharmaceutically acceptable composition of any of aspects 113-115, wherein the second AB is identical to the first AB, and wherein the second prodomain is identical to the first prodomain.

121. The process, purified intact activatable antibody composition, purified product composition, or pharmaceutically acceptable composition of aspect 120, wherein the first biological target and the second biological target are CD166.

122. The process, purified intact activatable antibody composition, purified product composition, or pharmaceutically acceptable composition of aspect 120, wherein the first biological target and the second biological target are PD-1.

123. The process, purified intact activatable antibody composition, purified product composition, or pharmaceutically acceptable composition of aspect 120, wherein the first biological target and the second biological target are PDL-1.

While the foregoing invention has been described in some detail for purposes of clarity and understanding, it will be clear to one skilled in the art from a reading of this disclosure that various changes in form and detail can be made without departing from the true scope of the invention. It is understood that the materials, examples, and embodiments described herein are for illustrative purposes only and not intended to be limiting and that various modifications or changes in light thereof will be suggested to persons skilled in the art and are to be included within the spirit and scope of the appended claims.

We claim:

1. A composition comprising an intact activatable antibody and a detectable clipped variant thereof,
wherein the intact activatable antibody comprises (i) at least a first antigen binding domain (AB) that has a specific binding affinity for a first biological target, and (ii) a first prodomain,
wherein the at least first AB comprises a first antibody light variable domain (VL) and a first antibody heavy variable domain (VH),
wherein the first prodomain comprises a first masking moiety (MM) and a first cleavable moiety (CM),
wherein the first AB is coupled to the first prodomain,
wherein the detectable clipped variant of the intact activatable antibody comprises the AB of the activatable antibody but lacks all or a portion of the MM,
wherein the composition includes at least 95% intact activatable antibody and 0.05 to 5% detectable clipped variant thereof, as determined by sodium dodecyl sulfate capillary gel electrophoresis (SDS-cGE), and
wherein the percentages of intact activatable antibody and the detectable clipped variant thereof total 100%.

2. The composition of claim 1, wherein the detectable clipped variant comprises an antigen binding domain (AB) and at least a portion of a cleavable moiety (CM).

3. The composition of claim 1, further comprising less than 150 ppm host cell proteins (HCP) as determined by a corresponding HCP ELISA and/or 0.1 to 5% high molecular weight species (HMWS) as determined by size exclusion high performance liquid chromatography (SE-HPLC).

4. The composition of claim 1, further comprising about 0.5 ppm to 100 ppm host cell proteins (HCP) as determined by a corresponding HCP ELISA and 0.1 to 3% HMWS as determined by SE-HPLC.

5. The composition of claim 1, comprising at least 97% intact activatable antibody, 0.05 to 3% detectable clipped variant, as determined by SDS-cGE, about 0.5 ppm to 100 host cell proteins (HCP) as determined by a corresponding HCP ELISA, and 0.1 to 3% HMWS as determined by SE-HPLC, wherein the percentages of intact activatable antibody and the detectable clipped variant thereof total 100%.

6. The composition of claim 1, comprising at least 97% of the intact activatable antibody, 0.05 to 3% detectable clipped variant as determined by SDS-cGE, and 0.1 to 3% HMWS as determined by SE-HPLC, wherein the percentages of intact activatable antibody and the detectable clipped variant thereof total 100%.

7. The composition of claim 1, wherein the composition is an aqueous composition further comprising an anion selected from Cl⁻, Br⁻, H₂PO₄⁻, I⁻, NO₃⁻, ClO₄⁻ and SCN⁻, a cation selected from N(CH₃)₄⁺, NH₄⁺, Ba⁺, Ca²⁺, Mg²⁺, Cs⁺, Rb⁺, K⁺, Na⁺, or a combination of the anion and the cation.

8. The composition of claim 7, comprising 10 mM to 100 mM of the anion, cation, MES, MOPS, HEPES, (NH₄)₂SO₄, Na₂SO₄, Na₃PO₄, K₃PO₄, NaCl, KCl, and CH₃COONH₄, or combination thereof.

9. The composition of claim 1, further comprising arginine, tryptophan, asparagine, glutamine, lysine, histidine, serine, proline, or a salt thereof.

10. The composition of claim 9, comprising the arginine, tryptophan, asparagine, glutamine, lysine, histidine, serine, proline, or salt thereof at a concentration of 10 mM to 150 mM.

11. The composition of claim 1, wherein the composition is an aqueous composition having a pH of about 5.0 to about 8.0, or about 5.0 to about 7.5, or about 5.0 to about 7.0, or about 5.5 to about 6.5.

12. The composition of claim 1, wherein the composition is an aqueous composition having a conductivity of from 110 to 150 mS/cm, 115 to 145 mS/cm, 120 to 140 mS/cm, 125 to 135 mS/cm or about 130 mS/cm.

13. The composition of claim 1, wherein the composition is free of buffering agents.

14. The composition of claim 1, wherein the composition has a pH outside of the buffering capacity of a buffering agent present in the composition.

15. The composition of claim 1, comprising a buffering agent having a pKa of 6.0 to 9.0.

16. A composition produced by:
(a) loading an aqueous feedstock comprising water, an intact activatable antibody, a detectable clipped impurity of the intact activatable antibody, and a first salt onto a chromatography column,
wherein the chromatography column comprises a stationary phase that comprises a support matrix and ligands bound thereto,
wherein the ligands comprise a hydrophobic substituent; and
(b) eluting the chromatography column with an eluent comprising water and a second salt to generate an eluate that comprises a purified composition comprising intact activatable antibody,
wherein the eluting step (b) is performed such that the eluate has a relative level of clipped impurity reduced by at least 20% compared to a level of clipped impurity in the aqueous feedstock in step (a),
wherein the intact activatable antibody comprises (i) at least a first antigen binding domain (AB) that has a specific binding affinity for a first biological target, and (ii) a first prodomain,
wherein the at least first AB comprises a first antibody light variable domain (VL) and a first antibody heavy variable domain (VH),
wherein the first prodomain comprises a first masking moiety (MM) and a first cleavable moiety (CM),
wherein the first AB is coupled to the first prodomain,
wherein the detectable clipped variant of the intact activatable antibody comprises the AB of the activatable antibody but lacks all or a portion of the MM.

17. An aqueous feedstock for a hydrophobic interaction chromatography (HIC) or multimodal chromatography (MMC) chromatography column, comprising water, intact activatable antibody, at least 2% clipped variant of the intact activatable antibody as determined by SDS-cGE, at least 10 ppm HCP as determined by a corresponding HCP ELISA, and at least 1% HMWS as determined by SE-HPLC,
wherein the intact activatable antibody comprises (i) at least a first antigen binding domain (AB) that has a specific binding affinity for a first biological target, and (ii) a first prodomain,
wherein the at least first AB comprises a first antibody light variable domain (VL) and a first antibody heavy variable domain (VH),
wherein the first prodomain comprises a first masking moiety (MM) and a first cleavable moiety (CM),
wherein the first AB is coupled to the first prodomain, and
wherein the clipped variant of the intact activatable antibody comprises the AB of the activatable antibody but lacks all or a portion of the MM.

18. The aqueous feedstock for an HIC or MMC chromatography column of claim 17, comprising at least 90% intact activatable antibody as determined by SDS-cGE, at least 2 to 15% of the clipped variant of the intact activatable antibody as determined by SDS-cGE, at least 10 ppm HCP as determined by ELISA, and 1 to 6% HMWS as determined by SE-HPLC.

19. An HIC eluate of the aqueous feedstock of claim 18, comprising at least 95% intact activatable antibody as determined by SDS-cGE, less than 2% of the clipped variant of the intact activatable antibody as determined by SDS-cGE, less than 5 ppm HCP as determined by ELISA, and less than 1% HMWS as determined by SE-HPLC, wherein the percentages of intact activatable antibody and the clipped variant total 100%.

20. An MMC eluate of the aqueous feedstock of claim 17, comprising at least 97% intact activatable antibody as determined by SDS-cGE, less than 1% of the clipped variant of the intact activatable antibody as determined by SDS-cGE, less than 6 ppm HCP as determined by ELISA, and less than 1% HMWS as determined by SE-HPLC, wherein the percentages of intact activatable antibody and the clipped variant total 100%.


SEQUENCE LISTING (315 sequences)

SEQ ID NO: 1 - protein, 8 aa, synthetic construct
  REGION 1..8: note = Synthetic
LSGRSDNH

SEQ ID NO: 2 - protein, 8 aa, synthetic construct
  REGION 1..8: note = Synthetic
TGRGPSWV

SEQ ID NO: 3 - protein, 8 aa, synthetic construct
  REGION 1..8: note = Synthetic
PLTGRSGG

SEQ ID NO: 4 - protein, 8 aa, synthetic construct
  REGION 1..8: note = Synthetic
TARGPSFK

SEQ ID NO: 5 - protein, 12 aa, synthetic construct
  REGION 1..12: note = Synthetic
NTLSGRSENHSG

SEQ ID NO: 6 - protein, 12 aa, synthetic construct
  REGION 1..12: note = Synthetic
NTLSGRSGNHGS

SEQ ID NO: 7 - protein, 12 aa, synthetic construct
  REGION 1..12: note = Synthetic
TSTSGRSANPRG

SEQ ID NO: 8 - protein, 8 aa, synthetic construct
  REGION 1..8: note = Synthetic
TSGRSANP

SEQ ID NO: 9 - protein, 10 aa, synthetic construct
  REGION 1..10: note = Synthetic
VHMPLGFLGP

SEQ ID NO: 10 - protein, 8 aa, synthetic construct
  REGION 1..8: note = Synthetic
AVGLLAPP

SEQ ID NO: 11 - protein, 8 aa, synthetic construct
  REGION 1..8: note = Synthetic
AQNLLGMV

SEQ ID NO: 12 - protein, 8 aa, synthetic construct
  REGION 1..8: note = Synthetic
QNQALRMA

SEQ ID NO: 13 - protein, 8 aa, synthetic construct
  REGION 1..8: note = Synthetic
LAAPLGLL

SEQ ID NO: 14 - protein, 8 aa, synthetic construct
  REGION 1..8: note = Synthetic
STFPFGMF

SEQ ID NO: 15 - protein, 8 aa, synthetic construct
  REGION 1..8: note = Synthetic
ISSGLLSS

SEQ ID NO: 16 - protein, 8 aa, synthetic construct
  REGION 1..8: note = Synthetic
PAGLWLDP

SEQ ID NO: 17 - protein, 8 aa, synthetic construct
  REGION 1..8: note = Synthetic
VAGRSMRP

SEQ ID NO: 18 - protein, 8 aa, synthetic construct
  REGION 1..8: note = Synthetic
VVPEGRRS

SEQ ID NO: 19 - protein, 8 aa, synthetic construct
  REGION 1..8: note = Synthetic
ILPRSPAF

SEQ ID NO: 20 - protein, 8 aa, synthetic construct
  REGION 1..8: note = Synthetic
MVLGRSLL

SEQ ID NO: 21 - protein, 8 aa, synthetic construct
  REGION 1..8: note = Synthetic
QGRAITFI

SEQ ID NO: 22 - protein, 8 aa, synthetic construct
  REGION 1..8: note = Synthetic
SPRSIMLA

SEQ ID NO: 23 - protein, 8 aa, synthetic construct
  REGION 1..8: note = Synthetic
SMLRSMPL

SEQ ID NO: 24 - protein, 13 aa, synthetic construct
  REGION 1..13: note = Synthetic
ISSGLLSGRSDNH

SEQ ID NO: 25 - protein, 18 aa, synthetic construct
  REGION 1..18: note = Synthetic
AVGLLAPPGGLSGRSDNH

SEQ ID NO: 26 - protein, 22 aa, synthetic construct
  REGION 1..22: note = Synthetic
ISSGLLSSGGSGGSLSGRSDNH

SEQ ID NO: 27 - protein, 8 aa, synthetic construct
  REGION 1..8: note = Synthetic
LSGRSGNH

SEQ ID NO: 28 - protein, 9 aa, synthetic construct
  REGION 1..9: note = Synthetic
SGRSANPRG

SEQ ID NO: 29 - protein, 8 aa, synthetic construct
  REGION 1..8: note = Synthetic
LSGRSDDH

SEQ ID NO: 30 - protein, 8 aa, synthetic construct
  REGION 1..8: note = Synthetic
LSGRSDIH

SEQ ID NO: 31 - protein, 8 aa, synthetic construct
  REGION 1..8: note = Synthetic
LSGRSDQH

SEQ ID NO: 32 - protein, 8 aa, synthetic construct
  REGION 1..8: note = Synthetic
LSGRSDTH

SEQ ID NO: 33 - protein, 8 aa, synthetic construct
  REGION 1..8: note = Synthetic
LSGRSDYH

SEQ ID NO: 34 - protein, 8 aa, synthetic construct
  REGION 1..8: note = Synthetic
LSGRSDNP

SEQ ID NO: 35 - protein, 8 aa, synthetic construct
  REGION 1..8: note = Synthetic
LSGRSANP

SEQ ID NO: 36 - protein, 8 aa, synthetic construct
  REGION 1..8: note = Synthetic
LSGRSANI

SEQ ID NO: 37 - protein, 8 aa, synthetic construct
  REGION 1..8: note = Synthetic
LSGRSDNI

SEQ ID NO: 38 - protein, 8 aa, synthetic construct
  REGION 1..8: note = Synthetic
MIAPVAYR

SEQ ID NO: 39 - protein, 8 aa, synthetic construct
  REGION 1..8: note = Synthetic
RPSPMWAY

SEQ ID NO: 40 - protein, 8 aa, synthetic construct
  REGION 1..8: note = Synthetic
WATPRPMR

SEQ ID NO: 41 - protein, 8 aa, synthetic construct
  REGION 1..8: note = Synthetic
FRLLDWQW

SEQ ID NO: 42 - protein, 5 aa, synthetic construct
  REGION 1..5: note = Synthetic
ISSGL

SEQ ID NO: 43 - protein, 7 aa, synthetic construct
  REGION 1..7: note = Synthetic
ISSGLLS

SEQ ID NO: 44 - protein, 6 aa, synthetic construct
  REGION 1..6: note = Synthetic
ISSGLL

SEQ ID NO: 45 - protein, 15 aa, synthetic construct
  REGION 1..15: note = Synthetic
ISSGLLSGRSANPRG

SEQ ID NO: 46 - protein, 18 aa, synthetic construct
  REGION 1..18: note = Synthetic
AVGLLAPPTSGRSANPRG

SEQ ID NO: 47 - protein, 17 aa, synthetic construct
  REGION 1..17: note = Synthetic
AVGLLAPPSGRSANPRG

SEQ ID NO: 48 - protein, 13 aa, synthetic construct
  REGION 1..13: note = Synthetic
ISSGLLSGRSDDH

SEQ ID NO: 49 - protein, 13 aa, synthetic construct
  REGION 1..13: note = Synthetic
ISSGLLSGRSDIH

SEQ ID NO: 50 - protein, 13 aa, synthetic construct
  REGION 1..13: note = Synthetic
ISSGLLSGRSDQH

SEQ ID NO: 51 - protein, 13 aa, synthetic construct
  REGION 1..13: note = Synthetic
ISSGLLSGRSDTH

SEQ ID NO: 52 - protein, 13 aa, synthetic construct
  REGION 1..13: note = Synthetic
ISSGLLSGRSDYH

SEQ ID NO: 53 - protein, 13 aa, synthetic construct
  REGION 1..13: note = Synthetic
ISSGLLSGRSDNP

SEQ ID NO: 54 - protein, 13 aa, synthetic construct
  REGION 1..13: note = Synthetic
ISSGLLSGRSANP

SEQ ID NO: 55 - protein, 13 aa, synthetic construct
  REGION 1..13: note = Synthetic
ISSGLLSGRSANI

SEQ ID NO: 56 - protein, 18 aa, synthetic construct
  REGION 1..18: note = Synthetic
AVGLLAPPGGLSGRSDDH

SEQ ID NO: 57 - protein, 18 aa, synthetic construct
  REGION 1..18: note = Synthetic
AVGLLAPPGGLSGRSDIH

SEQ ID NO: 58 - protein, 18 aa, synthetic construct
  REGION 1..18: note = Synthetic
AVGLLAPPGGLSGRSDQH

SEQ ID NO: 59 - protein, 18 aa, synthetic construct
  REGION 1..18: note = Synthetic
AVGLLAPPGGLSGRSDTH

SEQ ID NO: 60 - protein, 18 aa, synthetic construct
  REGION 1..18: note = Synthetic
AVGLLAPPGGLSGRSDYH

SEQ ID NO: 61 - protein, 18 aa, synthetic construct
  REGION 1..18: note = Synthetic
AVGLLAPPGGLSGRSDNP

SEQ ID NO: 62 - protein, 18 aa, synthetic construct
  REGION 1..18: note = Synthetic
AVGLLAPPGGLSGRSANP

SEQ ID NO: 63 - protein, 18 aa, synthetic construct
  REGION 1..18: note = Synthetic
AVGLLAPPGGLSGRSANI

SEQ ID NO: 64 - protein, 13 aa, synthetic construct
  REGION 1..13: note = Synthetic
ISSGLLSGRSDNI

SEQ ID NO: 65 - protein, 18 aa, synthetic construct
  REGION 1..18: note = Synthetic
AVGLLAPPGGLSGRSDNI

SEQ ID NO: 66 - protein, 19 aa, synthetic construct
  REGION 1..19: note = Synthetic
GLSGRSDNHGGAVGLLAPP

SEQ ID NO: 67 - protein, 21 aa, synthetic construct
  REGION 1..21: note = Synthetic
GLSGRSDNHGGVHMPLGFLGP

SEQ ID NO: 68 - protein, 5 aa, synthetic construct
  REGION 1..5: note = Synthetic
GSGGS

SEQ ID NO: 69 - protein, 4 aa, synthetic construct
  REGION 1..4: note = Synthetic
GGGS

SEQ ID NO: 70 - protein, 4 aa, synthetic construct
  REGION 1..4: note = Synthetic
GGSG

SEQ ID NO: 71 - protein, 5 aa, synthetic construct
  REGION 1..5: note = Synthetic
GGSGG

SEQ ID NO: 72 - protein, 5 aa, synthetic construct
  REGION 1..5: note = Synthetic
GSGSG

SEQ ID NO: 73 - protein, 5 aa, synthetic construct
  REGION 1..5: note = Synthetic
GSGGG

SEQ ID NO: 74 - protein, 11 aa, synthetic construct
  REGION 1..11: note = Synthetic
GSSGGSGGSGG

SEQ ID NO: 75 - protein, 12 aa, synthetic construct
  REGION 1..12: note = Synthetic
GSSGGSGGSGGS

SEQ ID NO: 76 - protein, 16 aa, synthetic construct
  REGION 1..16: note = Synthetic
GSSGGSGGSGGSGGGS

SEQ ID NO: 77 - protein, 10 aa, synthetic construct
  REGION 1..10: note = Synthetic
GSSGGSGGSG

SEQ ID NO: 78 - protein, 11 aa, synthetic construct
  REGION 1..11: note = Synthetic
GSSGGSGGSGS

SEQ ID NO: 79 - protein, 4 aa, synthetic construct
  REGION 1..4: note = Synthetic
GGGS

SEQ ID NO: 80 - protein, 4 aa, synthetic construct
  REGION 1..4: note = Synthetic
GSSG

SEQ ID NO: 81 - protein, 13 aa, synthetic construct
  REGION 1..13: note = Synthetic
GGGSSGGSGGSGG

SEQ ID NO: 82 - protein, 5 aa, synthetic construct
  REGION 1..5: note = Synthetic
GGGSG

SEQ ID NO: 83 - protein, 5 aa, synthetic construct
  REGION 1..5: note = Synthetic
GSSGT

SEQ ID NO: 84 - protein, 6 aa, synthetic construct
  REGION 1..6: note = Synthetic
QGQSGS

SEQ ID NO: 85 - protein, 5 aa, synthetic construct
  REGION 1..5: note = Synthetic
GQSGS

SEQ ID NO: 86 - protein, 4 aa, synthetic construct
  REGION 1..4: note = Synthetic
QSGS

SEQ ID NO: 87 - protein, 7 aa, synthetic construct
  REGION 1..7: note = Synthetic
QGQSGQG

SEQ ID NO: 88 - protein, 6 aa, synthetic construct
  REGION 1..6: note = Synthetic
GQSGQG

SEQ ID NO: 89 - protein, 5 aa, synthetic construct
  REGION 1..5: note = Synthetic
QSGQG

SEQ ID NO: 90 - protein, 4 aa, synthetic construct
  REGION 1..4: note = Synthetic
SGQG

SEQ ID NO: 91 - protein, 6 aa, synthetic construct
  REGION 1..6: note = Synthetic
QGQSGQ

SEQ ID NO: 92 - protein, 5 aa, synthetic construct
  REGION 1..5: note = Synthetic
GQSGQ

SEQ ID NO: 93 - protein, 4 aa, synthetic construct
  REGION 1..4: note = Synthetic
QSGQ

SEQ ID NO: 94 - protein, 5 aa, synthetic construct
  REGION 1..5: note = Synthetic
QGQSG

SEQ ID NO: 95 - protein, 4 aa, synthetic construct
  REGION 1..4: note = Synthetic
QGQS

SEQ ID NO: 96 - protein, 16 aa, synthetic construct
  REGION 1..16: note = Synthetic
RSSKSLLHSNGITYLY

SEQ ID NO: 97 - protein, 7 aa, synthetic construct
  REGION 1..7: note = Synthetic
QMSNLAS

SEQ ID NO: 98 - protein, 9 aa, synthetic construct
  REGION 1..9: note = Synthetic
AQNLELPYT

SEQ ID NO: 99 - protein, 12 aa, synthetic construct
  REGION 1..12: note = Synthetic
GFSLSTYGMGVG

SEQ ID NO: 100 - protein, 9 aa, synthetic construct
  REGION 1..9: note = Synthetic
NIWWSEDKH

SEQ ID NO: 101 - protein, 11 aa, synthetic construct
  REGION 1..11: note = Synthetic
IDYGNDYAFTY

SEQ ID NO: 102 - protein, 15 aa, synthetic construct
  REGION 1..15: note = Synthetic
LCHPLVLSAWESCSS

SEQ ID NO: 103 - protein, 15 aa, synthetic construct
  REGION 1..15: note = Synthetic
LCHPAVLSAWESCSS

SEQ ID NO: 104 - protein, 15 aa, synthetic construct
  REGION 1..15: note = Synthetic
LCHPLVASAWESCSS

SEQ ID NO: 105 - protein, 15 aa, synthetic construct
  REGION 1..15: note = Synthetic
LEGWCLHPLCLWGAG

SEQ ID NO: 106 - protein, 15 aa, synthetic construct
  REGION 1..15: note = Synthetic
LCAPLVLSAWESCSS

SEQ ID NO: 107 - protein, 15 aa, synthetic construct
  REGION 1..15: note = Synthetic
LCHALVLSAWESCSS

SEQ ID NO: 108 - protein, 15 aa, synthetic construct
  REGION 1..15: note = Synthetic
LCHPLALSAWESCSS

SEQ ID NO: 109 - protein, 15 aa, synthetic construct
  REGION 1..15: note = Synthetic
LCHPLVLSAAESCSS

SEQ ID NO: 110 - protein, 15 aa, synthetic construct
  REGION 1..15: note = Synthetic
LCHPLVLSAWASCSS

SEQ ID NO: 111 - protein, 5 aa, synthetic construct
  REGION 1..5: note = Synthetic
HPLVL

SEQ ID NO: 112 - protein, 15 aa, synthetic construct
  REGION 1..15: note = Synthetic
LEGACLHPLCLWGAG

SEQ ID NO: 113 - protein, 15 aa, synthetic construct
  REGION 1..15: note = Synthetic
LEGWCAHPLCLWGAG

SEQ ID NO: 114 - protein, 15 aa, synthetic construct
  REGION 1..15: note = Synthetic
LEGWCLAPLCLWGAG

SEQ ID NO: 115 - protein, 14 aa, synthetic construct
  REGION 1..14: note = Synthetic
LEGWCLHACLWGAG

SEQ ID NO: 116 - protein, 15 aa, synthetic construct
  REGION 1..15: note = Synthetic
LEGWCLHPACLWGAG

SEQ ID NO: 117 - protein, 15 aa, synthetic construct
  REGION 1..15: note = Synthetic
LEGWCLHPLCAWGAG

SEQ ID NO: 118 - protein, 15 aa, synthetic construct
  REGION 1..15: note = Synthetic
LEGWCLHPLCLAGAG

SEQ ID NO: 119 - protein, 6 aa, synthetic construct
  REGION 1..6: note = Synthetic
CLHPLC

SEQ ID NO: 120 - protein, 270 aa, synthetic construct
  REGION 1..270: note = Synthetic
QGQSGQGLCHPAVLSAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 450 aa, synthetic construct
  REGION 1..450: note = Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 451 aa, synthetic construct
  REGION 1..451: note = Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 15 aa, synthetic construct
  REGION 1..15: note = Synthetic
RASESVDAYGISFMN

SEQ ID NO: 124 - protein, 7 aa, synthetic construct
  REGION 1..7: note = Synthetic
AASNQGS

SEQ ID NO: 125 - protein, 9 aa, synthetic construct
  REGION 1..9: note = Synthetic
QQSKDVPWT

SEQ ID NO: 126 - protein, 10 aa, synthetic construct
  REGION 1..10: note = Synthetic
GFTFSGYAMS

SEQ ID NO: 127 - protein, 10 aa, synthetic construct
  REGION 1..10: note = Synthetic
YISNSGGNAH

SEQ ID NO: 128 - protein, 10 aa, synthetic construct
  REGION 1..10: note = Synthetic
EDYGTSPFVY

SEQ ID NO: 129 - protein, 15 aa, synthetic construct
  REGION 1..15: note = Synthetic
RASESVDSYGISFMN

SEQ ID NO: 130 - protein, 15 aa, synthetic construct
  REGION 1..15: note = Synthetic
AMSGCSWSAFCPYLA

SEQ ID NO: 131 - protein, 15 aa, synthetic construct
  REGION 1..15: note = Synthetic
DVNCAIWYSVCTTVP

SEQ ID NO: 132 - protein, 15 aa, synthetic construct
  REGION 1..15: note = Synthetic
LVCPLYALSSGVCMG

SEQ ID NO: 133 - protein, 15 aa, synthetic construct
  REGION 1..15: note = Synthetic
SVNCRIWSAVCAGYE

SEQ ID NO: 134 - protein, 15 aa, synthetic construct
  REGION 1..15: note = Synthetic
MLVCSLQPTAMCERV

SEQ ID NO: 135 - protein, 15 aa, synthetic construct
  REGION 1..15: note = Synthetic
APRCYMFASYCKSQY

SEQ ID NO: 136 - protein, 15 aa, synthetic construct
  REGION 1..15: note = Synthetic
VGPCELTPKPVCNTY

SEQ ID NO: 137 - protein, 15 aa, synthetic construct
  REGION 1..15: note = Synthetic
ETCNQYERSSGLCFA

SEQ ID NO: 138 - protein, 15 aa, synthetic construct
  REGION 1..15: note = Synthetic
APRTCYTYQCSSFYT

SEQ ID NO: 139 - protein, 15 aa, synthetic construct
  REGION 1..15: note = Synthetic
GLCSWYLSSSGLCVD

SEQ ID NO: 140 - protein, 15 aa, synthetic construct
  REGION 1..15: note = Synthetic
VPWCQLTPRVMCMWA

SEQ ID NO: 141 - protein, 15 aa, synthetic construct
  REGION 1..15: note = Synthetic
NWLDCQFYSECSVYG

SEQ ID NO: 142 - protein, 15 aa, synthetic construct
  REGION 1..15: note = Synthetic
SCPLYVMSSFGGCWD

SEQ ID NO: 143 - protein, 15 aa, synthetic construct
  REGION 1..15: note = Synthetic
MSHCWMFSSSCDGVK

SEQ ID NO: 144 - protein, 15 aa, synthetic construct
  REGION 1..15: note = Synthetic
VSYCTWLIEVTCLRG

SEQ ID NO: 145 - protein, 15 aa, synthetic construct
  REGION 1..15: note = Synthetic
VLCAAYALSSGICGG

SEQ ID NO: 146 - protein, 15 aa, synthetic construct
  REGION 1..15: note = Synthetic
TTCNLYQQSSMFCNA

SEQ ID NO: 147 - protein, 15 aa, synthetic construct
  REGION 1..15: note = Synthetic
APRCYMFASYCKSQY

SEQ ID NO: 148 - protein, 15 aa, synthetic construct
  REGION 1..15: note = Synthetic
PCDQNPYFYPYVCHA

SEQ ID NO: 149 - protein, 15 aa, synthetic construct
  REGION 1..15: note = Synthetic
SVCPMYALSSMLCGA

SEQ ID NO: 150 - protein, 15 aa, synthetic construct
  REGION 1..15: note = Synthetic
LSVECYVFSRCSSLP

SEQ ID NO: 151 - protein, 14 aa, synthetic construct
  REGION 1..14: note = Synthetic
FYCTYLVSLTCHPQ

SEQ ID NO: 152 - protein, 6 aa, synthetic construct
  REGION 1..6: note = Synthetic
GGGSGG

SEQ ID NO: 153 - protein, 6 aa, synthetic construct
  REGION 1..6: note = Synthetic
GSGGGS

SEQ ID NO: 154 - protein, 6 aa, synthetic construct
  REGION 1..6: note = Synthetic
GSGGSG

SEQ ID NO: 155 - protein, 15 aa, synthetic construct
  REGION 1..15: note = Synthetic
SMAGCQWSSFCVQRD

SEQ ID NO: 156 - protein, 15 aa, synthetic construct
  REGION 1..15: note = Synthetic
IYSCYMFASRCTSDK

SEQ ID NO: 157 - protein, 15 aa, synthetic construct
  REGION 1..15: note = Synthetic
SRCSVYEVSSGLCDW

SEQ ID NO: 158 - protein, 15 aa, synthetic construct
  REGION 1..15: note = Synthetic
GMCSAYAYSSKLCTI

SEQ ID NO: 159 - protein, 15 aa, synthetic construct
  REGION 1..15: note = Synthetic
MTTNTCNLLCQQFLT

SEQ ID NO: 160 - protein, 15 aa, synthetic construct
  REGION 1..15: note = Synthetic
FQPCLMFASSCFTSK

SEQ ID NO: 161 - protein, 15 aa, synthetic construct
  REGION 1..15: note = Synthetic
WNCHPAGVGPVFCEV

SEQ ID NO: 162 - protein, 15 aa, synthetic construct
  REGION 1..15: note = Synthetic
ALCSMYLASSGLCNK

SEQ ID NO: 163 - protein, 15 aa, synthetic construct
  REGION 1..15: note = Synthetic
NYLSCQFFQNCYETY

SEQ ID NO: 164 - protein, 15 aa, synthetic construct
  REGION 1..15: note = Synthetic
GWCLFSDMWLGLCSA

SEQ ID NO: 165 - protein, 15 aa, synthetic construct
  REGION 1..15: note = Synthetic
EFCARDWLPYQCSSF

SEQ ID NO: 166 - protein, 15 aa, synthetic construct
  REGION 1..15: note = Synthetic
TSYCSIEHYPCNTHH

SEQ ID NO: 167 - protein, 15 aa, synthetic construct
  REGION 1..15: note = Synthetic
PYICSSFPLDCQAGQ

SEQ ID NO: 168 - protein, 15 aa, synthetic construct
  REGION 1..15: note = Synthetic
VGCEWYMSSSGMCSR

SEQ ID NO: 169 - protein, 15 aa, synthetic construct
  REGION 1..15: note = Synthetic
EVCGGCSMQSVSCWP

SEQ ID NO: 170 - protein, 15 aa, synthetic construct
  REGION 1..15: note = Synthetic
FTECQLSPKAICMSN

SEQ ID NO: 171 - protein, 15 aa, synthetic construct
  REGION 1..15: note = Synthetic
KYCLFSEYVEGTCLN

SEQ ID NO: 172 - protein, 15 aa, synthetic construct
  REGION 1..15: note = Synthetic
SGCPMYAWGWDECWR

SEQ ID NO: 173 - protein, 15 aa, synthetic construct
  REGION 1..15: note = Synthetic
VDCPWYASSSAICSR

SEQ ID NO: 174 - protein, 15 aa, synthetic construct
  REGION 1..15: note = Synthetic
DMLLCQIRGSCAAWG

SEQ ID NO: 175 - protein, 15 aa, synthetic construct
  REGION 1..15: note = Synthetic
ECHPYQASASLWCGY

SEQ ID NO: 176 - protein, 15 aa, synthetic construct
  REGION 1..15: note = Synthetic
MMMGCMWSAWCPPSR

SEQ ID NO: 177 - protein, 15 aa, synthetic construct
  REGION 1..15: note = Synthetic
NAYFRCSLMCNMIMF

SEQ ID NO: 178 - protein, 15 aa, synthetic construct
  REGION 1..15: note = Synthetic
ACCKESVHSVHDCKR

SEQ ID NO: 179 - protein, 15 aa, synthetic construct
  REGION 1..15: note = Synthetic
ACIGINSYMSNYCYL

SEQ ID NO: 180 - protein, 15 aa, synthetic construct
  REGION 1..15: note = Synthetic
ANCSFLELTNKFCTI

SEQ ID NO: 181 - protein, 15 aa, synthetic construct
  REGION 1..15: note = Synthetic
AYCSYLMFASNPCII

SEQ ID NO: 182 - protein, 15 aa, synthetic construct
  REGION 1..15: note = Synthetic
CFTSKCPCLCYSLLA

SEQ ID NO: 183 - protein, 15 aa, synthetic construct
  REGION 1..15: note = Synthetic
CLCRDINCWLGCSKT

SEQ ID NO: 184 - protein, 15 aa, synthetic construct
  REGION 1..15: note = Synthetic
CWCDIYCSPYQCSSF

SEQ ID NO: 185 - protein, 15 aa, synthetic construct
  REGION 1..15: note = Synthetic
DCIYYYQQSANLCSY

SEQ ID NO: 186 - protein, 15 aa, synthetic construct
  REGION 1..15: note = Synthetic
DCTGVNYYIDKHCTN

SEQ ID NO: 187 - protein, 15 aa, synthetic construct
  REGION 1..15: note = Synthetic
DECHGYLRSSGLCGG

SEQ ID NO: 188 - protein, 15 aa, synthetic construct
  REGION 1..15: note = Synthetic
DICSAYAASSGFCYY

SEQ ID NO: 189 - protein, 15 aa, synthetic construct
  REGION 1..15: note = Synthetic
DIICVLTPTAWCGRT

SEQ ID NO: 190 - protein, 15 aa, synthetic construct
  REGION 1..15: note = Synthetic
DNCCMYCSWWIACRD

SEQ ID NO: 191 - protein, 15 aa, synthetic construct
  REGION 1..15: note = Synthetic
DSCQWYMLSADLCGT

SEQ ID NO: 192 - protein, 15 aa, synthetic construct
  REGION 1..15: note = Synthetic
DSVCFSSSSFLCHKS

SEQ ID NO: 193 - protein, 15 aa, synthetic construct
  REGION 1..15: note = Synthetic
DTMCAIWWTVCSGGR

SEQ ID NO: 194 - protein, 15 aa, synthetic construct
  REGION 1..15: note = Synthetic
ECTYQTSSFHEACMS

SEQ ID NO: 195 - protein, 15 aa, synthetic construct
  REGION 1..15: note = Synthetic
EGCNLYERSSYGCNN

SEQ ID NO: 196 - protein, 15 aa, synthetic construct
  REGION 1..15: note = Synthetic
EGCTAFAMSAGICGG

SEQ ID NO: 197 - protein, 15 aa, synthetic construct
  REGION 1..15: note = Synthetic
EQSCSLTPIAFCWSE

SEQ ID NO: 198 - protein, 15 aa, synthetic construct
  REGION 1..15: note = Synthetic
EWCNAYISSSKLCST

SEQ ID NO: 199 - protein, 15 aa, synthetic construct
  REGION 1..15: note = Synthetic
FEVCYMFASACRNGM

SEQ ID NO: 200 - protein, 15 aa, synthetic construct
  REGION 1..15: note = Synthetic
FSCSWYAESSSLCDI

SEQ ID NO: 201 - protein, 15 aa, synthetic construct
  REGION 1..15: note = Synthetic
FVCQMFEASSGLCGG

SEQ ID NO: 202 - protein, 15 aa, synthetic construct
  REGION 1..15: note = Synthetic
FYCPCCMFASSCGSR

SEQ ID NO: 203 - protein, 15 aa, synthetic construct
  REGION 1..15: note = Synthetic
FYCSYLPGASHQCSH

SEQ ID NO: 204 - protein, 15 aa, synthetic construct
  REGION 1..15: note = Synthetic
FYCSYLYMCEVCCYE

SEQ ID NO: 205 - protein, 15 aa, synthetic construct
  REGION 1..15: note = Synthetic
GFCTQHTVLTWCPTS

SEQ ID NO: 206 - protein, 15 aa, synthetic construct
  REGION 1..15: note = Synthetic
GSCPSYAVSAGLCYA

SEQ ID NO: 207 - protein, 15 aa, synthetic construct
  REGION 1..15: note = Synthetic
GSQCFLTPTAFCTHT

SEQ ID NO: 208 - protein, 15 aa, synthetic construct
  REGION 1..15: note = Synthetic
GTCHPYMQSSKICNN

SEQ ID NO: 209 - protein, 15 aa, synthetic construct
  REGION 1..15: note = Synthetic
GVECFVFTGGCGGYG

SEQ ID NO: 210 - protein, 15 aa, synthetic construct
  REGION 1..15: note = Synthetic
HELCNGHWVPCCWAY

SEQ ID NO: 211 - protein, 15 aa, synthetic construct
  REGION 1..15: note = Synthetic
ICDSYYAVSSGLCLL

SEQ ID NO: 212 - protein, 15 aa, synthetic construct
  REGION 1..15: note = Synthetic
IGCAWYVSSAGWCSP

SEQ ID NO: 213 - protein, 15 aa, synthetic construct
  REGION 1..15: note = Synthetic
INLCWMFASECGEHH

SEQ ID NO: 214 - protein, 15 aa, synthetic construct
  REGION 1..15: note = Synthetic
KCWLAEMTNLEHCNM

SEQ ID NO: 215 - protein, 15 aa, synthetic construct
  REGION 1..15: note = Synthetic
KHCSDFAYSSRLCDR

SEQ ID NO: 216 - protein, 15 aa, synthetic construct
  REGION 1..15: note = Synthetic
KVCSSYASSSGLCGW

SEQ ID NO: 217 - protein, 15 aa, synthetic construct
  REGION 1..15: note = Synthetic
LDSCYMFASYCVQAV

SEQ ID NO: 218 - protein, 15 aa, synthetic construct
  REGION 1..15: note = Synthetic
LLACHPIFVTVCQTR

SEQ ID NO: 219 - protein, 15 aa, synthetic construct
  REGION 1..15: note = Synthetic
LLSCPYNPEHVCHTS

SEQ ID NO: 220 - protein, 15 aa, synthetic construct
  REGION 1..15: note = Synthetic
LMCSLYALSSNLCGR

SEQ ID NO: 221 - protein, 15 aa, synthetic construct
  REGION 1..15: note = Synthetic
LMWCVLFLWSWCCRI

SEQ ID NO: 222 - protein, 15 aa, synthetic construct
  REGION 1..15: note = Synthetic
LPICHLTPTAVCTHI

SEQ ID NO: 223 - protein, 15 aa, synthetic construct
  REGION 1..15: note = Synthetic
LSNMCLAFGSCLYAW

SEQ ID NO: 224 - protein, 15 aa, synthetic construct
  REGION 1..15: note = Synthetic
LSRCHPIWYTICQNP

SEQ ID NO: 225 - protein, 15 aa, synthetic construct
  REGION 1..15: note = Synthetic
LTQCMSVHKECGGYE

SEQ ID NO: 226 - protein, 15 aa, synthetic construct
  REGION 1..15: note = Synthetic
LVNCRIWSWVCEEAT

SEQ ID NO: 227 - protein, 15 aa, synthetic construct
  REGION 1..15: note = Synthetic
LYCSWYQMSSAVCKE

SEQ ID NO: 228 - protein, 15 aa, synthetic construct
  REGION 1..15: note = Synthetic
MECGWYALSARFCEV

SEQ ID NO: 229 - protein, 15 aa, synthetic construct
  REGION 1..15: note = Synthetic
MTCSPYAMSAHFCNE

SEQ ID NO: 230 - protein, 15 aa, synthetic construct
  REGION 1..15: note = Synthetic
MVCSLYAYSASLCGA

SEQ ID NO: 231 - protein, 15 aa, synthetic construct
  REGION 1..15: note = Synthetic
NALCWSTFSWWCDMD

SEQ ID NO: 232 - protein, 15 aa, synthetic construct
  REGION 1..15: note = Synthetic
NFTCMLTPKAYCVQT

SEQ ID NO: 233 - protein, 15 aa, synthetic construct
  REGION 1..15: note = Synthetic
NGACIFTLSWCTNKT

SEQ ID NO: 234 - protein, 15 aa, synthetic construct
  REGION 1..15: note = Synthetic
NGCELYAAASGLCRT

SEQ ID NO: 235 - protein, 15 aa, synthetic construct
  REGION 1..15: note = Synthetic
NIECSVFGRCCCDNY

SEQ ID NO: 236 - protein, 15 aa, synthetic construct
  REGION 1..15: note = Synthetic
PACRPMFWNRSCDNI

SEQ ID NO: 237 - protein, 15 aa, synthetic construct
  REGION 1..15: note = Synthetic
PCRVSNMFFPYNCLD

SEQ ID NO: 238 - protein, 15 aa, synthetic construct
  REGION 1..15: note = Synthetic
PIMCMLLPESYCWIW

SEQ ID NO: 239 - protein, 15 aa, synthetic construct
  REGION 1..15: note = Synthetic
PQSCYMFASLCMPNG

SEQ ID NO: 240 - protein, 15 aa, synthetic construct
  REGION 1..15: note = Synthetic
PRCPQGLPLYQCSSF

SEQ ID NO: 241 - protein, 15 aa, synthetic construct
  REGION 1..15: note = Synthetic
PSVECLVFKRCYALP

SEQ ID NO: 242 - protein, 15 aa, synthetic construct
  REGION 1..15: note = Synthetic
PVCQRSATIYNCNWF

SEQ ID NO: 243 - protein, 15 aa, synthetic construct
  REGION 1..15: note = Synthetic
QCAAYYISSFGGCSN

SEQ ID NO: 244 - protein, 15 aa, synthetic construct
  REGION 1..15: note = Synthetic
QFGCFMLARDFCGTY

SEQ ID NO: 245 - protein, 15 aa, synthetic construct
  REGION 1..15: note = Synthetic
QMMCPYNPEHKCHQK

SEQ ID NO: 246 - protein, 15 aa, synthetic construct
  REGION 1..15: note = Synthetic
QRECWMFASSCNSKN

SEQ ID NO: 247 - protein, 15 aa, synthetic construct
  REGION 1..15: note = Synthetic
QSNMCTTYICSSFNY

SEQ ID NO: 248 - protein, 15 aa, synthetic construct
  REGION 1..15: note = Synthetic
QSRCHSLAPYLCSSF

SEQ ID NO: 249 - protein, 15 aa, synthetic construct
  REGION 1..15: note = Synthetic
RAYCSLLFADSCNNN

SEQ ID NO: 250 - protein, 15 aa, synthetic construct
  REGION 1..15: note = Synthetic
RCIGINQYIDSNCYN

SEQ ID NO: 251 - protein, 15 aa, synthetic construct
  REGION 1..15: note = Synthetic
RLSCFMFASQCALEF

SEQ ID NO: 252 - protein, 15 aa, synthetic construct
  REGION 1..15: note = Synthetic
RQCIILMNHRQCFFK

SEQ ID NO: 253 - protein, 15 aa, synthetic construct
  REGION 1..15: note = Synthetic
RSCTPYMMSSSLCNT

SEQ ID NO: 254 - protein, 15 aa, synthetic construct
  REGION 1..15: note = Synthetic
RYCHYWKMPYECSSF

SEQ ID NO: 255 - protein, 15 aa, synthetic construct
  REGION 1..15: note = Synthetic
SCVSLSWFDMLKCYE

SEQ ID NO: 256 - protein, 15 aa, synthetic construct
  REGION 1..15: note = Synthetic
SDNCEIWWTVCSAAM

SEQ ID NO: 257 - protein, 15 aa, synthetic construct
  REGION 1..15: note = Synthetic
SFCWSYLVSSGLCGV

SEQ ID NO: 258 - protein, 15 aa, synthetic construct
  REGION 1..15: note = Synthetic
SMCMNNYGTTIMCGN

SEQ ID NO: 259 - protein, 15 aa, synthetic construct
  REGION 1..15: note = Synthetic
SMVGCGWSTFCPSRG

SEQ ID NO: 260 - protein, 15 aa, synthetic construct
  REGION 1..15: note = Synthetic
SSLHCANGHTCPFCL

SEQ ID NO: 261 - protein, 15 aa, synthetic construct
  REGION 1..15: note = Synthetic
SVCSYYEESSGICSP

SEQ ID NO: 262 - protein, 15 aa, synthetic construct
  REGION 1..15: note = Synthetic
SWCGWYAASSGVCAL

SEQ ID NO: 263 - protein, 15 aa, synthetic construct
  REGION 1..15: note = Synthetic
TCISQTIDSYLNCVN

SEQ ID NO: 264 - protein, 15 aa, synthetic construct
  REGION 1..15: note = Synthetic
TFCNLYTKSSNICMS

SEQ ID NO: 265 - protein, 15 aa, synthetic construct
  REGION 1..15: note = Synthetic
TYCVFHEYLDNTCNN

SEQ ID NO: 266 - protein, 15 aa, synthetic construct
  REGION 1..15: note = Synthetic
VATGCPNLMLCGSWP

SEQ ID NO: 267 - protein, 15 aa, synthetic construct
  REGION 1..15: note = Synthetic
VEYCSLLLGNRCDYW

SEQ ID NO: 268 - protein, 15 aa, synthetic construct
  REGION 1..15: note = Synthetic
VGCNMYLMSAGLCVD

SEQ ID NO: 269 - protein, 15 aa, synthetic construct
  REGION 1..15: note = Synthetic
VLYCSWDSGTCVGSH

SEQ ID NO: 270 - protein, 15 aa, synthetic construct
  REGION 1..15: note = Synthetic
VMFSCYYLETCAPGV

SEQ ID NO: 271 - protein, 15 aa, synthetic construct
  REGION 1..15: note = Synthetic
VRIGLCPESCLVSGF

SEQ ID NO: 272 - protein, 15 aa, synthetic construct
  REGION 1..15: note = Synthetic
VTCTYYATSSSLCNT

SEQ ID NO: 273 - protein, 15 aa, synthetic construct
  REGION 1..15: note = Synthetic
VTGCILLPKAWCWGD

SEQ ID NO: 274 - protein, 15 aa, synthetic construct
  REGION 1..15: note = Synthetic
VWCSIYEYSSNLCSR

SEQ ID NO: 275 - protein, 15 aa, synthetic construct
  REGION 1..15: note = Synthetic
WMLECQYNNTCNNMT

SEQ ID NO: 276 - protein, 15 aa, synthetic construct
  REGION 1..15: note = Synthetic
WPCSPLEYYNNICNV

SEQ ID NO: 277 - protein, 15 aa, synthetic construct
  REGION 1..15: note = Synthetic
WTYDCHLNQTCPTYY

SEQ ID NO: 278 - protein, 15 aa, synthetic construct
  REGION 1..15: note = Synthetic
YCSINMYLIGGNCMY

SEQ ID NO: 279 - protein, 15 aa, synthetic construct
  REGION 1..15: note = Synthetic
YFCSLYANSAGFCGG

SEQ ID NO: 280 - protein, 15 aa, synthetic construct
  REGION 1..15: note = Synthetic
YVSCYMFSSSCPSTW

SEQ ID NO: 281 - protein, 265 aa, synthetic construct
  REGION 1..265: note = Synthetic
QGQSGQGTSYCSIEHYPCNTHHGGGSSGGSISSGLLSGRSDNPGGGSDIQLTQSPSSLSASVGDRVTITCRASESVDAYGISFMNWFQQKPGKAPKLLIYAASNQGSGVPSRFSGSGSGTDFTLTISSMQPEDFATYYCQQSKDVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 445 aa, synthetic construct
  REGION 1..445: note = Synthetic
EVQLVESGGGLVQPGGSLRLSCAASGFTFSGYAMSWVRQAPGKGLEWVAYISNSGGNAHYADSVKGRFTISRDNSKNTLYLQMNSLRAEDTAVYYCTREDYGTSPFVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 283 - protein, 11 aa, synthetic construct
  REGION 1..11: note = Synthetic
RASQSISSYLN

SEQ ID NO: 284 - protein, 7 aa, synthetic construct
  REGION 1..7: note = Synthetic
AASSLQS

SEQ ID NO: 285 - protein, 7 aa, synthetic construct
  REGION 1..7: note = Synthetic
DNGYPST

SEQ ID NO: 286 - protein, 5 aa, synthetic construct
  REGION 1..5: note = Synthetic
SYAMS

SEQ ID NO: 287 - protein, 15 aa, synthetic construct
  REGION 1..15: note = Synthetic
SSIWRNGIVTVYADS

SEQ ID NO: 288 - protein, 7 aa, synthetic construct
  REGION 1..7: note = Synthetic
WSAAFDY

SEQ ID NO: 289 - protein, 15 aa, synthetic construct
  REGION 1..15: note = Synthetic
YCEVSELFVLPWCMG

SEQ ID NO: 290 - protein, 15 aa, synthetic construct
  REGION 1..15: note = Synthetic
SCLMHPHYAHDYCYV

SEQ ID NO: 291 - protein, 15 aa, synthetic construct
  REGION 1..15: note = Synthetic
LCEVLMLLQHPWCMG

SEQ ID NO: 292 - protein, 15 aa, synthetic construct
  REGION 1..15: note = Synthetic
IACRHFMEQLPFCHH

SEQ ID NO: 293 - protein, 15 aa, synthetic construct
  REGION 1..15: note = Synthetic
FGPRCGEASTCVPYE

SEQ ID NO: 294 - protein, 15 aa, synthetic construct
  REGION 1..15: note = Synthetic
ILYCDSWGAGCLTRP

SEQ ID NO: 295 - protein, 15 aa, synthetic construct
  REGION 1..15: note = Synthetic
GIALCPSHFCQLPQT

SEQ ID NO: 296 - protein, 15 aa, synthetic construct
  REGION 1..15: note = Synthetic
DGPRCFVSGECSPIG

SEQ ID NO: 297 - protein, 15 aa, synthetic construct
  REGION 1..15: note = Synthetic
LCYKLDYDDRSYCHI

SEQ ID NO: 298 - protein, 15 aa, synthetic construct
  REGION 1..15: note = Synthetic
PCHPHPYDARPYCNV

SEQ ID NO: 299 - protein, 15 aa, synthetic construct
  REGION 1..15: note = Synthetic
PCYWHPFFAYRYCNT

SEQ ID NO: 300 - protein, 15 aa, synthetic construct
  REGION 1..15: note = Synthetic
VCYYMDWLGRNWCSS

SEQ ID NO: 301 - protein, 15 aa, synthetic construct
  REGION 1..15: note = Synthetic
LCDLFKLREFPYCMG

SEQ ID NO: 302 - protein, 15 aa, synthetic construct
  REGION 1..15: note = Synthetic
YLPCHFVPIGACNNK

SEQ ID NO: 303 - protein, 15 aa, synthetic construct
  REGION 1..15: note = Synthetic
IFCHMGVVVPQCANY

SEQ ID NO: 304 - protein, 15 aa, synthetic construct
  REGION 1..15: note = Synthetic
ACHPHPYDARPYCNV

SEQ ID NO: 305 - protein, 15 aa, synthetic construct
  REGION 1..15: note = Synthetic
PCHPAPYDARPYCNV

SEQ ID NO: 306 - protein, 15 aa, synthetic construct
  REGION 1..15: note = Synthetic
PCHPHAYDARPYCNV

SEQ ID NO: 307 - protein, 15 aa, synthetic construct
  REGION 1..15: note = Synthetic
PCHPHPADARPYCNV

SEQ ID NO: 308 - protein, 15 aa, synthetic construct
  REGION 1..15: note = Synthetic
PCHPHPYAARPYCNV

SEQ ID NO: 309 - protein, 15 aa, synthetic construct
  REGION 1..15: note = Synthetic
PCHPHPYDAAPYCNV

SEQ ID NO: 310 - protein, 15 aa, synthetic construct
  REGION 1..15: note = Synthetic
PCHPHPYDARPACNV

SEQ ID NO: 311 - protein, 15 aa, synthetic construct
  REGION 1..15: note = Synthetic
PCHPHPYDARPYCAV

SEQ ID NO: 312 - protein, 15 aa, synthetic construct
  REGION 1..15: note = Synthetic
PCHAHPYDARPYCNV

SEQ ID NO: 313 - protein, 15 aa, synthetic construct
  REGION 1..15: note = Synthetic
PCHPHPYDARAYCNV

SEQ ID NO: 314 - protein, 264 aa, synthetic construct
  REGION 1..264: note = Synthetic
QGQSGSGIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 315 - protein, 442 aa, synthetic construct
  REGION 1..442: note = Synthetic
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG